(19) United States
(12) Patent Application Publication
(10) Pub. No.: US 2020/0260698 A1
(43) Pub. Date: Aug. 20, 2020

(54) EXON DELETION CORRECTION OF DUCHENNE MUSCULAR DYSTROPHY MUTATIONS IN THE DYSTROPHIN ACTIN BINDING DOMAIN 1 USING CRISPR GENOME EDITING

(71) Applicant: The Board of Regents of the University of Texas System, Austin, TX (US)

(72) Inventors: Viktoriia KYRYCHENKO, Dallas, TX (US); Eric N. OLSON, University Park, TX (US); Rhonda BASSEL-DUBY, Dallas, TX (US)

(73) Assignee: The Board of Regents of the University of Texas System, Austin, TX (US)

(21) Appl. No.: 16/639,828

(22) PCT Filed: Aug. 17, 2018
(86) PCT No.: PCT/US2018/046884

§ 371 Date: Feb. 18, 2020

Related U.S. Application Data
(60) Provisional application No. 62/547,590, filed on Aug. 18, 2017.

(51) Int. Cl. A01K 67/027; C12N 15/10; C12N 15/113; A61P 21/00

(52) U.S. Cl. CPC A01K 67/0276; C12N 15/102; C12N 15/113; A61P 21/00; A01K 2217/075; C12N 2330/51; A01K 2267/0306; C12N 2310/20; C12N 2320/32; C12N 2320/33; A01K 2227/105

(57) ABSTRACT

CRISPR/Cas9-mediated genome editing holds clinical potential for treating genetic diseases, such as Duchenne muscular dystrophy (DMD), which is caused by mutations in the dystrophin gene and absence or deficiency of dystrophin protein in striated muscle. Provided herein are compositions and methods for treating DMD caused by mutations in the dystrophin Actin Binding Domain 1 (ABD-1). The compositions and method described herein can be used to remove mutant sequences in dystrophin ABD-1 to generate a corrected DMD protein that, while lacking one or more exons (e.g., exons 3-9), retains important functional properties.

### PRIORITY CLAIM

The present application claims benefit of priority to U.S. Provisional Application Ser. No. 62/547,590, filed on Aug. 18, 2017, the entire contents of which are hereby incorporated by reference in their entireties.

### FEDERAL FUNDING SUPPORT CLAUSE

This invention was made with government support under grant no. U54 HD 087351 awarded by National Institutes of Health. The government has certain rights in the invention.

### SEQUENCE LISTING

The instant application contains a sequence listing which has been submitted electronically in ASCII format and is hereby incorporated by reference in its entirety. Said ASCII copy, created on Aug. 16, 2018, is named EXON_008_01WO_SeqList_ST25.txt and is 1 MB in size.

### FIELD OF THE DISCLOSURE

The present disclosure relates to the fields of molecular biology, medicine and genetics. More particularly, the disclosure relates to the use of genome editing to correct mutations in actin binding domain 1 of the DMD gene using an exon-deletion approach.

### BACKGROUND

Dystrophin is a large intracellular protein that stabilizes muscle membranes against forces associated with contraction and stretch by providing a mechanical link between the intracellular actin cytoskeleton and the transmembrane dystroglycan complex. The DMD gene, one of the largest human genes, encodes dystrophin and is comprised of 79 exons on the Xp21 chromosome. Mutations that disrupt the open reading frame of dystrophin lead to Duchenne muscular dystrophy (DMD), a life-limiting, rapidly progressive form of muscular dystrophy. DMD is a X-linked recessive disorder affecting 1:5,000 boys. Loss of dystrophin destabilizes muscle membranes, allowing excess calcium into cardiac and skeletal muscle cells, resulting in muscle degeneration and necrosis. Internal deletion mutations in the dystrophin gene (DMD) that preserve the amino- and carboxyl-termini of the protein but eliminate various internal rod domains cause Becker muscular dystrophy (BMD), a relatively mild muscle disease.

There remains a need in the art for compositions and methods for treating DMD.

### SUMMARY

Thus, in accordance with the present disclosure, there is provided a composition comprising a sequence encoding a first DMD guide RNA (gRNA) targeting a first genomic target sequence; a sequence encoding a second DMD gRNA targeting a second genomic target sequence; a sequence encoding a first promoter, wherein the first promoter drives expression of the sequence encoding the first DMD gRNA; and a sequence encoding a second promoter, wherein the second promoter drives expression of the sequence encoding the second DMD gRNA; wherein the first genomic target sequence is in any one of introns 2-9 of the dystrophin gene; wherein the second genomic target sequence is in any one of introns 2-9 of the dystrophin gene. In some embodiments, the first genomic target sequence is in intron 2 and the second genomic target sequence is in intron 7 of the dystrophin gene. In some embodiments, the first genomic target sequence is in intron 5 and the second genomic target sequence is in intron 7 of the dystrophin gene. In some embodiments, the first genomic target sequence is in intron 2 and the second genomic target sequence is in intron 9 of the dystrophin gene. In some embodiments, the first genomic target sequence is in intron 7 and the second genomic target sequence is in intron 9 of the dystrophin gene. In some embodiments, the first genomic target sequence is located 5′ from a wildtype exon, and the second genomic target sequence is located 3′ from the wildtype exon. The wildtype exon may be exon 2, 3, 4, 5, 6, 7, 8 or 9 of dystrophin. In some embodiments, the first genomic target sequence is located 5′ from an exon comprising a mutation, and the second genomic target sequence is located 3′ from the exon comprising a mutation. The exon comprising a mutation may be exon 2, 3, 4, 5, 6, 7, 8 or 9 of dystrophin. In some embodiments, the sequence encoding the first DMD gRNA and the sequence encoding the second DMD gRNA are identical. In some embodiments, the sequence encoding the first DMD gRNA and the sequence encoding the second DMD gRNA are not identical. In some embodiments, the sequence encoding the first DMD guide RNA is any one of SEQ ID NO: 1 to 5. In some embodiments, the sequence encoding the second DMD guide RNA is any one of SEQ ID NO: 1 to 5. In some embodiments, the sequence of the first DMD guide RNA is any one of SEQ ID NO: 6 to 10. In some embodiments, the sequence of the second DMD guide RNA is any one of SEQ ID NO: 6 to 10. In some embodiments, the sequence encoding the first promoter and the sequence encoding the second promoter are identical. In some embodiments, the sequence encoding the first promoter and the sequence encoding the second promoter are not identical. In some embodiments, at least one of the first promoter and the second promoter is a constitutive promoter. In some embodiments, at least one of the first promoter and the second promoter is an inducible promoter. In some embodiments, at least one of the first promoter and the second promoter is a muscle-specific promoter. In some embodiments, at least one of the first promoter and the second promoter is CK8. In some embodiments, at least one of the first promoter and the second promoter is CK8e. In some embodiments, at least one of the first promoter and the second promoter is selected from the group consisting of the U6 promoter, the H1 promoter, and the 7SK promoter. In some embodiments, a first vector comprises the sequence encoding the first DMD gRNA and the first promoter, and a second vector comprises the sequence encoding the second DMD gRNA and the second promoter. In some embodiments, at least one of the first vector and the second vector is a non-viral vector. In some embodiments, the non-viral vector is a plasmid. In some embodiments, a liposome or nanoparticle comprises the non-viral vector. In some embodiments, at least one of the first vector and the second vector is a viral vector. In some embodiments, the viral vector is an adeno-associated viral (AAV) vector. In some embodiments, the AAV vector is replication-defective or conditionally replication defective. In some embodiments, the AAV vector is a recombinant AAV vector. In some embodiments, the AAV vector comprises a sequence isolated or derived from an AAV vector of serotype AAV1, AAV2, AAV3, AAV4, AAV5, AAV6, AAV7, AAV8, AAV9, AAV10, AAV11, AAVRh.74 or any combination thereof. In some embodiments, a first vector comprises the sequence encoding the first DMD gRNA, the sequence encoding the second DMD gRNA, the sequence encoding the first promoter, and the sequence encoding the second promoter. In some embodiments, the composition further comprises a sequence encoding a nuclease, such as Cas9. In some embodiments, the sequence encoding the Cas9 is isolated or derived from S. aureus. In some embodiments, the composition comprises a pharmaceutically-acceptable carrier.

Also provided is a cell comprising a composition of the disclosure. The cell may be a mammalian cell, such as a murine cell or a human cell. In some embodiments, the cell is an oocyte (e.g., a non-human oocyte). Also provided are compositions comprising a cell of the disclosure, and a genetically engineered mouse comprising a cell of the disclosure.

Also provided is a method of treating a subject in need thereof, the method comprising administering to the subject a therapeutically effective amount of a composition of the disclosure. In some embodiments, the composition is administered systemically. In some embodiments, the composition is administered by an intravenous infusion or injection. In some embodiments, the composition is administered locally. In some embodiments, the composition is administered directly to a muscle tissue, such as a tibialis anterior tissue, a quadriceps tissue, a soleus tissue, a diaphragm tissue, or a heart tissue. In some embodiments, the composition is administered by an intramuscular infusion or injection. In some embodiments, the composition is administered by intra-cardiac injection. In some embodiments, the subject is a neonate, an infant, a child, a young adult, or an adult. In some embodiments, the subject has muscular dystrophy. In some embodiments, the subject is a genetic carrier for muscular dystrophy. In some embodiments, the subject is male. In some embodiments, the subject is female. In some embodiments, the subject is less than 10 years old, less than 5 years old, or less than 2 years old.

Also provided herein is the use of the composition of the disclosure in the manufacture of a medicament for the treatment of muscular dystrophy.

Also provided herein is a genetically engineered mouse whose genome comprises a deletion of exon 8 and 9 of the dystrophin gene resulting in an out of frame shift and a premature stop codon in exon 10.

Also provided herein is a genetically engineered mouse produced by a method comprising the steps of: (a) contacting a fertilized oocyte with (i) a Cas9, a first gRNA, and a second gRNA, or (ii) one or more sequences encoding the same, thereby creating a modified oocyte, wherein the first gRNA targets an intron located 5′ to exon 8 of the dystrophin gene, wherein the second gRNA targets an intron located 3′ to exon 9 of the dystrophin gene, wherein the contacting causes exons 8 and 9 to be deleted in the modified oocyte, wherein deletion of exons 8 and 9 results in an out of frame shift and a premature stop codon in exon 10; and (b) transferring the modified oocyte into a recipient female. The disclosure also provides a mouse produced by this method.

In some embodiments, the disclosure provides a method of editing an Actin Binding Domain 1 (ABD-1) dystrophin gene defect in a subject comprising contacting a cell with one or more expression constructs expressing Cas9, a first guide RNA and a second guide RNA, wherein the first guide RNA targets a dystrophin intron 5′ to the gene defect, and the second guide RNA targets a dystrophin intron 3′ to the gene defect, thereby resulting in an edited dystrophin gene lacking dystrophin exons 3-9. The cell may be a muscle cell, a satellite cell, or an iPSC/iPSC-derived CM. In some embodiments, the Cas9 expression construct is distinct from the expression construct that expresses the first and/or second guide RNAs. In some embodiments, the Cas9 expression construct is the same expression construct as that expressing the first and/or second guide RNAs. In some embodiments, the expression construct(s) is/are a viral vector. In some embodiments, the expression construct(s) is/are a non-viral vector. In some embodiments, the expression construct(s) is/are naked plasmid DNA or chemically-modified mRNA. In some embodiments, (a) the first guide RNA targets dystrophin intron 2 and the second guide RNA targets dystrophin intron 9; (b) the first guide RNA targets dystrophin intron 2, and the second guide RNA targets dystrophin intron 7; (c) the first guide RNA targets dystrophin intron 7, and the second guide RNA targets dystrophin intron 9. In some embodiments, the expression construct(s) is/are provided to the cell in one or more nanoparticles. In some embodiments, the viral vector is an AAV vector, such as AAV-9. In some embodiments, the contacting comprises administration of AAV vector to the subject, such as by intra-muscular, intra-peritoneal (IP), retro-orbital (RO), or intra-cardiac injection. In some embodiments, the expression construct(s) is/are delivered to and iPSC/iPSC-derived CM or directly to a muscle tissue, such as tibialis anterior, quadriceps, soleus, diaphragm or heart. In some embodiments, the expression construct(s) is/are delivered systemically. In some embodiments, the subject exhibits normal dystrophin-positive myofibers and/or mosaic dystrophin-positive myofibers containing centralized nuclei. In some embodiments, the subject exhibits a decreased serum CK level as compared to a serum CK level prior to contacting. In some embodiments, the subject exhibits improved grip strength as compared to a serum CK level prior to contacting. In some embodiments, the first guide RNA is encoded by the DNA sequence 5′-AATTAATCTGCCGAAGATGA-3′ (SEQ ID NO: 1). In some embodiments, the second guide RNA is encoded by the DNA sequence 5′-AAACAAACCAGCTCTTCACG-3′ (SEQ ID NO: 5). In some embodiments, the expression construct(s) is/are delivered to a human iPS cell by nucleofection. In some embodiments, the method comprises further comprising identifying an ABD-1 target based on reference to a Duchenne mutation database. In some embodiments, the first and second guide RNAs are encoded by and expressed from the same expression construct, or from distinct expression constructs. In some embodiments, one or more promoters in the expression construct(s) is/are RNA polymerase III promoters. In some embodiments, the mutant dystrophin exon is exon 3, 4, 5, 6, 7, 8 or 9.

Other objects, features and advantages of the present disclosure will become apparent from the following detailed description. It should be understood, however, that the detailed description and the specific examples, while indicating preferred embodiments, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1A-F. Generating an iDMD model by deleting DMD exons 8-9 using CRISPR/Cas9-mediated genome editing. (FIG. 1A) Strategy showing CRISPR/Cas9-mediated genomic editing of wild type (WT) DMD to generate ΔEx8-9 iDMD. Shape and color of boxes denoting DMD exons indicate reading frame and protein coding domains. Yellow designates actin binding domain-1 (ABD-1). Blue marks part of the central rod domain. Red lines indicate actin binding sites (ABS1, ABS2 and ABS3). Arrowheads mark targeting site of guide RNAs (gRNAs). Stop sign marks exon with stop codon. (FIG. 1B) Sequences of gRNAs (SEQ ID NO: 93, SEQ ID NO: 5) and their targeting sites within intron 7 (top, SEQ ID NO: 94-45) and intron 9 (bottom, SEQ ID NO: 96-97). gRNAs were designed to target 3′ region of intron 7 (gRNA-7) and 5′ region of intron 9 (gRNA-9). PAM sites are highlighted in red. (FIG. 1C) PCR genotyping of control and ΔEx8-9 iDMD induced pluripotent stem cell (iPSC) lines using primers upstream and downstream of the gRNA targeting sites (top) and within intron 7 flanking the gRNA-7 targeting site (bottom). Sequencing of PCR product of ΔEx8-9 iDMD validates splicing of intron 7 to intron 9 (SEQ ID NO: 98). PCR primers are indicated by arrows. Arrowhead indicates gRNA targeting site. M denotes marker lane. (FIG. 1D) RT-PCR analysis of dystrophin mRNA expression in control and ΔEx8-9 iDMD iPSC-derived cardiomyocytes. Forward primer targeting exon 1 and reverse primer targeting exon 10 were used. Sequencing confirmed splicing of exon 7 to exon 10, introducing a stop-codon (SEQ ID NO: 99-100). (FIG. 1E) Western blot analysis showing dystrophin protein expression in iPSC-derived cardiomyocytes using anti-dystrophin antibody. Vinculin was used as loading control. n=7 for each group. (FIG. 1F) Immunocytochemistry representations of iPSC-derived cardiomyocytes with anti-dystrophin (red) and anti-troponin I (green). Nuclei are stained with Hoechst 33342 (blue). Scale bar=50 microns. n=4 for each group.

FIG. 2A-F. Correcting ΔEx8-9 iDMD by exon deletion of exons 3-9 to restore dystrophin expression. (FIG. 2A) Three strategies were used to correct ΔEx8-9 iDMD by CRISPR/Cas9-mediated genomic editing: (1) deleting exons 3-7 to generate ΔEx3-9, (2) deleting exons 6-7 to generate ΔEx6-9, and (3) deleting exons 7-11 to generate ΔEx7-11. Shape and color of boxes denoting DMD exons indicate reading frame and protein coding domains. Yellow designates actin binding domain-1 (ABD-1). Blue marks part of the central rod domain. Red lines indicate actin binding sites (ABS1, ABS2 and ABS3). Red exon indicates exon with stop codon. (FIG. 2B) Illustration showing deletion of exons 3-7 to generate ΔEx3-9. Sequences of gRNAs (SEQ ID NO: 1, SEQ ID NO: 4) and their targeting sites within intron 2 (top, SEQ ID NO: 101-102)) and intron 7 (bottom, SEQ ID NO: 104-105). gRNAs were designed to target 3′ region of intron 2 (gRNA-2) and 5′ region of intron 7 (gRNA-7). Arrowheads mark targeting site of gRNAs. PAM sites are highlighted in red. (FIG. 2C) PCR genotyping of control, ΔEx8-9 iDMD and two clones of ΔEx3-9 induced pluripotent cell (iPSC) lines using primers upstream and downstream of the gRNA targeting sites (top) and within intron 2 flanking the gRNA-2 targeting site (bottom). Sequencing of PCR product of ΔEx3-9 validates splicing of intron 2 to intron 7 (SEQ ID NO: 103). PCR primers are indicated by arrows. Arrowhead indicates gRNA targeting site. M denotes marker lane. (FIG. 2D) RT-PCR analysis of dystrophin mRNA expression in control, ΔEx8-9 iDMD, and two clones of ΔEx3-9 iPSC-derived cardiomyocytes. Forward primer targeting exon 1 and reverse primer targeting exon 10 were used. Sequencing confirmed splicing of exon 2 to exon 10 restoring the open reading frame (SEQ ID NO: 106-107). α-actinin was used as loading control. (FIG. 2E) Western blot analysis showing dystrophin protein expression in iPSC-derived cardiomyocytes using anti-dystrophin antibody. Vinculin was used as loading control. n=7 for control and ΔEx8-9 iDMD, n=3 for ΔEx3-9 clone 1 and ΔEx3-9 clone 2 n=7 for control and ΔEx8-9 iDMD, n=3 for ΔEx3-9 clone 1 and ΔEx3-9 clone 2. (FIG. 2F) Immunocytochemistry representations of iPSC-derived cardiomyocytes with anti-dystrophin (red) and anti-troponin I (green). Nuclei are stained with Hoechst 33342 (blue). Scale bar=50 microns. n=4 for control and ΔEx8-9 iDMD, n=2 for ΔEx3-9 clone 1, and n=1 for ΔEx3-9 clone 2.

FIG. 3A-E. Correcting ΔEx8-9 iDMD by deleting exons 6 and 7 to restore dystrophin protein expression. (FIG. 3A) Illustration showing deletion of exons 6-7 to generate ΔEx6-9. Sequences of guide RNAs (gRNAs) (SEQ ID NO: 2-3) and their targeting sites within intron 5 (top, SEQ ID NO: 108-109) and intron 7 (bottom, SEQ ID NO: 110-111). gRNAs were designed to target 3′ region of intron 5 (gRNA-5) and 5′ region of intron 7 (gRNA-7). Arrowheads mark targeting site of gRNAs. PAM sites are highlighted in red. (FIG. 3B) PCR genotyping of control, ΔEx8-9 iDMD and two clones of ΔEx6-9 induced pluripotent stem cell (iPSC) lines using primers upstream and downstream of the gRNA targeting sites (top) and within intron 5 flanking the gRNA-5 targeting site (bottom). Sequencing of PCR product of ΔEx6-9 validates splicing of intron 5 to intron 7 (SEQ ID NO: 112). PCR primers are indicated by arrows. Arrowhead indicates gRNA targeting site. M denotes marker lane. (FIG. 3C) RT-PCR analysis of dystrophin mRNA expression in control, ΔEx8-9 iDMD, and two clones of ΔEx6-9 iPSC-derived cardiomyocytes. Forward primer targeting exon 5 and reverse primer targeting exon 10 were used. Sequencing confirmed splicing of exon 5 to exon 10 restoring the open reading frame (SEQ ID NO: 113-114). α-actinin was used as loading control. (FIG. 3D) Western blot analysis showing dystrophin protein expression in iPSC-derived cardiomyocytes using anti-dystrophin antibody. Vinculin was used as loading control. n=7 for control and ΔEx8-9 iDMD, n=3 for ΔEx6-9 clone 1 and ΔEx6-9 clone 2. (FIG. 3E) Immunocytochemistry representations of iPSC-derived cardiomyocytes with anti-dystrophin (red) and anti-troponin I (green). Nuclei are stained with Hoechst 33342 (blue). Scale bar=50 microns. n=4 for control and ΔEx8-9 iDMD, n=3 for ΔEx6-9 clone 1 and ΔEx6-9 clone 2.

FIG. 4A-F. Correcting ΔEx8-9 iDMD by deleting exons 7-11 partially restores dystrophin protein expression. (FIG. 4A) Illustration showing deletion of exons 7-11 to generate ΔEx7-11. Sequences of guide RNAs (gRNAs) (SEQ ID NO: 115, SEQ ID NO: 118) and their targeting sites within intron 6 (top, SEQ ID NO: 116-117) and intron 11 (bottom, SEQ ID NO: 118-119). gRNAs were designed to target 3′ region of intron 6 (gRNA-6) and 5′ region of intron 11 (gRNA-11). Arrowheads mark gRNA targeting site. PAM sites are highlighted in red. (FIG. 4B) PCR genotyping of control, ΔEx8-9 iDMD and two clones of ΔEx7-11 induced pluripotent stem cell (iPSC) lines using primers upstream and downstream of the gRNA targeting sites (top) and within intron 6 flanking the gRNA-6 targeting site (bottom). Sequencing of PCR product of ΔEx7-11 validates splicing of intron 6 to intron 11 (SEQ ID NO: 121). PCR primers are indicated by arrows. Arrowhead indicates gRNA targeting site. M denotes marker lane. (FIG. 4C) RT-PCR analysis of dystrophin mRNA expression in control, ΔEx8-9 iDMD, and two clones of ΔEx7-11 iPSC-derived cardiomyocytes. Forward primer targeting exon 5 and reverse primer targeting exon 12 were used. Sequencing confirmed splicing of exon 6 to exon 12 restoring the open reading frame (SEQ ID NO: 122-123). α-actinin was used as loading control. (FIG. 4D) Western blot analysis showing dystrophin protein expression in iPSC-derived cardiomyocytes using anti-dystrophin antibody. Vinculin was used as loading control. n=7 for control and ΔEx8-9 iDMD, n=3 for ΔEx7-11 clone 1 and ΔEx7-11 clone 2. (FIG. 4E) Immunocytochemistry representations of iPSC-derived cardiomyocytes with anti-dystrophin (red) and anti-troponin I (green). Nuclei are stained with Hoechst 33342 (blue). Scale bar=50 microns. n=4 for control and ΔEx8-9 iDMD, n=2 for ΔEx7-11 clone 1 and n=1 for ΔEx7-11 clone 2. (FIG. 4F) Western blot analysis of ΔEx7-11 iPSC-derived cardiomyocytes treated with proteasome inhibitor MG132 for 60 hours using anti-dystrophin antibody. GAPDH was used as loading control. n=2.

FIG. 5A-G. Functional analysis of iPSC-derived cardiomyocytes. (FIG. 5A) Representative recordings of spontaneous Ca²⁺ activity of induced pluripotent cell (iPSC)-derived cardiomyocytes loaded with Ca²⁺ indicator Fluo-4AM. Traces show change in fluorescence intensity (F) in relationship to resting fluorescence intensity (Fo). (FIG. 5B) Relative time to peak (TTP), (FIG. 5C) decay (tau) and (FIG. 5D) transient duration (TD), as measured by calcium imaging. (FIG. 5E) Arrhythmic iPSC-derived cardiomyocytes were identified based on calcium activity. For all corrected iPSC-derived cardiomyocyte lines (panels B-E), data were obtained from two independent clones. n=113 for control; n=105 for ΔEx8-9 iDMD; n=129 for ΔEx3-9; n=121 for ΔEx6-9; and n=122 for ΔEx7-11 iPSC-derived cardiomyocyte lines from 5-11 independent experiments. Data are represented as mean+s.e.m. *P<0.05 by one-way ANOVA. (FIG. 5F) Force of EHM contraction in the presence of increasing Ca²⁺ concentration. EHM generated from two independent control lines (n=8), ΔEx8-9 iDMD (n=6), ΔEx3-9 (n=14), ΔEx6-9 (n=3), and ΔEx7-11 (n=3). (FIG. 5G) Representative recordings of EHM contractions from the indicated groups (same EHM as in FIG. 5F). Data are represented as mean+s.e.m. *P<0.05 by two-way ANOVA and Tukey's post-hoc test.

FIG. 6A-I Correction of DMD patient-derived iPSCs by deleting exons 8 and 9. (FIG. 6A) Strategy showing CRISPR/Cas9-mediated genomic editing of DMD (Duchene muscular dystrophy) patient (pΔEx3-7) to generate corrected pΔEx3-9 induced pluripotent cell (iPSC) line. Shape and color of boxes denoting DMD exons indicate reading frame and protein coding domains. Yellow designates ABD-1. Blue marks part of central rod domain. Red lines indicate ABS 1. Arrowheads mark targeting site of guide RNAs (gRNAs). Red box marks exon with stop codon. Sequences of gRNAs (SEQ ID NO: 124, SEQ ID NO: 5) and their targeting sites within intron 7 (top, SEQ ID NO: 125-126) and intron 9 (bottom, SEQ ID NO: 128-129). gRNAs were designed to target 3′ region of intron 7 (gRNA-7) and 5′ region of intron 9 (gRNA-9). PAM sites are highlighted in red. (FIG. 6B) PCR genotyping of pΔEx3-7 and pΔEx3-9 iPSC lines using primers upstream and downstream of the gRNA targeting sites. Sequencing of PCR product of pΔEx3-9 validates splicing of intron 7 to intron 9 (SEQ ID NO: 127). PCR primers are indicated by arrows. Arrowheads mark targeting site of gRNAs. M denotes marker lane. (FIG. 6C) RT-PCR analysis of dystrophin mRNA expression in control, pΔEx3-7, pΔEx3-9 iPSC-derived cardiomyocytes. Forward primer targeting 5′UTR and reverse primer targeting exon 10 were used. Sequencing of pΔEx3-7 confirmed splicing of exon 2 to exon 8 (SEQ ID NO: 130-131), introducing a stop-codon. Sequencing pΔEx3-9 confirmed splicing of exon 2 to exon 10 (SEQ ID NO: 132-133) restoring the open reading frame. α-actinin was used as loading control. (FIG. 6D) Western blot analysis showing dystrophin protein expression in iPSC-derived cardiomyocytes using anti-dystrophin antibody. Vinculin was used as loading control. n=7 for control, n=3 for pΔEx3-7 and n=3 for pΔEx3-9. (FIG. 6E) Immunocytochemistry representations of iPSC-derived cardiomyocytes with anti-dystrophin (red) and anti-troponin I (green). Nuclei are stained with Hoechst 33342 (blue). Scale bar=50 microns. n=4 for control, n=2 for pΔEx3-7 and n=2 for pΔEx3-9. (FIG. 6F) Relative time to peak (TTP), (FIG. 6G) decay (tau), (FIG. 6H) transient duration (TD) and (FIG. 6I) percent of arrhythmic cells were measured based on calcium activity of control (n=45), DMD patient pΔEx3-7 (n=40) and corrected pΔEx3-9 (n=65) iPSC-derived cardiomyocytes from 3-5 independent experiments. Data are represented as mean+s.e.m. *P<0.05 by one-way ANOVA (FIGS. 6F-H).

FIG. 7A-C. Dystrophin protein expression level. Related to FIGS. 3A-E and FIGS. 4A-F. (FIG. 7A) Dystrophin expression levels in ΔEx8-9 iDMD (n=8), ΔEx3-9 (n=4), ΔEx6-9 (n=6), and ΔEx7-11 (n=5) iPSC-derived cardiomyocytes compared to control cell line, indicated by dashed line. Data are represented as mean±s.e.m. *P<0.05. (FIG. 7B) Western blot analysis of dystrophin (top), Myosin heavy chain, Myh, (middle) and GAPDH (bottom) expression in control, ΔEx8-9 iDMD, ΔEx3-9, ΔEx6-9, and ΔEx7-11 iPSC-derived cardiomyocytes. Arrowhead indicates residual dystrophin protein after stripping the blot. Arrow indicates myosin heavy chain expression. (FIG. 7C) Western blot analysis of ΔEx7-11 clone 2 iPSC-derived cardiomyocytes treated with proteasome inhibitor MG132 for 60 hours using anti-dystrophin antibody. GAPDH and Vinculin were used as loading control.

FIG. 8A-C. Generation and functional analysis of engineered heart muscle (EHM). Related to FIGS. 5A-G. (FIG. 8A) Schematic diagram of EHM generation. (FIG. 8B) Percentage of α-actinin-positive (ACTN2+) cardiomyocytes before EHM generation. n=7 total EHM analyzed. (FIG. 8C) Percentage of EHM arrhythmic contractions.

FIG. 9A-E. Generation of DMD mouse model by deletion of Dmd exons 8 and 9. (FIG. 9A) Outline of the CRISPR/Cas9 strategy used to generate the DMD mouse model, ΔEx8-9 by excising exons 8 and 9. (FIG. 9B) Hematoxylin and eosin (H&E) immunostaining of quadriceps, diaphragm and heart in wild type (WT) and ΔEx8-9 DMD mice. (FIG. 9C) Dystrophin immunostaining of quadriceps, diaphragm and heart of wild type (WT) and ΔEx8-9 DMD mice. Dystrophin immunostains in red. Nucleus are marked by DAPI stain in blue. (FIG. 9D) Grip strength measured in WT and ΔEx8-9 DMD mice. (FIG. 9E) Serum creatine kinase (CK), a marker of muscle dystrophy that reflects muscle damage and membrane leakage was measured in WT and ΔEx8-9 DMD mice.

### SEQUENCE TABLES

TABLE 1
Human DMD guide RNA for actin binding domain region

| guide | | | PAM |
|---|---|---|---|
| RNAs | DNA sequence | RNA sequence | site |
| intron 2-3′ | AATTAATCTGCCGAAGATGA | AAUUAAUCUGCCGAAGAUGA | CGG |
| | (SEQ ID NO: 1) | (SEQ ID NO: 6) | |
| intron 5-3′ | AAAAGCGCTTTTTGGATAGG | AAAAGCGCUUUUUGGAUAGG | AGG |
| | (SEQ ID NO: 2) | (SEQ ID NO: 7) | |
| intron 7-5′ | TGCAGTATGCTCCATCCATA | UGCAGUAUGCUCCAUCCAUA | GGG |
| | (SEQ ID NO: 3) | (SEQ ID NO: 8) | |
| intron 7-3′ | TGTCAATTCAAATGGTGCAC | UGUCAAUUCAAAUGGUGCAC | TGG |
| | (SEQ ID NO: 4) | (SEQ ID NO: 9) | |
| intron 9-5′ | AAACAAACCAGCTCTTCACG | AAACAAACCAGCUCUUCACG | AGG |
| | (SEQ ID NO: 5) | (SEQ ID NO: 10) | |

TABLE 2
Mouse DMD guide RNA for actin binding domain region

| guide | | | PAM |
|---|---|---|---|
| RNAs | DNA sequence | RNA sequence | site |
| intron 7-3′ | TAGTCTCTAGAGGACGTTCA | UAGUCUCUAGAGGACGUUCA | TGG |
| | (SEQ ID NO: 11) | (SEQ ID NO: 13) | |
| intron 9-5′ | TGTTGTCATGAGGCCTATCT | UGUUGUCAUGAGGCCUAUCU | TGG |
| | (SEQ ID NO: 12) | (SEQ ID NO: 14) | |

### DETAILED DESCRIPTION

DMD is associated with greater than 4,000 mutations in the DMD gene, which are comprised primarily of exon deletions, as well as exon duplications and small mutations that include point mutations. DMD mutations cluster into two hot spot regions of the gene. One hot spot is located within the 5′ region of the gene, encompassing exons 2-20. These mutations account for ˜15% of all exon deletions and ˜50% of all exon duplications within the DMD gene. Deletion of exons 3-7 are the most frequent. The second DMD hot spot is located in the distal region of the DMD gene, between exons 45-55, accounting for ˜70% of all exon deletions and ˜15% of all exon duplications. Because a majority of patient mutations carry deletions in these hotspots, a therapeutic approach for skipping certain exon applies to large group of patients. The rationale of the exon skipping approach is based on the genetic difference between DMD and Becker muscular dystrophy (BMD) patients. In DMD patients, the reading frame of dystrophin mRNA is disrupted resulting in prematurely truncated, non-functional dystrophin proteins. BMD patients have mutations in the DMD gene that maintain the reading frame allowing the production of internally deleted, but partially functional dystrophins leading to much milder disease symptoms compared to DMD patients.

Dystrophin protein consists of 3,685 amino acids and can be separated into four domains: 1) the actin binding domain (ABD-1), which consists of amino acids 14-240 and connects the filamentous elements of the cytoskeleton to the cell membrane; 2) the central rod domain, composed of 24 spectrin-like repeats and the second actin-binding domain (ABD-2); 3) the cysteine-rich domain; and 4) the carboxyl-terminal domain. Together, these four domains provide the function of dystrophin as a structural link between the cytoskeleton and extracellular matrix to maintain muscle integrity.

The dystrophin protein can tolerate internal deletions that maintain a subset of the rod domains with intact amino- and carboxyl-termini regions, resulting in mild loss of muscle function, as seen in patients with BMD. There have been numerous strategies to convert DMD to BMD by skipping or deleting out-of-frame exons and restoring the expression of truncated forms of dystrophin protein. Similarly, shortened forms of dystrophin, referred to as mini- or micro-dystrophins, are being developed for gene therapy. It should be noted that the precise consequences of in-frame deletions on the stability and function of dystrophin are not predictable a priori, as some in-frame deletions cause severe disease while others have only mild effects. Thus, it is important to analyze the dystrophin protein products generated from in-frame deletions before reaching conclusions regarding their potential therapeutic effects.

Previously, the inventors and others used CRISPR/Cas9-mediated genome editing to permanently correct dystrophin mutations in mouse models of DMD and patient-derived muscle cells. These efforts focused mainly on correcting mutations in the spectrin-like repeat region to restore dystrophin function by generating truncated dystrophin protein, similar to the forms associated with BMD. The ABD-1 of dystrophin contains three actin-binding sites (ABS1-3) that associate with F-actin and are essential for the stabilization of muscle membranes by dystrophin. Little emphasis has been put on editing the ABD-1 region of the DMD gene, although ˜7% of DMD patients have mutations in the ABD-1 domain. In fact, exons 2-7, which encode part of the ABD-1, are the most frequently mutated portion of the 5′-proximal hot spot. In-frame deletions and missense mutations of the 5′ region of the DMD gene that affect the ABD-1 structure have been associated with a decrease in dystrophin protein stability, reduced actin binding affinity, and protein mis-folding and degradation, suggesting that restoring the open reading frame of ABD-1 mutations by genome editing strategies may not be sufficient to correct DMD. On the other hand, medical case studies have reported patients with deletions in exons 3-9 in the DMD gene that exhibit no apparent phenotype, suggesting that precise deletion of exons 3-9 may be an effective approach to correct mutations in the ABD-1 region. Accordingly, uncertainty remains regarding whether gene editing in this region is or is not corrective.

As described in further detail below, the instant inventors demonstrate that gene editing in the actin-binding domain region of the dystrophin gene is a viable method for treating DMD.

Human induced pluripotent stem cells (iPSCs) offer a unique opportunity for studying human diseases in a dish. Their unlimited proliferation, ability to undergo clonal selection, and differentiation into different cell types provide a reliable platform for testing gene editing strategies to either create or correct human mutations. Differentiation of DMD-derived iPSCs to cardiomyocytes prior to and following genome editing allows the analysis of DMD phenotypes by assessing dystrophin expression and cardiomyocyte function, such as muscle contractility and Ca²⁺ handling. Assembly of iPSC-derived cardiomyocytes as engineered heart muscle (EHM) allows for direct and controlled measurements of heart muscle force of contraction.

Here the inventors introduced an exon 8-9 deletion (ΔEx8-9) in the DMD gene of healthy donor-derived iPSCs to generate a DMD iPSC line. This ΔEx8-9 DMD iPSC line allowed correction of the DMD mutation by various gene editing strategies and then assess truncated dystrophin functionality in comparison to isogenic control cells. They corrected the exon 8-9 deletion mutation using three different exon-deletion strategies to restore the open reading frames: 1) deleting exons 3-7 to generate ΔEx3-9, which excises the ABS-2 and ABS-3 regions; 2) deleting exons 6-7 to generate ΔEx6-9, which excises ABS-3; and 3) deleting exons 7-11 to generate ΔEx7-11, which leaves all three ABS regions intact. The inventors show that deletion DMD exons 3-9 generates a truncated dystrophin but maintains the structure of dystrophin such that it restores cardiomyocyte functionality. These findings provide a promising strategy for correction of DMD mutations within the proximal hot spot of the DMD gene by genomic editing, allowing restoration of dystrophin expression and function. These and other aspects of the disclosure are reproduced below.

To further assess the efficiency and optimize CRISPR/Cas9-mediated exon skipping in vivo, a mimic of the human ABD-1 region mutation was generated in a mouse model by deleting the exons 8 and 9 using CRISPR/Cas9 system directed by two single guide RNAs (sgRNAs). The ΔEx8-9 DMD mouse model exhibits dystrophic myofibers, increased serum creatine kinase level, and reduced muscle function, thus providing a new representative model of DMD. These and other aspects of the disclosure are reproduced below.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, for the method being employed to determine the value, or that exists among the study subjects. Such an inherent variation may be a variation of ±10% of the stated value.

### I. DUCHENNE MUSCULAR DYSTROPHY

A. Background

Duchenne muscular dystrophy (DMD) is a recessive X-linked form of muscular dystrophy, affecting around 1 in 5000 boys, which results in muscle degeneration and premature death. The disorder is caused by a mutation in the gene dystrophin (see GenBank Accession No. NC_000023.11), located on the human X chromosome, which codes for the protein dystrophin (GenBank Accession No. AAA53189; SEQ ID NO. 15), the sequence of which is reproduced below:

| | |
|---|---|
| 1 | MLWWEEVEDC YEREDVQKKT FTKWVNAQFS KFGKQHIENL FSDLQDGRRL LDLLEGLTGQ |
| 61 | KLPKEKGSTR VHALNNVNKA LRVLQNNNVD LVNIGSTDIV DGNHKLTLGL IWNIILHWQV |
| 121 | KNVMKNIMAG LQQTNSEKIL LSWVRQSTRN YPQVNVINFT TSWSDGLALN ALIHSHRPDL |
| 181 | FDWNSVVCQQ SATQRLEHAF NIARYQLGIE KLLDPEDVDT TYPDKKSILM YITSLFQVLP |
| 241 | QQVSIEAIQE VEMLPRPPKV TKEEHFQLHH QMHYSQQITV SLAQGYERTS SPKPRFKSYA |
| 301 | YTQAAYVTTS DPTRSPFPSQ HLEAPEDKSF GSSLMESEVN LDRYQTALEE VLSWLLSAED |
| 361 | TLQAQGEISN DVEVVKDQFH THEGYMMDLT AHQGRVGNIL QLGSKLIGTG KLSEDEETEV |
| 421 | QEQMNLLNSR WECLRVASME KQSNLHRVLM DLQNQKLKEL NDWLTKTEER TRKMEEEPLG |
| 481 | PDLEDLKRQV QQHKVLQEDL EQEQVRVNSL THMVVVVDES SGDHATAALE EQLKVLGDRW |
| 541 | ANICRWTEDR WVLLQDILLK WQRLTEEQCL FSAWLSEKED AVNKIHTTGF KDQNEMLSSL |
| 601 | QKLAVLKADL EKKKQSMGKL YSLKQDLLST LKNKSVTQKT EAWLDNFARC WDNLVQKLEK |
| 661 | STAQISQAVT TTQPSLTQTT VMETVTTVTT REQILVKHAQ EELPPPPPQK KRQITVDSEI |
| 721 | RKRLDVDITE LHSWITRSEA VLQSPEFAIF RKEGNFSDLK EKVNAIEREK AEKFRKLQDA |
| 781 | SRSAQALVEQ MVNEGVNADS IKQASEQLNS RWIEFCQLLS ERLNWLEYQN NIIAFYNQLQ |
| 841 | QLEQMTTTAE NWLKIQPTTP SEPTAIKSQL KICKDEVNRL SGLQPQIERL KIQSIALKEK |
| 901 | GQGPMFLDAD FVAFTNHFKQ VFSDVQAREK ELQTIFDTLP PMRYQETMSA IRTWVQQSET |
| 961 | KLSIPQLSVT DYEIMEQRLG ELQALQSSLQ EQQSGLYYLS TTVKEMSKKA PSEISRKYQS |
| 1021 | EFEEIEGRWK KLSSQLVEHC QKLEEQMNKL RKIQNHIQTL KKWMAEVDVF LKEEWPALGD |
| 1081 | SEILKKQLKQ CRLLVSDIQT IQPSLNSVNE GGQKIKNEAE PEFASRLETE LKELNTQWDH |
| 1141 | MCQQVYARKE ALKGGLEKTV SLQKDLSEMH EWMTQAEEEY LERDFEYKTP DELQKAVEEM |
| 1201 | KRAKEEAQQK EAKVKLLTES VNSVIAQAPP VAQEALKKEL ETLTTNYQWL CTRLNGKCKT |
| 1261 | LEEVWACWHE LLSYLEKANK WLNEVEFKLK TTENIPGGAE EISEVLDSLE NLMRHSEDNP |
| 1321 | NQIRILAQTL TDGGVMDELI NEELETFNSR WRELHEEAVR RQKLLEQSIQ SAQETEKSLH |
| 1381 | LIQESLTFID KQLAAYIADK VDAAQMPQEA QKIQSDLTSH EISLEEMKKH NQGKEAAQRV |
| 1441 | LSQIDVAQKK LQDVSMKFRL FQKPANFELR LQESKMILDE VKMHLPALET KSVEQEVVQS |
| 1501 | QLNHCVNLYK SLSEVKSEVE MVIKTGRQIV QKKQTENPKE LDERVTALKL HYNELGAKVT |
| 1561 | ERKQQLEKCL KLSRKMRKEM NVLTEWLAAT DMELTKRSAV EGMPSNLDSE VAWGKATQKE |
| 1621 | IEKQKVHLKS ITEVGEALKT VLGKKETLVE DKLSLLNSNW IAVTSRAEEW LNLLLEYQKH |
| 1681 | METFDQNVDH ITKWIIQADT LLDESEKKKP QQKEDVLKRL KAELNDIRPK VDSTRDQAAN |
| 1741 | LMANRGDHCR KLVEPQISEL NHRFAAISHR IKTGKASIPL KELEQFNSDI QKLLEPLEAE |
| 1801 | IQQGVNLKEE DFNKDMNEDN EGTVKELLQR GDNLQQRITD ERKREEIKIK QQLLQTKHNA |
| 1861 | LKDLRSQRRK KALEISHQWY QYKRQADDLL KCLDDIEKKL ASLPEPRDER KIKEIDRELQ |
| 1921 | KKKEELNAVR RQAEGLSEDG AAMAVEPTQI QLSKRWREIE SKFAQFRRLN FAQIHTVREE |
| 1981 | TMMVMTEDMP LEISYVPSTY LTEITHVSQA LLEVEQLLNA PDLCAKDFED LFKQEESLKN |
| 2041 | IKDSLQQSSG RIDIIHSKKT AALQSATPVE RVKLQEALSQ LDFQWEKVNK MYKDRQGRFD |
| 2101 | RSVEKWRRFH YDIKIFNQWL TEAEQFLRKT QIPENWEHAK YKWYLKELQD GIGQRQTVVR |
| 2161 | TLNATGEEII QQSSKTDASI LQEKLGSLNL RWQEVCKQLS DRKKRLEEQK NILSEFQRDL |
| 2221 | NEFVLWLEEA DNIASIPLEP GKEQQLKEKL EQVKLLVEEL PLRQGILKQL NETGGPVLVS |
| 2281 | APISPEEQDK LENKLKQTNL QWIKVSRALP EKQGEIEAQI KDLGQLEKKL EDLEEQLNHL |
| 2341 | LLWLSPIRNQ LEIYNQPNQE GPFDVQETEI AVQAKQPDVE EILSKGQHLY KEKPATQPVK |
| 2401 | RKLEDLSSEW KAVNRLLQEL RAKQPDLAPG LTTIGASPTQ TVTLVTQPVV TKETAISKLE |
| 2461 | MPSSLMLEVP ALADFNRAWT ELTDWLSLLD QVIKSQRVMV GDLEDINEMI IKQKATMQDL |
| 2521 | EQRRPQLEEL ITAAQNLKNK TSNQEARTII TDRIERIQNQ WDEVQEHLQN RRQQLNEMLK |
| 2581 | DSTQWLEAKE EAEQVLGQAR AKLESWKEGP YTVDAIQKKI TETKQLAKDL RQWQTNVDVA |
| 2641 | NDLALKLLRD YSADDTRKVH MITENINASW RSIHKRVSER EAALEETHRL LQQFPLDLEK |
| 2701 | FLAWLTEAET TANVLQDATR KERLLEDSKG VKELMKQWQD LQGEIEAHTD VYHNLDENSQ |
| 2761 | KILRSLEGSD DAVLLQRRLD NMNFKWSELR KKSLNIRSHL EASSDQWKRL HLSLQELLVW |
| 2821 | LQLKDDELSR QAPIGGDFPA VQKQNDVHRA FKRELKTKEP VIMSTLETVR IFLTEQPLEG |
| 2881 | LEKLYQEPRE LPPEERAQNV TRLLRKQAEE VNTEWEKLNL HSADWQRKID ETLERLQELQ |
| 2941 | EATDELDLKL RQAEVIKGSW QPVGDLLIDS LQDHLEKVKA LRGEIAPLKE NVSHVNDLAR |
| 3001 | QLTTLGIQLS PYNLSTLEDL NTRWKLLQVA VEDRVRQLHE AHRDFGPASQ HFLSTSVQGP |
| 3061 | WERAISPNKV PYYINHETQT TCWDHPKMTE LYQSLADLNN VRFSAYRTAM KLRRLQKALC |
| 3121 | LDLLSLSAAC DALDQHNLKQ NDQPMDILQI INCLTTIYDR LEQEHNNLVN VPLCVDMCLN |
| 3181 | WLLNVYDTGR TGRIRVLSFK TGIISLCKAH LEDKYRYLFK QVASSTGFCD QRRLGLLLHD |
| 3241 | SIQIPRQLGE VASFGGSNIE PSVRSCFQFA NNKPEIEAAL FLDWMRLEPQ SMVWLPVLHR |
| 3301 | VAAAETAKHQ AKCNICKECP IIGFRYRSLK HFNYDICQSC FFSGRVAKGH KMHYPMVEYC |
| 3361 | TPTTSGEDVR DFAKVLKNKF RTKRYFAKHP RMGYLPVQTV LEGDNMETPV TLINFWPVDS |
| 3421 | APASSPQLSH DDTHSRIEHY ASRLAEMENS NGSYLNDSIS PNESIDDEHL LIQHYCQSLN |
| 3481 | QDSPLSQPRS PAQILISLES EERGELERIL ADLEEENRNL QAEYDRLKQQ HEHKGLSPLP |
| 3541 | SPPEMMPTSP QSPRDAELIA EAKLLRQHKG RLEARMQILE DHNKQLESQL HRLRQLLEQP |
| 3601 | QAEAKVNGTT VSSPSTSLQR SDSSQPMLLR VVGSQTSDSM GEEDLLSPPQ DTSTGLEEVM |
| 3661 | EQLNNSFPSS RGRNTPGKPM REDTM |

In humans, dystrophin mRNA contains 79 exons. Dystrophin mRNA is known to be alternatively spliced, resulting in various isoforms. Exemplary dystrophin isoforms are listed in Table 3.

TABLE 3
Dystrophin isoforms

| | | Nucleic | | | |
|---|---|---|---|---|---|
| | | Acid | | Protein | |
| Sequence | Nucleic Acid | SEQ ID | Protein Accession | SEQ | Description |
| Name | Accession No. | NO: | No. | NO: | ID |
| DMD Genomic | NC_000023.11 | None | None | None | Sequence from Human |
| Sequence | (positions | | | | X Chromosome (at |
| | 31119219 to | | | | positions Xp21.2 to |
| | 33339609) | | | | p21.1) from Assembly |
| | | | | | GRCh38.p7 |
| | | | | | (GCF_000001405.33) |
| Dystrophin | NM_000109.3 | 16 | NP_000100.2 | 17 | Transcript Variant: |
| Dp427c isoform | | | | | transcript Dp427c is |
| | | | | | expressed |
| | | | | | predominantly in |
| | | | | | neurons of the cortex |
| | | | | | and the CA regions of |
| | | | | | the hippocampus. It |
| | | | | | uses a unique |
| | | | | | promoter/exon 1 |
| | | | | | located about 130 kb |
| | | | | | upstream of the |
| | | | | | Dp427m transcript |
| | | | | | promoter. The |
| | | | | | transcript includes the |
| | | | | | common exon 2 of |
| | | | | | transcript Dp427m and |
| | | | | | has a similar length of |
| | | | | | 14 kb. The Dp427c |
| | | | | | isoform contains a |
| | | | | | unique N-terminal |
| | | | | | MED sequence, instead |
| | | | | | of the |
| | | | | | MLWWEEVEDCY |
| | | | | | sequence of isoform |
| | | | | | Dp427m. The |
| | | | | | remainder of isoform |
| | | | | | Dp427c is identical to |
| | | | | | isoform Dp427m. |
| Dystrophin | NM_004006.2 | 18 | NP_003997.1 | 19 | Transcript Variant: |
| Dp427m | | | | | transcript Dp427m |
| isoform | | | | | encodes the main |
| | | | | | dystrophin protein |
| | | | | | found in muscle. As a |
| | | | | | result of alternative |
| | | | | | promoter use, exon 1 |
| | | | | | encodes a unique N- |
| | | | | | terminal |
| | | | | | MLWWEEVEDCY aa |
| | | | | | sequence. |
| Dystrophin | NM_004009.3 | 20 | NP_004000.1 | 21 | Transcript Variant: |
| Dp427p1 | | | | | transcript Dp427p1 |
| isoform | | | | | initiates from a unique |
| | | | | | promoter/exon 1 |
| | | | | | located in what |
| | | | | | corresponds to the first |
| | | | | | intron of transcript |
| | | | | | Dp427m. The transcript |
| | | | | | adds the common exon |
| | | | | | 2 of Dp427m and has a |
| | | | | | similar length (14 kb). |
| | | | | | The Dp427p1 isoform |
| | | | | | replaces the |
| | | | | | MLWWEEVEDCY - |
| | | | | | start of Dp427m with a |
| | | | | | unique N-terminal |
| | | | | | MSEVSSD aa |
| | | | | | sequence. |
| Dystrophin | NM_004011.3 | 22 | NP_004002.2 | 23 | Transcript Variant: |
| Dp260-1 | | | | | transcript Dp260-1 uses |
| isoform | | | | | exons 30-79, and |
| | | | | | originates from a |
| | | | | | promoter/exon 1 |
| | | | | | sequence located in |
| | | | | | intron 29 of the |
| | | | | | dystrophin gene. As a |
| | | | | | result, Dp260-1 |
| | | | | | contains a 95 bp exon 1 |
| | | | | | encoding a unique N- |
| | | | | | terminal 16 aa |
| | | | | | MTEIILLIFFPAYFLN- |
| | | | | | sequence that replaces |
| | | | | | amino acids 1-1357 of |
| | | | | | the full-length |
| | | | | | dystrophin product |
| | | | | | (Dp427m isoform). |
| Dystrophin | NM_004012.3 | 24 | NP_004003.1 | 25 | Transcript Variant: |
| Dp260-2 | | | | | transcript Dp260-2 uses |
| isoform | | | | | exons 30-79, starting |
| | | | | | from a promoter/exon 1 |
| | | | | | sequence located in |
| | | | | | intron 29 of the |
| | | | | | dystrophin gene that is |
| | | | | | alternatively spliced |
| | | | | | and lacks N-terminal |
| | | | | | amino acids 1-1357 of |
| | | | | | the full length |
| | | | | | dystrophin (Dp427m |
| | | | | | isoform). The Dp260-2 |
| | | | | | transcript encodes a |
| | | | | | unique N-terminal |
| | | | | | MSARKLRNLSYKK |
| | | | | | sequence. |
| Dystrophin | NM_004013.2 | 26 | NP_004004.1 | 27 | Transcript Variant: |
| Dp140 isoform | | | | | Dp140 transcripts use |
| | | | | | exons 45-79, starting at |
| | | | | | a promoter/exon 1 |
| | | | | | located in intron 44. |
| | | | | | Dp140 transcripts have |
| | | | | | along (1 kb) 5′ UTR |
| | | | | | since translation is |
| | | | | | initiated in exon 51 |
| | | | | | (corresponding to aa |
| | | | | | 2461 of dystrophin). In |
| | | | | | addition to the |
| | | | | | alternative promoter |
| | | | | | and exon 1, differential |
| | | | | | splicing of exons 71-74 |
| | | | | | and 78 produces at least |
| | | | | | five Dp140 isoforms. |
| | | | | | Of these, this transcript |
| | | | | | (Dp140) contains all of |
| | | | | | the exons. |
| Dystrophin | NM_004014.2 | 28 | NP_004005.1 | 29 | Transcript Variant: |
| Dp116 isoform | | | | | transcript Dp116 uses |
| | | | | | exons 56-79, starting |
| | | | | | from a promoter/exon 1 |
| | | | | | within intron 55. As a |
| | | | | | result, the Dp116 |
| | | | | | isoform contains a |
| | | | | | unique N-terminal |
| | | | | | MLHRKTYHVK aa |
| | | | | | sequence, instead of aa |
| | | | | | 1-2739 of dystrophin. |
| | | | | | Differential splicing |
| | | | | | produces several |
| | | | | | Dp116-subtypes. The |
| | | | | | Dp116 isoform is also |
| | | | | | known as S-dystrophin |
| | | | | | or apo-dystrophin-2. |
| Dystrophin | NM_004015.2 | 30 | NP_004006.1 | 31 | Transcript Variant: |
| Dp71 isoform | | | | | Dp71 transcripts use |
| | | | | | exons 63-79 with a |
| | | | | | novel 80- to 100-nt |
| | | | | | exon containing an |
| | | | | | ATG start site for a |
| | | | | | new coding sequence of |
| | | | | | 17 nt. The short coding |
| | | | | | sequence is in-frame |
| | | | | | with the consecutive |
| | | | | | dystrophin sequence |
| | | | | | from exon 63. |
| | | | | | Differential splicing of |
| | | | | | exons 71 and 78 |
| | | | | | produces at least four |
| | | | | | Dp71 isoforms. Of |
| | | | | | these, this transcript |
| | | | | | (Dp71) includes both |
| | | | | | exons 71 and 78. |
| Dystrophin | NM_004016.2 | 32 | NP_004007.1 | 33 | Transcript Variant: |
| Dp71b isoform | | | | | Dp71 transcripts use |
| | | | | | exons 63-79 with a |
| | | | | | novel 80- to 100-nt |
| | | | | | exon containing an |
| | | | | | ATG start site for a |
| | | | | | new coding sequence of |
| | | | | | 17 nt. The short coding |
| | | | | | sequence is in-frame |
| | | | | | with the consecutive |
| | | | | | dystrophin sequence |
| | | | | | from exon 63. |
| | | | | | Differential splicing of |
| | | | | | exons 71 and 78 |
| | | | | | produces at least four |
| | | | | | Dp71 isoforms. Of |
| | | | | | these, this transcript |
| | | | | | (Dp71b) lacks exon 78 |
| | | | | | and encodes a protein |
| | | | | | with a different C- |
| | | | | | terminus than Dp71 and |
| | | | | | Dp71a isoforms. |
| Dystrophin | NM_004017.2 | 34 | NP_004008.1 | 35 | Transcript Variant: |
| Dp71a isoform | | | | | Dp71 transcripts use |
| | | | | | exons 63-79 with a |
| | | | | | novel 80- to 100-nt |
| | | | | | exon containing an |
| | | | | | ATG start site for a |
| | | | | | new coding sequence of |
| | | | | | 17 nt. The short coding |
| | | | | | sequence is in-frame |
| | | | | | with the consecutive |
| | | | | | dystrophin sequence |
| | | | | | from exon 63. |
| | | | | | Differential splicing of |
| | | | | | exons 71 and 78 |
| | | | | | produces at least four |
| | | | | | Dp71 isoforms. Of |
| | | | | | these, this transcript |
| | | | | | (Dp71a) lacks exon 71. |
| Dystrophin | NM_004018.2 | 36 | NP_004009.1 | 37 | Transcript Variant: |
| Dp71ab isoform | | | | | Dp71 transcripts use |
| | | | | | exons 63-79 with a |
| | | | | | novel 80- to 100-nt |
| | | | | | exon containing an |
| | | | | | ATG start site for a |
| | | | | | new coding sequence of |
| | | | | | 17 nt. The short coding |
| | | | | | sequence is in-frame |
| | | | | | with the consecutive |
| | | | | | dystrophin sequence |
| | | | | | from exon 63. |
| | | | | | Differential splicing of |
| | | | | | exons 71 and 78 |
| | | | | | produces at least four |
| | | | | | Dp71 isoforms. Of |
| | | | | | these, this transcript |
| | | | | | (Dp71ab) lacks both |
| | | | | | exons 71 and 78 and |
| | | | | | encodes a protein with |
| | | | | | a C-terminus like |
| | | | | | isoform Dp71b. |
| Dystrophin | NM_004019.2 | 38 | NP_004010.1 | 39 | Transcript Variant: |
| Dp40 isoform | | | | | transcript Dp40 uses |
| | | | | | exons 63-70. The 5′ |
| | | | | | UTR and encoded first |
| | | | | | 7 aa are identical to that |
| | | | | | in transcript Dp71, but |
| | | | | | the stop codon lies at |
| | | | | | the splice junction of |
| | | | | | the exon/intron 70. The |
| | | | | | 3′ UTR includes nt |
| | | | | | from intron 70 which |
| | | | | | includes an alternative |
| | | | | | polyadenylation site. |
| | | | | | The Dp40 isoform |
| | | | | | lacks the normal C- |
| | | | | | terminal end of full- |
| | | | | | length dystrophin (aa |
| | | | | | 3409-3685). |
| Dystrophin | NM_004020.3 | 40 | NP_004011.2 | 41 | Transcript Variant: |
| Dp140c isoform | | | | | Dp140 transcripts use |
| | | | | | exons 45-79, starting at |
| | | | | | a promoter/exon 1 |
| | | | | | located in intron 44. |
| | | | | | Dp140 transcripts have |
| | | | | | along (1 kb) 5′ UTR |
| | | | | | since translation is |
| | | | | | initiated in exon 51 |
| | | | | | (corresponding to aa |
| | | | | | 2461 of dystrophin). In |
| | | | | | addition to the |
| | | | | | alternative promoter |
| | | | | | and exon 1, differential |
| | | | | | splicing of exons 71-74 |
| | | | | | and 78 produces at least |
| | | | | | five Dp140 isoforms. |
| | | | | | Of these, this transcript |
| | | | | | (Dp140c) lacks exons |
| | | | | | 71-74. |
| Dystrophin | NM_004021.2 | 42 | NP_004012.1 | 43 | Transcript Variant: |
| Dp140b | | | | | Dp140 transcripts use |
| isoform | | | | | exons 45-79, starting at |
| | | | | | a promoter/exon 1 |
| | | | | | located in intron 44. |
| | | | | | Dp140 transcripts have |
| | | | | | along (1 kb) 5′ UTR |
| | | | | | since translation is |
| | | | | | initiated in exon 51 |
| | | | | | (corresponding to aa |
| | | | | | 2461 of dystrophin). In |
| | | | | | addition to the |
| | | | | | alternative promoter |
| | | | | | and exon 1, differential |
| | | | | | splicing of exons 71-74 |
| | | | | | and 78 produces at least |
| | | | | | five Dp140 isoforms. |
| | | | | | Of these, this transcript |
| | | | | | (Dp140b) lacks exon 78 |
| | | | | | and encodes a protein |
| | | | | | with a unique C- |
| | | | | | terminus. |
| Dystrophin | NM_004022.2 | 44 | NP_004013.1 | 45 | Transcript Variant: |
| Dp140ab | | | | | Dp140 transcripts use |
| isoform | | | | | exons 45-79, starting at |
| | | | | | a promoter/exon 1 |
| | | | | | located in intron 44. |
| | | | | | Dp140 transcripts have |
| | | | | | along (1 kb) 5′ UTR |
| | | | | | since translation is |
| | | | | | initiated in exon 51 |
| | | | | | (corresponding to aa |
| | | | | | 2461 of dystrophin). In |
| | | | | | addition to the |
| | | | | | alternative promoter |
| | | | | | and exon 1, differential |
| | | | | | splicing of exons 71-74 |
| | | | | | and 78 produces at least |
| | | | | | five Dp140 isoforms. |
| | | | | | Of these, this transcript |
| | | | | | (Dp140ab) lacks exons |
| | | | | | 71 and 78 and encodes |
| | | | | | a protein with a unique |
| | | | | | C-terminus. |
| Dystrophin | NM_004023.2 | 46 | NP_004014.1 | 47 | Transcript Variant: |
| Dp140bc | | | | | Dp140 transcripts use |
| isoform | | | | | exons 45-79, starting at |
| | | | | | a promoter/exon 1 |
| | | | | | located in intron 44. |
| | | | | | Dp140 transcripts have |
| | | | | | along (1 kb) 5′ UTR |
| | | | | | since translation is |
| | | | | | initiated in exon 51 |
| | | | | | (corresponding to aa |
| | | | | | 2461 of dystrophin). In |
| | | | | | addition to the |
| | | | | | alternative promoter |
| | | | | | and exon 1, differential |
| | | | | | splicing of exons 71-74 |
| | | | | | and 78 produces at least |
| | | | | | five Dp140 isoforms. |
| | | | | | Of these, this transcript |
| | | | | | (Dp140bc) lacks exons |
| | | | | | 71-74 and 78 and |
| | | | | | encodes a protein with |
| | | | | | a unique C-terminus. |
| Dystrophin | XM_006724469.3 | 48 | XP_006724532.1 | 49 | |
| isoform X2 | | | | | |
| Dystrophin | XM_011545467.1 | 50 | XP_011543769.1 | 51 | |
| isoform X5 | | | | | |
| Dystrophin | XM_006724473.2 | 52 | XP_006724536.1 | 53 | |
| isoform X6 | | | | | |
| Dystrophin | XM_006724475.2 | 54 | XP_006724538.1 | 55 | |
| isoform X8 | | | | | |
| Dystrophin | XM_017029328.1 | 56 | XP_016884817.1 | 57 | |
| isoform X4 | | | | | |
| Dystrophin | XM_006724468.2 | 58 | XP_006724531.1 | 59 | |
| isoform X1 | | | | | |
| Dystrophin | XM_017029331.1 | 60 | XP_016884820.1 | 61 | |
| isoform X13 | | | | | |
| Dystrophin | XM_006724470.3 | 62 | XP_006724533.1 | 63 | |
| isoform X3 | | | | | |
| Dystrophin | XM_006724474.3 | 64 | XP_006724537.1 | 65 | |
| isoform X7 | | | | | |
| Dystrophin | XM_011545468.2 | 66 | XP_011543770.1 | 67 | |
| isoform X9 | | | | | |
| Dystrophin | XM_017029330.1 | 68 | XP_016884819.1 | 69 | |
| isoform X11 | | | | | |
| Dystrophin | XM_017029329.1 | 70 | XP_016884818.1 | 71 | |
| isoform X10 | | | | | |
| Dystrophin | XM_011545469.1 | 72 | XP_011543771.1 | 73 | |
| isoform X12 | | | | | |

The murine dystrophin protein has the following amino acid sequence (Uniprot Accession No. P11531, SEQ. ID. NO. 74):

| | |
|---|---|
| 1 | MWWVDCYRDV KKTTKWNASK GKHDNSDDGK RDGTGKKKGS TRVHANNVNK ARVKNNVDVN |
| 61 | GSTDVDGNHK TGWNHWVKNV MKTMAGTNSK SWVRSTRNYV NVNTSSWSDG ANAHSHRDDW |
| 121 | NSVVSHSATR HANAKCGKDD VATTYDKKSM YTSVVSAVMR TSSKVTRHHH MHYSTVSAGY |
| 181 | TSSSKRKSYA TAAYVATSDS TSYSHARDKS DSSMTVNDSY TAVSWSADTR AGSNDVVKHA |
| 241 | HGMMDTSHGV GNVGSVGKGK SDAVMNNSRW CRVASMKSKH KVMDNKKDDW TKTRTKKMGD |
| 301 | DKCVHKVDVR VNSTHMVVVV DSSGDHATAA KVGDRWANCR WTDRWVDKWH TCSTWSKDAM |
| 361 | KNTSGKDNMM SSHKSTKDKK KTMKSSNDSA KNKSVTKMWM NARWDNTKKS SASAVTTTST |
| 421 | TTVMTVTMVT TRMVKHAKKR TVDSRKRDVD THSWTRSAVS SAVYRKGNSD KVNAARKAKR |
| 481 | KDASRSAAVM ANGVNASRAS NSRWTCSRVN WYTNTYNMTT TANKTSTTST AKSKCKDVNR |
| 541 | SAKSKKGGMD ADVATNHNHD GVRAKKTDTM RYTMSSRTWS SKSVYSVTYM RGKASSKNGN |
| 601 | YSDTVKMAKK ASCKYSGHWK KSSVSCKHMN KRKNHKTKWM AVDVKWAGDA KKKCRVGDTS |
| 661 | NSVNGGKKSA ASRTRNTWDH CRVYTRKAKA GDKTVSKDSM HWMTAYRDYK TDTAVMKRAK |
| 721 | AKTKVKTTVN SVAHASAAKK TTTNYWCTRN GKCKTVWACW HSYKANKWNV KKTMNVAGTV |
| 781 | SNMHHSNNRA TTDGGVMDNT NSRWRHAVRK KSSAKSHSDK AAYTDKVDAA MAKSDTSHSM |
| 841 | KKHNGKDANR VSDVAKKDVS MKRKANRSKM DVKMHATKSV VSSHCVNYKS SVKSVMVKTG |
| 901 | RVKKTNKDRV TAKHYNGAKV TRKKCKSRKM RKMNVTWAAT DTTKRSAVGM SNDSVAWGKA |
| 961 | TKKKAHKSVT GSKMVGKKTV DKSNSNWAVT SRVWNYKHMT DNTKWHADDS KKKKDKRKAM |
| 1021 | NDMRKVDSTR DAAKMANRGD HCRKVVSNRR AASHRKTGKA SKNSDKAGVN KDNKDMSDNG |
| 1081 | TVNRGDNRTD RKRKKTKHNA KDRSRRKKAS HWYYKRADDK CDKKASRDRK KDRKKKNAVR |
| 1141 | RAGSNGAAMA VTSKRWRSNA RRNAHTHTMV VTTDMDVSYV STYTSHASVD HNTCAKDDKS |
| 1201 | KNKDNSGRDH KKKTAASATS MKVKVAVAMD GKHRMYKRGR DRSVKWRHHY DMKVNWNVKK |
| 1261 | TNNWHAKYKW YKDGGRAVVR TNATGSSKTD VNKGSSRWHD CKARRKRKNV SRDNVWADNA |
| 1321 | TGDKVKARGK NTGGAVVSAR DKKKKTNWKV SRAKGVHKDR DHWSRNYNSA GDKVTVHGKA |
| 1381 | DVRSKGHYKK STVKRKDRSW AVNHRRTKDR AGSTTGASAS TVTVTSVVTK TVSKMSSVAA |
| 1441 | DNRAWTTDWS DRVKSRVMVG DDNMKKATDR RTAANKNKTS NARTTDRRWD VNRRNMKDST |
| 1501 | WAKAVGVRGK DSWKGHTVDA KKTTKAKDRR SVDVANDAKR DYSADDTRKV HMTNNTSWGN |
| 1561 | HKRVSAATHR DKSWTATTAN VDASRKKDSR GVRMKWDGTH TDYHNDNGKR SGSDARRDNM |
| 1621 | NKWSKKSNRS HASSDWKRHS VWKDDSRAGG DAVKNDHRAK RKTKVMSTTV RTGKYRRANV |
| 1681 | TRRKAVNAWD KNRSADWRKD ARAADDKRAV KGSWVGDDSD HKVKARGAKN VNRVNDAHTT |
| 1741 | GSYNSTDNTR WRVAVDRVRH AHRDGASHST SVGWRASNKV YYNHTTTCWD HKMTYSADNN |
| 1801 | VRSAYRTAMK RRKACDSSAA CDADHNKNDM DNCTTYDRHN NVNVCVDMCN WNVYDTGRTG |
| 1861 | RRVSKTGSCK AHDKYRYKVA SSTGCDRRGH DSRGVASGGS NSVRSCANNK AADWMRSMVW |
| 1921 | VHRVAAATAK HAKCNCKCGR YRSKHNYDCS CSGRVAKGHK MHYMVYCTTT SGDVRDAKVK |
| 1981 | NKRTKRYAKH RMGYVTVGDN MTVTNWVDSA ASSSHDDTHS RHYASRAMNS NGSYNDSSNS |
| 2041 | DDHHYCSNDS SRSASSRGRA DNRNAYDRKH HKGSSMMTSS RDAAAKRHKG RARMDHNKSH |
| 2101 | RRAAKVNGTT VSSSTSRSDS SMRVVGSTSS MGDSDTSTGV MNNSSSRGRN AGKMRDTM |

Dystrophin is an important component within muscle tissue that provides structural stability to the dystroglycan complex (DGC) of the cell membrane. While both sexes can carry the mutation, females are rarely affected with the skeletal muscle form of the disease.

Mutations vary in nature and frequency. Large genetic deletions are found in about 60-70% of cases, large duplications are found in about 10% of cases, and point mutants or other small changes account for about 15-30% of cases. One study examined some 7000 mutations and catalogued a total of 5,682 large mutations (80% of total mutations), of which 4,894 (86%) were deletions (1 exon or larger) and 784 (14%) were duplications (1 exon or larger). There were 1,445 small mutations (smaller than 1 exon, 20% of all mutations), of which 358 (25%) were small deletions and 132 (9%) small insertions, while 199 (14%) affected the splice sites. Point mutations totaled 756 (52% of small mutations) with 726 (50%) nonsense mutations and 30 (2%) missense mutations. Finally, 22 (0.3%) mid-intronic mutations were observed. In addition, mutations were identified within the database that would potentially benefit from novel genetic therapies for DMD including stop codon read-through therapies (10% of total mutations) and exon skipping therapy (80% of deletions and 55% of total mutations).

B. Symptoms

Symptoms usually appear in boys between the ages of 2 and 3 and may be visible in early infancy. Even though symptoms do not appear until early infancy, laboratory testing can identify children who carry the active mutation at birth. Progressive proximal muscle weakness of the legs and pelvis associated with loss of muscle mass is observed first. Eventually this weakness spreads to the arms, neck, and other areas. Early signs may include pseudohypertrophy (enlargement of calf and deltoid muscles), low endurance, and difficulties in standing unaided or inability to ascend staircases. As the condition progresses, muscle tissue experiences wasting and is eventually replaced by fat and fibrotic tissue (fibrosis). By age 10, braces may be required to aid in walking but most patients are wheelchair dependent by age 12. Later symptoms may include abnormal bone development that lead to skeletal deformities, including curvature of the spine. Due to progressive deterioration of muscle, loss of movement occurs, eventually leading to paralysis. Intellectual impairment may or may not be present but if present, does not progressively worsen as the child ages. The average life expectancy for males afflicted with DMD is around 25.

The main symptom of Duchenne muscular dystrophy, a progressive neuromuscular disorder, is muscle weakness associated with muscle wasting with the voluntary muscles being first affected, especially those of the hips, pelvic area, thighs, shoulders, and calves. Muscle weakness also occurs later, in the arms, neck, and other areas. Calves are often enlarged. Symptoms usually appear before age 6 and may appear in early infancy. Other physical symptoms are:
Awkward manner of walking, stepping, or running—(patients tend to walk on their forefeet, because of an increased calf muscle tone. Also, toe walking is a compensatory adaptation to knee extensor weakness.)
Frequent falls
Fatigue
Difficulty with motor skills (running, hopping, jumping)
Lumbar hyperlordosis, possibly leading to shortening of the hip-flexor muscles. This has an effect on overall posture and a manner of walking, stepping, or running.
Muscle contractures of Achilles tendon and hamstrings impair functionality because the muscle fibers shorten and fibrose in connective tissue
Progressive difficulty walking
Muscle fiber deformities
Pseudohypertrophy (enlarging) of tongue and calf muscles. The muscle tissue is eventually replaced by fat and connective tissue, hence the term pseudohypertrophy.
Higher risk of neurobehavioral disorders (e.g., ADHD), learning disorders (dyslexia), and non-progressive weaknesses in specific cognitive skills (in particular short-term verbal memory), which are believed to be the result of absent or dysfunctional dystrophin in the brain.
Eventual loss of ability to walk (usually by the age of 12)
Skeletal deformities (including scoliosis in some cases)
Trouble getting up from lying or sitting position
   The condition can often be observed clinically from the moment the patient takes his first steps, and the ability to walk usually completely disintegrates between the time the patient is 9 to 12 years of age. Most men affected with DMD become essentially “paralyzed from the neck down” by the age of 21. Muscle wasting begins in the legs and pelvis, then progresses to the muscles of the shoulders and neck, followed by loss of arm muscles and respiratory muscles. Calf muscle enlargement (pseudohypertrophy) is quite obvious. Cardiomyopathy particularly (dilated cardiomyopathy) is common, but the development of congestive heart failure or arrhythmia (irregular heartbeat) is only occasional.

A positive Gowers' sign reflects the more severe impairment of the lower extremities muscles. The child helps himself to get up with upper extremities: first by rising to stand on his arms and knees, and then “walking” his hands up his legs to stand upright. Affected children usually tire more easily and have less overall strength than their peers. Creatine kinase (CPK-MM) levels in the bloodstream are extremely high. An electromyography (EMG) shows that weakness is caused by destruction of muscle tissue rather than by damage to nerves. Genetic testing can reveal genetic errors in the Xp21 gene. A muscle biopsy (immunohistochemistry or immunoblotting) or genetic test (blood test) confirms the absence of dystrophin, although improvements in genetic testing often make this unnecessary.

Other symptoms include:
Abnormal heart muscle (cardiomyopathy)
Congestive heart failure or irregular heart rhythm (arrhythmia)
Deformities of the chest and back (scoliosis)
Enlarged muscles of the calves, buttocks, and shoulders (around age 4 or 5). These muscles are eventually replaced by fat and connective tissue (pseudohypertrophy).
Loss of muscle mass (atrophy)
Muscle contractures in the heels, legs
Muscle deformities
Respiratory disorders, including pneumonia and swallowing with food or fluid passing into the lungs (in late stages of the disease)

C. Causes

Duchenne muscular dystrophy (DMD) is caused by a mutation of the dystrophin gene at locus Xp21, located on the short arm of the X chromosome. Dystrophin is responsible for connecting the cytoskeleton of each muscle fiber to the underlying basal lamina (extracellular matrix), through a protein complex containing many subunits. The absence of dystrophin permits excess calcium to penetrate the sarcolemma (the cell membrane). Alterations in calcium and signaling pathways cause water to enter into the mitochondria, which then burst.

In skeletal muscle dystrophy, mitochondrial dysfunction gives rise to an amplification of stress-induced cytosolic calcium signals and an amplification of stress-induced reactive-oxygen species (ROS) production. In a complex cascading process that involves several pathways and is not clearly understood, increased oxidative stress within the cell damages the sarcolemma and eventually results in the death of the cell. Muscle fibers undergo necrosis and are ultimately replaced with adipose and connective tissue.

DMD is inherited in an X-linked recessive pattern. Females will typically be carriers for the disease while males will be affected. Typically, a female carrier will be unaware they carry a mutation until they have an affected son. The son of a carrier mother has a 50% chance of inheriting the defective gene from his mother. The daughter of a carrier mother has a 50% chance of being a carrier and a 50% chance of having two normal copies of the gene. In all cases, an unaffected father will either pass a normal Y to his son or a normal X to his daughter. Female carriers of an X-linked recessive condition, such as DMD, can show symptoms depending on their pattern of X-inactivation.

Exon deletions preceding exon 51 of the human DMD gene, which disrupt the open reading frame (ORF) by juxtaposing out of frame exons, represent the most common type of human DMD mutation. Skipping of exon 51 can, in principle, restore the DMD ORF in 13% of DMD patients with exon deletions.

Duchenne muscular dystrophy has an incidence of 1 in 5000 male infants. Mutations within the dystrophin gene can either be inherited or occur spontaneously during germline transmission. An exemplary but non-limiting mutation is deletion of exons 8 and 9 and a corresponding iPSC model is ΔEx8-9 iDMD and a mouse model is ΔEx8-9 DMD.

D. Diagnosis

Genetic counseling is advised for people with a family history of the disorder. Duchenne muscular dystrophy can be detected with about 95% accuracy by genetic studies performed during pregnancy.

DNA Test.

The muscle-specific isoform of the dystrophin gene is composed of 79 exons, and DNA testing and analysis can usually identify the specific type of mutation of the exon or exons that are affected. DNA testing confirms the diagnosis in most cases.

Muscle Biopsy.

If DNA testing fails to find the mutation, a muscle biopsy test may be performed. A small sample of muscle tissue is extracted (usually with a scalpel instead of a needle) and a dye is applied that reveals the presence of dystrophin. Complete absence of the protein indicates the condition.

Over the past several years DNA tests have been developed that detect more of the many mutations that cause the condition, and muscle biopsy is not required as often to confirm the presence of Duchenne's.

Prenatal Tests.

DMD is carried by an X-linked recessive gene. Males have only one X chromosome, so one copy of the mutated gene will cause DMD. Fathers cannot pass X-linked traits on to their sons, so the mutation is transmitted by the mother.

If the mother is a carrier, and therefore one of her two X chromosomes has a DMD mutation, there is a 50% chance that a female child will inherit that mutation as one of her two X chromosomes, and be a carrier. There is a 50% chance that a male child will inherit that mutation as his one X chromosome, and therefore have DMD.

Prenatal tests can tell whether an unborn child has the most common mutations. There are many mutations responsible for DMD, and some have not been identified, so genetic testing only works when family members with DMD have a mutation that has been identified.

Prior to invasive testing, determination of the fetal sex is important; while males are sometimes affected by this X-linked disease, female DMD is extremely rare. This can be achieved by ultrasound scan at 16 weeks or more recently by free fetal DNA testing. Chorion villus sampling (CVS) can be done at 11-14 weeks, and has a 1% risk of miscarriage. Amniocentesis can be done after 15 weeks, and has a 0.5% risk of miscarriage. Fetal blood sampling can be done at about 18 weeks. Another option in the case of unclear genetic test results is fetal muscle biopsy.

E. Treatment

There is no current cure for DMD, and an ongoing medical need has been recognized by regulatory authorities. Phase 1-2a trials with exon skipping treatment for certain mutations have halted decline and produced small clinical improvements in walking. Treatment is generally aimed at controlling the onset of symptoms to maximize the quality of life, and include the following:
Corticosteroids such as prednisolone and deflazacort increase energy and strength and defer severity of some symptoms.
Randomized control trials have shown that beta-2-agonists increase muscle strength but do not modify disease progression. Follow-up time for most RCTs on beta2-agonists is only around 12 months and hence results cannot be extrapolated beyond that time frame.
Mild, non-jarring physical activity such as swimming is encouraged. Inactivity (such as bed rest) can worsen the muscle disease.
Physical therapy is helpful to maintain muscle strength, flexibility, and function.
Orthopedic appliances (such as braces and wheelchairs) may improve mobility and the ability for self-care. Form-fitting removable leg braces that hold the ankle in place during sleep can defer the onset of contractures.
Appropriate respiratory support as the disease progresses is important.
   Comprehensive multi-disciplinary care standards/guidelines for DMD have been developed by the Centers for Disease Control and Prevention (CDC), and were published in two parts and are available at world-wide-web at treat-nmd.eu/dmd/care/diagnosis-management-DMD.

DMD generally progresses through five stages, as outlined in Bushby et al., Lancet Neurol., 9(1): 77-93 (2010) and Bushby et al., Lancet Neurol., 9(2): 177-198 (2010), incorporated by reference in their entireties. During the presymptomatic stage, patients typically show developmental delay, but no gait disturbance. During the early ambulatory stage, patients typically show the Gowers' sign, waddling gait, and toe walking. During the late ambulatory stage, patients typically exhibit an increasingly labored gait and begin to lose the ability to climb stairs and rise from the floor. During the early non-ambulatory stage, patients are typically able to self-propel for some time, are able to maintain posture, and may develop scoliosis. During the late non-ambulatory stage, upper limb function and postural maintenance is increasingly limited.

In some embodiments, treatment is initiated in the presymptomatic stage of the disease. In some embodiments, treatment is initiated in the early ambulatory stage. In some embodiments, treatment is initiated in the late ambulatory stage. In embodiments, treatment is initiated during the early non-ambulatory stage. In embodiments, treatment is initiated during the late non-ambulatory stage.

1. Physical Therapy

Physical therapists are concerned with enabling patients to reach their maximum physical potential. Their aim is to:
minimize the development of contractures and deformity by developing a program of stretches and exercises where appropriate
anticipate and minimize other secondary complications of a physical nature by recommending bracing and durable medical equipment
monitor respiratory function and advise on techniques to assist with breathing exercises and methods of clearing secretions

2. Respiration Assistance

Modem “volume ventilators/respirators,” which deliver an adjustable volume (amount) of air to the person with each breath, are valuable in the treatment of people with muscular dystrophy related respiratory problems. The ventilator may require an invasive endotracheal or tracheotomy tube through which air is directly delivered, but, for some people non-invasive delivery through a face mask or mouthpiece is sufficient. Positive airway pressure machines, particularly bi-level ones, are sometimes used in this latter way. The respiratory equipment may easily fit on a ventilator tray on the bottom or back of a power wheelchair with an external battery for portability.

Ventilator treatment may start in the mid to late teens when the respiratory muscles can begin to collapse. If the vital capacity has dropped below 40% of normal, a volume ventilator/respirator may be used during sleeping hours, a time when the person is most likely to be under ventilating (“hypoventilating”). Hypoventilation during sleep is determined by a thorough history of sleep disorder with an oximetry study and a capillary blood gas. A cough assist device can help with excess mucus in lungs by hyperinflation of the lungs with positive air pressure, then negative pressure to get the mucus up. If the vital capacity continues to decline to less than 30 percent of normal, a volume ventilator/respirator may also be needed during the day for more assistance. The person gradually will increase the amount of time using the ventilator/respirator during the day as needed.

F. Prognosis

Duchenne muscular dystrophy is a progressive disease which eventually affects all voluntary muscles and involves the heart and breathing muscles in later stages. The life expectancy is currently estimated to be around 25, but this varies from patient to patient. Recent advancements in medicine are extending the lives of those afflicted. The Muscular Dystrophy Campaign, which is a leading UK charity focusing on all muscle disease, states that “with high standards of medical care young men with Duchenne muscular dystrophy are often living well into their 30s.”

In rare cases, persons with DMD have been seen to survive into the forties or early fifties, with the use of proper positioning in wheelchairs and beds, ventilator support (via tracheostomy or mouthpiece), airway clearance, and heart medications, if required. Early planning of the required supports for later-life care has shown greater longevity in people living with DMD.

Curiously, in the mdx mouse model of Duchenne muscular dystrophy, the lack of dystrophin is associated with increased calcium levels and skeletal muscle myonecrosis. The intrinsic laryngeal muscles (ILM) are protected and do not undergo myonecrosis. ILM have a calcium regulation system profile suggestive of a better ability to handle calcium changes in comparison to other muscles, and this may provide a mechanistic insight for their unique pathophysiological properties. The ILM may facilitate the development of novel strategies for the prevention and treatment of muscle wasting in a variety of clinical scenarios.

### II. CRISPR SYSTEMS

A. CRISPRs

CRISPRs (clustered regularly interspaced short palindromic repeats) are DNA loci containing short repetitions of base sequences. Each repetition is followed by short segments of “spacer DNA” from previous exposures to a virus. CRISPRs are found in approximately 40% of sequenced eubacteria genomes and 90% of sequenced archaea. CRISPRs are often associated with Cas genes that code for proteins related to CRISPRs. The CRISPR/Cas system is a prokaryotic immune system that confers resistance to foreign genetic elements such as plasmids and phages and provides a form of acquired immunity. CRISPR spacers recognize and silence these exogenous genetic elements like RNAi in eukaryotic organisms.

CRISPR repeats range in size from 24 to 48 base pairs. They usually show some dyad symmetry, implying the formation of a secondary structure such as a hairpin, but are not truly palindromic. Repeats are separated by spacers of similar length. Some CRISPR spacer sequences exactly match sequences from plasmids and phages, although some spacers match the prokaryote's genome (self-targeting spacers). New spacers can be added rapidly in response to phage infection.

B. Cas Nucleases

CRISPR-associated (cas) genes are often associated with CRISPR repeat-spacer arrays. As of 2013, more than forty different Cas protein families had been described. Of these protein families, Cas1 appears to be ubiquitous among different CRISPR/Cas systems. Particular combinations of cas genes and repeat structures have been used to define 8 CRISPR subtypes (Ecoli, Ypest, Nmeni, Dvulg, Tneap, Hmari, Apem, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Exogenous DNA is apparently processed by proteins encoded by Cas genes into small elements (˜30 base pairs in length), which are then somehow inserted into the CRISPR locus near the leader sequence. RNAs from the CRISPR loci are constitutively expressed and are processed by Cas proteins to small RNAs composed of individual, exogenously-derived sequence elements with a flanking repeat sequence. The RNAs guide other Cas proteins to silence exogenous genetic elements at the RNA or DNA level. Evidence suggests functional diversity among CRISPR subtypes. The Cse (Cas subtype Ecoli) proteins (called CasA-E in E. coli) form a functional complex, Cascade, that processes CRISPR RNA transcripts into spacer-repeat units that Cascade retains. In other prokaryotes, Cas6 processes the CRISPR transcripts. Interestingly, CRISPR-based phage inactivation in E. coli requires Cascade and Cas3, but not Cas1 and Cas2. The Cmr (Cas RAMP module) proteins found in Pyrococcus furiosus and other prokaryotes form a functional complex with small CRISPR RNAs that recognizes and cleaves complementary target RNAs. RNA-guided CRISPR enzymes are classified as type V restriction enzymes.

Cas9 is a nuclease, an enzyme specialized for cutting DNA, with two active cutting sites, one for each strand of the double helix. The team demonstrated that they could disable one or both sites while preserving Cas9's ability to locate its target DNA. TracrRNA and spacer RNA can be combined into a “single-guide RNA” molecule that, mixed with Cas9, can find and cut the correct DNA targets, and such synthetic guide RNAs are able to be used for gene editing.

Cas9 proteins are highly enriched in pathogenic and commensal bacteria. CRISPR/Cas-mediated gene regulation may contribute to the regulation of endogenous bacterial genes, particularly during bacterial interaction with eukaryotic hosts. For example, Cas protein Cas9 of Francisella novicida uses a unique, small, CRISPR/Cas-associated RNA (scaRNA) to repress an endogenous transcript encoding a bacterial lipoprotein that is critical for F. novicida to dampen host response and promote virulence. One group showed that coinjection of Cas9 mRNA and sgRNAs into the germline (zygotes) generated nice with mutations. Delivery of Cas9 DNA sequences also is contemplated.

The systems CRISPR/Cas are separated into three classes. Class 1 uses several Cas proteins together with the CRISPR RNAs (crRNA) to build a functional endonuclease. Class 2 CRISPR systems use a single Cas protein with a crRNA. Cpf1 has been recently identified as a Class II, Type V CRISPR/Cas systems containing a 1,300 amino acid protein. See also U.S. Patent Publication 2014/0068797, which is incorporated by reference in its entirety.

In some embodiments, the compositions of the disclosure include a small version of a Cas9 from the bacterium Staphylococcus aureus (UniProt Accession No. J7RUA5). The small version of the Cas9 provides advantages over wild type or full length Cas9. In some embodiments the Cas9 is a spCas9 (AddGene).

C. Cpf1 Nucleases

Clustered Regularly Interspaced Short Palindromic Repeats from Prevotella and Francisella 1 or CRISPR/Cpf1 is a DNA-editing technology which shares some similarities with the CRISPR/Cas9 system. Cpf1 is an RNA-guided endonuclease of a class II CRISPR/Cas system. This acquired immune mechanism is found in Prevotella and Francisella bacteria. It prevents genetic damage from viruses. Cpf1 genes are associated with the CRISPR locus, coding for an endonuclease that use a guide RNA to find and cleave viral DNA. Cpf1 is a smaller and simpler endonuclease than Cas9, overcoming some of the CRISPR/Cas9 system limitations.

Cpf1 appears in many bacterial species. The ultimate Cpf1 endonuclease that was developed into a tool for genome editing was taken from one of the first 16 species known to harbor it.

In embodiments, the Cpf1 is a Cpf1 enzyme from Acidaminococcus (species BV3L6, UniProt Accession No. U2UMQ6; SEQ ID NO. 75), having the sequence set forth below:

| | |
|---|---|
| 1 | MTQFEGFTNL YQVSKTLRFE LIPQGKTLKH IQEQGFIEED KARNDHYKEL KPIIDRIYKT |
| 61 | YADQCLQLVQ LDWENLSAAI DSYRKEKTEE TRNALIEEQA TYRNAIHDYF IGRTDNLTDA |
| 121 | INKRHAEIYK GLFKAELFNG KVLKQLGTVT TTEHENALLR SFDKFTTYFS GFYENRKNVF |
| 181 | SAEDISTAIP HRIVQDNFPK FKENCHIFTR LITAVPSLRE HFENVKKAIG IFVSTSIEEV |
| 241 | FSFPFYNQLL TQTQIDLYNQ LLGGISREAG TEKIKGLNEV LNLAIQKNDE TAHIIASLPH |
| 301 | RFIPLFKQIL SDRNTLSFIL EEFKSDEEVI QSFCKYKTLL RNENVLETAE ALFNELNSID |
| 361 | LTHIFISHKK LETISSALCD HWDTLRNALY ERRISELTGK ITKSAKEKVQ RSLKHEDINL |
| 421 | QEIISAAGKE LSEAFKQKTS EILSHAHAAL DQPLPTTLKK QEEKEILKSQ LDSLLGLYHL |
| 481 | LDWFAVDESN EVDPEFSARL TGIKLEMEPS LSFYNKARNY ATKKPYSVEK FKLNFQMPTL |
| 541 | ASGWDVNKEK NNGAILFVKN GLYYLGIMPK QKGRYKALSF EPTEKTSEGF DKMYYDYFPD |
| 601 | AAKMIPKCST QLKAVTAHFQ THTTPILLSN NFIEPLEITK EIYDLNNPEK EPKKFQTAYA |
| 661 | KKTGDQKGYR EALCKWIDFT RDFLSKYTKT TSIDLSSLRP SSQYKDLGEY YAELNPLLYH |
| 721 | ISFQRIAEKE IMDAVETGKL YLFQIYNKDF AKGHHGKPNL HTLYWTGLFS PENLAKTSIK |
| 781 | LNGQAELFYR PKSRMKRMAH RLGEKMLNKK LKDQKTPIPD TLYQELYDYV NHRLSHDLSD |
| 841 | EARALLPNVI TKEVSHEIIK DRRFTSDKFF FHVPITLNYQ AANSPSKFNQ RVNAYLKEHP |
| 901 | ETPIIGIDRG ERNLIYITVI DSTGKILEQR SLNTIQQFDY QKKLDNREKE RVAARQAWSV |
| 961 | VGTIKDLKQG YLSQVIHEIV DLMIHYQAVV VLENLNFGFK SKRTGIAEKA VYQQFEKMLI |
| 1021 | DKLNCLVLKD YPAEKVGGVL NPYQLTDQFT SFAKMGTQSG FLFYVPAPYT SKIDPLTGFV |
| 1081 | DPFVWKTIKN HESRKHFLEG FDFLHYDVKT GDFILHFKMN RNLSFQRGLP GFMPAWDIVF |
| 1141 | EKNETQFDAK GTPFIAGKRI VPVIENHRFT GRYRDLYPAN ELIALLEEKG IVFRDGSNIL |
| 1201 | PKLLENDDSH AIDTMVALIR SVLQMRNSNA ATGEDYINSP VRDLNGVCFD SRFQNPEWPM |
| 1261 | DADANGAYHI ALKGQLLLNH LKESKDLKLQ NGISNQDWLA YIQELRN |

In some embodiments, the Cpf1 is a Cpf1 enzyme from Lachnospiraceae (species ND2006, UniProt Accession No. AOA182DWE3; SEQ ID NO. 76), having the sequence set forth below:

| | |
|---|---|
| 1 | AASKLEKFTN CYSLSKTLRF KAIPVGKTQE NIDNKRLLVE DEKRAEDYKG VKKLLDRYYL |
| 61 | SFINDVLHSI KLKNLNNYIS LFRKKTRTEK ENKELENLEI NLRKEIAKAF KGAAGYKSLF |
| 121 | KKDIIETILP EAADDKDEIA LVNSFNGFTT AFTGFFDNRE NMFSEEAKST SIAFRCINEN |
| 181 | LTRYISNMDI FEKVDAIFDK HEVQEIKEKI LNSDYDVEDF FEGEFFNFVL TQEGIDVYNA |
| 241 | IIGGFVTESG EKIKGLNEYI NLYNAKTKQA LPKFKPLYKQ VLSDRESLSF YGEGYTSDEE |
| 301 | VLEVFRNTLN KNSEIFSSIK KLEKLFKNFD EYSSAGIFVK NGPAISTISK DIFGEWNLIR |
| 361 | DKWNAEYDDI HLKKKAVVTE KYEDDRRKSF KKIGSFSLEQ LQEYADADLS VVEKLKEIII |
| 421 | QKVDEIYKVY GSSEKLFDAD FVLEKSLKKN DAVVAIMKDL LDSVKSFENY IKAFFGEGKE |
| 481 | TNRDESFYGD FVLAYDILLK VDHIYDAIRN YVTQKPYSKD KFKLYFQNPQ FMGGWDKDKE |
| 541 | TDYRATILRY GSKYYLAIMD KKYAKCLQKI DKDDVNGNYE KINYKLLPGP NKMLPKVFFS |
| 601 | KKWMAYYNPS EDIQKIYKNG TFKKGDMFNL NDCHKLIDFF KDSISRYPKW SNAYDFNFSE |
| 661 | TEKYKDIAGF YREVEEQGYK VSFESASKKE VDKLVEEGKL YMFQIYNKDF SDKSHGTPNL |
| 721 | HTMYFKLLFD ENNHGQIRLS GGAELFMRRA SLKKEELVVH PANSPIANKN PDNPKKTTTL |
| 781 | SYDVYKDKRF SEDQYELHIP IAINKCPKNI FKINTEVRVL LKHDDNPYVI GIDRGERNLL |
| 841 | YIVVVDGKGN IVEQYSLNEI INNFNGIRIK TDYHSLLDKK EKERFEARQN WTSIENIKEL |
| 901 | KAGYISQVVH KICELVEKYD AVIALEDLNS GFKNSRVKVE KQVYQKFEKM LIDKLNYMVD |
| 961 | KKSNPCATGG ALKGYQITNK FESFKSMSTQ NGFIFYIPAW LTSKIDPSTG FVNLLKTKYT |
| 1021 | SIADSKKFIS SFDRIMYVPE EDLFEFALDY KNFSRTDADY IKKWKLYSYG NRIRIFAAAK |
| 1081 | KNNVFAWEEV CLTSAYKELF NKYGINYQQG DIRALLCEQS DKAFYSSFMA LMSLMLQMRN |
| 1141 | SITGRTDVDF LISPVKNSDG IFYDSRNYEA QENAILPKNA DANGAYNIAR KVLWAIGQFK |
| 1201 | KAEDEKLDKV KIAISNKEWL EYAQTSVK |

In some embodiments, the Cpf1 is codon optimized for expression in mammalian cells. In some embodiments, the Cpf1 is codon optimized for expression in human cells or mouse cells.

The Cpf1 locus contains a mixed alpha/beta domain, a RuvC-I followed by a helical region, a RuvC-II and a zinc finger-like domain. The Cpf1 protein has a RuvC-like endonuclease domain that is similar to the RuvC domain of Cas9. Furthermore, Cpf1 does not have a HNH endonuclease domain, and the N-terminal of Cpf1 does not have the alpha-helical recognition lobe of Cas9.

Cpf1 CRISPR-Cas domain architecture shows that Cpf1 is functionally unique, being classified as Class 2, type V CRISPR system. The Cpf1 loci encode Cas1, Cas2 and Cas4 proteins more similar to types I and III than from type II systems. Database searches suggest the abundance of Cpf1-family proteins in many bacterial species.

Functional Cpf1 doesn't does not require a tracrRNA. Therefore, functional Cpf1 gRNAs of the disclosure may comprise or consist of a crRNA. This benefits genome editing because Cpf1 is not only a smaller than Cas9, but also it has a smaller sgRNA molecule (approximately half as many nucleotides as Cas9).

The Cpf1-gRNA (e.g., Cpf1-crRNA) complex cleaves target DNA or RNA by identification of a protospacer adjacent motif 5′-YTN-3′ (where “Y” is a pyrimidine and “N” is any nucleobase) or 5′-TTN-3′, in contrast to the G-rich PAM targeted by Cas9. After identification of PAM, Cpf1 introduces a sticky-end-like DNA double-stranded break of 4 or 5 nucleotides overhang.

The CRISPR/Cpf1 system comprises or consists of a Cpf1 enzyme and a guide RNA that finds and positions the complex at the correct spot on the double helix to cleave target DNA. In its native bacterial hosts, CRISPR/Cpf1 systems activity has three stages:
Adaptation, during which Cas1 and Cas2 proteins facilitate the adaptation of small fragments of DNA into the CRISPR array;
Formation of crRNAs: processing of pre-cr-RNAs producing of mature crRNAs to guide the Cas protein; and
Interference, in which the Cpf1 is bound to a crRNA to form a binary complex to identify and cleave a target DNA sequence.

This system has been modified to utilize non-naturally occurring crRNAs, which guide Cpf1 to a desired target sequence in a non-bacterial cell, such as a mammalian cell.

B. gRNA

As an RNA guided protein, Cas9 requires a short RNA to direct the recognition of DNA targets. Though Cas9 preferentially interrogates DNA sequences containing a PAM sequence NGG it can bind here without a protospacer target. However, the Cas9-gRNA complex requires a close match to the gRNA to create a double strand break. CRISPR sequences in bacteria are expressed in multiple RNAs and then processed to create guide strands for RNA. Because Eukaryotic systems lack some of the proteins required to process CRISPR RNAs the synthetic construct gRNA was created to combine the essential pieces of RNA for Cas9 targeting into a single RNA expressed with the RNA polymerase type III promoter U6. Synthetic gRNAs are slightly over 100 bp at the minimum length and contain a portion which targets the 20 protospacer nucleotides immediately preceding the PAM sequence NGG; gRNAs do not contain a PAM sequence.

In some embodiments, a gRNA targets a site within a wildtype dystrophin gene. In some embodiments, a gRNA targets a site within a mutant dystrophin gene. In some embodiments, a gRNA targets a dystrophin intron. In some embodiments, a gRNA targets a dystrophin exon. In some embodiments, a gRNA targets a site in a dystrophin exon that is expressed and is present in one or more of the dystrophin isoforms shown in Table 3. In some embodiments, a gRNA targets a site in a dystrophin exon that is within the ABD-1 domain of dystrophin. In embodiments, a gRNA targets a dystrophin splice site. In some embodiments, a gRNA targets a splice donor site on the dystrophin gene. In embodiments, a gRNA targets a splice acceptor site on the dystrophin gene.

In embodiments, more than one guide RNAs are used to edit a dystrophin gene. In some embodiments, 2, 3, 4, 5, 6, 7, 8, 9, or 10 guide RNAs are used to edit a dystrophin gene. In particular embodiments, two guide RNAs are used to edit a dystrophin gene.

In some embodiments, a first guide RNA targets a first genomic target sequence, and a second guide RNA targets a second genomic target sequence. In some embodiments, the first genomic target sequence and the second genomic target sequence may both be in an intronic region of the dsyrophin gene. In other embodiments, the first genomic target sequence and the second genomic target sequence may both be in an exonic region of the dsyrophin gene. In some embodiments, the first genomic target sequence is an intronic region of the dystropin gene, and the second genomic target sequence is in an exonic region of the dystrophin gene. In some embodiments, the genomic target sequence may be within intron 2, 3, 4, 5, 6, 7, 8, 9, 10, or 11 of dystrophin. In some embodiments, the genomic target sequence may be within exon 2, 3, 4, 5, 6, 7, 8, 9, 10, or 11 of dystrophin.

One or more guide RNAs may be used to edit the Actin Binding Domain-1 region of the dystrophin protein, encoded by exons 2-8 (amino acids 14-240 of SEQ ID NO: 74). For example, a guide RNA may target any one of exons 2-8, such as exon 2, 3, 4, 5, 6, 7 or 8. In some embodiments, a guide RNA may target any one of introns 2-9, such as intron 2, 3, 4, 5, 6, 7, 8, or 9. In some embodiments, a first guide RNA targets any one of exons 2-8, and a second guide RNA targets any one of exons 2-8. In some embodiments, a first guide RNA targets any one of introns 2-9, and a second guide RNA targets any one of introns 2-9. In some embodiments, a first guide RNA targets any one of introns 2-9, and a second guide RNA targets any one of exons 2-8. In some embodiments, a first guide RNA and a second guide RNA target the introns shown in Table 4, or the exons shown in Table 5, below. In these tables, an “x” indicates that the combination shown is contemplated by the instant disclosure.

TABLE 4
Combinations of Introns Targeted by First and Second gRNA

| | | Intron Targeted by First gRNA | | | | | | | |
|---|---|---|---|---|---|---|---|---|---|
| | | 2 | 3 | 4 | 5 | 6 | 7 | 8 | 9 |
| Intron | 2 | x | x | x | x | x | x | x | x |
| Targeted | 3 | x | x | x | x | x | x | x | x |
| by | 4 | x | x | x | x | x | x | x | x |
| Second | 5 | x | x | x | x | x | x | x | x |
| gRNA | 6 | x | x | x | x | x | x | x | x |
| | 7 | x | x | x | x | x | x | x | x |
| | 8 | x | x | x | x | x | x | x | x |
| | 9 | x | x | x | x | x | x | x | x |

TABLE 5
Combinations of Exons Targeted by First and Second gRNA

Exon Targeted by First gRNA

| | | 2 | 3 | 4 | 5 | 6 | 7 | 8 |
|---|---|---|---|---|---|---|---|---|
| Exon | 2 | x | x | x | x | x | x | x |
| Targeted | 3 | x | x | x | x | x | x | x |
| by | 4 | x | x | x | x | x | x | x |
| Second | 5 | x | x | x | x | x | x | x |
| gRNA | 6 | x | x | x | x | x | x | x |
| | 7 | x | x | x | x | x | x | x |
| | 8 | x | x | x | x | x | x | x |

In some embodiments, a first gRNA targets intron 2 and a second gRNA targets intron 7. In some embodiments, a first gRNA targets intron 5 and a second gRNA targets intron 7. In some embodiments, a first gRNA targets intron 2 and a second gRNA targets intron 9. In some embodiments, a first gRNA targets intron 7 and a second gRNA targets intron 9.

In some embodiments, a first genomic target sequence is located within intron 2 and a second genomic target sequence is located within intron 7. In some embodiments, a first genomic target sequence is located within intron 5 and a second genomic target sequence is located within intron 7. In some embodiments, a first genomic target sequence is located within intron 2 and a second genomic target sequence is located within intron 9. In some embodiments, a first genomic target sequence is located within intron 7 and a second genomic target sequence is located within intron 9.

In some embodiments, one or more gRNAs are used to delete one or more of exons 2-8. For example, a first gRNA may be targeted to a sequence in an intron 5′ to the one or more exons, and a second gRNA may be targeted to a sequence in an intron 3′ to the one or more exons. In the presence of a nuclease (e.g., Cas9 nuclease), the one or more gRNAs cause excision of the one or more exons. In some embodiments, exons 3-7 are deleted. In some embodiments, exons 6-7 are deleted.

Suitable gRNAs for use in various compositions and methods disclosed herein are provided as SEQ ID NOs. 6 to 10. (Table 1). In preferred embodiments, the gRNA is selected from any one of SEQ ID NO. 6 to SEQ ID NO. 10.

In some embodiments, gRNAs of the disclosure comprise a sequence that is complementary to a target sequence within a coding sequence or a non-coding sequence corresponding to the DMD gene, and, therefore, hybridize to the target sequence.

In some embodiments, gRNAs for Cpf1 comprise a single crRNA containing a direct repeat scaffold sequence followed by 24 nucleotides of guide sequence. In some embodiments, a “guide” sequence of the crRNA comprises a sequence of the gRNA that is complementary to a target sequence. In some embodiments, crRNA of the disclosure comprises a sequence of the gRNA that is not complementary to a target sequence. “Scaffold” sequences of the disclosure link the gRNA to the Cpf1 polypeptide. “Scaffold” sequences of the disclosure are not equivalent to a tracrRNA sequence of a gRNA-Cas9 construct.

E. Cas9 versus Cpf1

Cas9 requires two RNA molecules to cut DNA while Cpf1 needs one. The proteins also cut DNA at different places, offering researchers more options when selecting an editing site. Cas9 cuts both strands in a DNA molecule at the same position, leaving behind ‘blunt’ ends. Cpf1 leaves one strand longer than the other, creating ‘sticky’ ends that are easier to work with. Cpf1 appears to be more able to insert new sequences at the cut site, compared to Cas9. Although the CRISPR/Cas9 system can efficiently disable genes, it is challenging to insert genes or generate a knock-in. Cpf1 lacks tracrRNA, utilizes a T-rich PAM and cleaves DNA via a staggered DNA DSB.

In summary, important differences between Cpf1 and Cas9 systems are that Cpf1 recognizes different PAMs, enabling new targeting possibilities, creates 4-5 nt long sticky ends, instead of blunt ends produced by Cas9, enhancing the efficiency of genetic insertions and specificity during NHEJ or HDR, and cuts target DNA further away from PAM, further away from the Cas9 cutting site, enabling new possibilities for cleaving the DNA.

| Feature | Cas9 | Cpf1 |
|---|---|---|
| Structure | Two RNA required (Or 1 fusion | One RNA required |
| | transcript (crRNA + tracrRNA = | |
| | gRNA) | |
| Cutting | Blunt end cuts | Staggered end cuts |
| mechanism | | |
| Cutting site | Proximal to recognition site | Distal from recognition |
| | | site |
| Target sites | G-rich PAM | T-rich PAM |
| Cell type | Fast growing cells, including | Non-dividing cells, |
| | cancer cells | including nerve cells |

F. CRISPR/Cpf1-Mediated Gene Editing

The first step in editing the DMD gene using CRISPR/Cpf1 is to identify the genomic target sequence. The genomic target for the gRNAs of the disclosure can be any ˜24 nucleotide DNA sequence within the dystrophin gene, provided that the sequence is unique compared to the rest of the genome.

The next step in editing the DMD gene using CRISPR/Cpf1 is to identify all Protospacer Adjacent Motif (PAM) sequences within the genetic region to be targeted. Cpf1 utilizes a T-rich PAM sequence (TTTN, wherein N is any nucleotide). The target sequence must be immediately upstream of a PAM. Once all possible PAM sequences and putative target sites have been identified, the next step is to choose which site is likely to result in the most efficient on-target cleavage. The gRNA targeting sequence needs to match the target sequence, and the gRNA targeting sequence must not match additional sites within the genome. In preferred embodiments, the gRNA targeting sequence has perfect homology to the target with no homology elsewhere in the genome. In some embodiments, a given gRNA targeting sequence will have additional sites throughout the genome where partial homology exists. These sites are called “off-targets” and should be considered when designing a gRNA. In general, off-target sites are not cleaved as efficiently when mismatches occur near the PAM sequence, so gRNAs with no homology or those with mismatches close to the PAM sequence will have the highest specificity. In addition to “off-target activity”, factors that maximize cleavage of the desired target sequence (“on-target activity”) must be considered. It is known to those of skill in the art that two gRNA targeting sequences, each having 100% homology to the target DNA may not result in equivalent cleavage efficiency. In fact, cleavage efficiency may increase or decrease depending upon the specific nucleotides within the selected target sequence. Close examination of predicted on-target and off-target activity of each potential gRNA targeting sequence is necessary to design the best gRNA. Several gRNA design programs have been developed that are capable of locating potential PAM and target sequences and ranking the associated gRNAs based on their predicted on-target and off-target activity (e.g. CRISPRdirect, available at www.crispr.dbcls.jp).

The next step is to synthesize and clone desired gRNAs. Targeting oligos can be synthesized, annealed, and inserted into plasmids containing the gRNA scaffold using standard restriction-ligation cloning. However, the exact cloning strategy will depend on the gRNA vector that is chosen. The gRNAs for Cpf1 are notably simpler than the gRNAs for Cas9, and only consist of a single crRNA containing direct repeat scaffold sequence followed by ˜24 nucleotides of guide sequence. Cpf1 requires a minimum of 16 nucleotides of guide sequence to achieve detectable DNA cleavage, and a minimum of 18 nucleotides of guide sequence to achieve efficient DNA cleavage in vitro. In some embodiments, 20-24 nucleotides of guide sequence is used. The seed region of the Cpf1 gRNA is generally within the first 5 nucleotides on the 5′ end of the guide sequence. Cpf1 makes a staggered cut in the target genomic DNA. In AsCpf1 and LbCpf1, the cut occurs 19 bp after the PAM on the targeted (+) strand, and 23 bp on the other strand.

Each gRNA should then be validated in one or more target cell lines. For example, after the CRISPR and gRNA are delivered to the cell, the genomic target region may be amplified using PCR and sequenced according to methods known to those of skill in the art.

In some embodiments, gene editing may be performed in vitro or ex vivo. In some embodiments, cells are contacted in vitro or ex vivo with a Cpf1 and a gRNA that targets a dystrophin splice site. In some embodiments, the cells are contacted with one or more nucleic acids encoding the Cpf1 and the guide RNA. In some embodiments, the one or more nucleic acids are introduced into the cells using, for example, lipofection or electroporation. Gene editing may also be performed in zygotes. In embodiments, zygotes may be injected with one or more nucleic acids encoding Cpf1 and a gRNA that targets a dystrophin splice site. The zygotes may subsequently be injected into a host.

In embodiments, the Cpf1 is provided on a vector. In embodiments, the vector contains a Cpf1 sequence derived from a Lachnospiraceae bacterium. See, for example, Uniprot Accession No. AOA182DWE3; SEQ ID NO. 76. In embodiments, the vector contains a Cpf1 sequence derived from an Acidaminococcus bacterium. See, for example, Uniprot Accession No. U2UMQ6; SEQ ID NO. 75. In some embodiments, the Cpf1 sequence is codon optimized for expression in human cells or mouse cells. In some embodiments, the vector further contains a sequence encoding a fluorescent protein, such as GFP, which allows Cpf1-expressing cells to be sorted using fluorescence activated cell sorting (FACS). In some embodiments, the vector is a viral vector such as an adeno-associated viral vector.

In embodiments, the gRNA is provided on a vector. In some embodiments, the vector is a viral vector such as an adeno-associated viral vector. In embodiments, the Cpf1 and the guide RNA are provided on the same vector. In embodiments, the Cpf1 and the guide RNA are provided on different vectors.

In some embodiments, the cells are additionally contacted with a single-stranded DMD oligonucleotide to effect homology directed repair. In some embodiments, small INDELs restore the protein reading frame of dystrophin (“reframing” strategy). When the reframing strategy is used, the cells may be contacted with a single gRNA. In embodiments, a splice donor or splice acceptor site is disrupted, which results in exon skipping and restoration of the protein reading frame (“exon skipping” strategy). When the exon skipping strategy is used, the cells may be contacted with two or more gRNAs.

Efficiency of in vitro or ex vivo Cpf1-mediated DNA cleavage may be assessed using techniques known to those of skill in the art, such as the T7 E1 assay. Restoration of DMD expression may be confirmed using techniques known to those of skill in the art, such as RT-PCR, western blotting, and immunocytochemistry.

In some embodiments, in vitro or ex vivo gene editing is performed in a muscle or satellite cell. In some embodiments, gene editing is performed in iPSC or iCM cells. In embodiments, the iPSC cells are differentiated after gene editing. For example, the iPSC cells may be differentiated into a muscle cell or a satellite cell after editing. In embodiments, the iPSC cells are differentiated into cardiac muscle cells, skeletal muscle cells, or smooth muscle cells. In embodiments, the iPSC cells are differentiated into cardiomyocytes. iPSC cells may be induced to differentiate according to methods known to those of skill in the art.

In some embodiments, contacting the cell with the Cpf1 and the gRNA restores dystrophin expression. In embodiments, cells which have been edited in vitro or ex vivo, or cells derived therefrom, show levels of dystrophin protein that is comparable to wild type cells. In embodiments, the edited cells, or cells derived therefrom, express dystrophin at a level that is 50%, 60%, 70%, 80%, 90%, 95% or any percentage in between of wild type dystrophin expression levels. In embodiments, the cells which have been edited in vitro or ex vivo, or cells derived therefrom, have a mitochondrial number that is comparable to that of wild type cells. In embodiments the edited cells, or cells derived therefrom, have 50%, 60%, 70%, 80%, 90%, 95% or any percentage in between as many mitochondria as wild type cells. In embodiments, the edited cells, or cells derived therefrom, show an increase in oxygen consumption rate (OCR) compared to non-edited cells at baseline.

C. RNA Pol III and Pol III Promoters

In eukaryotes, RNA polymerase III (also called Pol III) transcribes DNA to synthesize ribosomal 5S rRNA, tRNA and other small RNAs. The genes transcribed by RNA Pol III fall in the category of “housekeeping” genes whose expression is required in all cell types and most environmental conditions. Therefore, the regulation of Pol III transcription is primarily tied to the regulation of cell growth and the cell cycle, thus requiring fewer regulatory proteins than RNA polymerase II. Under stress conditions however, the protein Maf1 represses Pol III activity.

In the process of transcription (by any polymerase) there are three main stages: (i) initiation, requiring construction of the RNA polymerase complex on the gene's promoter; (ii) elongation, the synthesis of the RNA transcript; and (iii) termination, the finishing of RNA transcription and disassembly of the RNA polymerase complex.

Promoters under the control of RNA Pol III include those for ribosomal 5S rRNA, tRNA and few other small RNAs such as U6 spliceosomal RNA, RNase P and RNase MRP RNA, 7SL RNA (the RNA component of the signal recognition particles), Vault RNAs, Y RNA, SINEs (short interspersed repetitive elements), 7SK RNA, two microRNAs, several small nucleolar RNAs and several few regulatory antisense RNAs

### III. NUCLEIC ACID DELIVERY

In some embodiments, one or more nucleic acids (e.g., an expression vector) are delivered to a cell. The cell may be a mammalian cell, for example a human cell, a mouse cell, or a dog cell. In some embodiments, the cell is an oocyte. In some embodiments, the cell is a non-human oocyte. In some embodiments the cell is a stem cell, such as an iPSC.

As discussed above, in certain embodiments, expression cassettes are employed to express a transcription factor product, either for subsequent purification and delivery to a cell/subject, or for use directly in a genetic-based delivery approach. Provided herein are expression vectors which contain one or more nucleic acids encoding Cpf1 and at least one DMD guide RNA that targets a dystrophin splice site. In some embodiments, a nucleic acid encoding Cpf1 and a nucleic acid encoding at least one guide RNA are provided on the same vector. In further embodiments, a nucleic acid encoding Cpf1 and a nucleic acid encoding least one guide RNA are provided on separate vectors.

Expression requires that appropriate signals be provided in the vectors, and include various regulatory elements such as enhancers/promoters from both viral and mammalian sources that drive expression of the genes of interest in cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

A. Regulatory Elements

Throughout this application, the term “expression cassette” is meant to include any type of genetic construct containing a nucleic acid coding for a gene product in which part or all of the nucleic acid encoding sequence is capable of being transcribed and translated, i.e., is under the control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene. An “expression vector” is meant to include expression cassettes comprised in a genetic construct that is capable of replication, and thus including one or more of origins of replication, transcription termination signals, poly-A regions, selectable markers, and multipurpose cloning sites.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

In some embodiments, the Cpf1 or Cas9 constructs of the disclosure are expressed by a muscle-cell specific promoter. This muscle-cell specific promoter may be constitutively active or may be an inducible promoter.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, viral promoters such as the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized. Further, selection of a promoter that is regulated in response to specific physiologic signals can permit inducible expression of the gene product.

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of promoters/enhancers and inducible promoters/enhancers that could be used in combination with the nucleic acid encoding a gene of interest in an expression construct. Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

The promoter and/or enhancer may be, for example, immunoglobulin light chain, immunoglobulin heavy chain, T-cell receptor, HLA DQ a and/or DQ β, β-interferon, interleukin-2, interleukin-2 receptor, MHC class II 5, MHC class II HLA-Dra, β-Actin, muscle creatine kinase (MCK), prealbumin (transthyretin), elastase I, metallothionein (MTII), collagenase, albumin, α-fetoprotein, t-globin, β-globin, c-fos, c-HA-ras, insulin, neural cell adhesion molecule (NCAM), α₁-antitrypain, H2B (TH2B) histone, mouse and/or type I collagen, glucose-regulated proteins (GRP94 and GRP78), rat growth hormone, human serum amyloid A (SAA), troponin I (TN I), platelet-derived growth factor (PDGF), duchenne muscular dystrophy, SV40, polyoma, retroviruses, papilloma virus, hepatitis B virus, human immunodeficiency virus, cytomegalovirus (CMV), and gibbon ape leukemia virus.

The promoter and/or enhancer may be, for example, immunoglobulin light chain, immunoglobulin heavy chain, T-cell receptor, HLA DQ a and/or DQ β, β-interferon, interleukin-2, interleukin-2 receptor, MHC class II 5, MHC class II HLA-Dra, β-Actin, muscle creatine kinase (MCK), prealbumin (transthyretin), elastase I, metallothionein (MTII), collagenase, albumin, α-fetoprotein, t-globin, β-globin, c-fos, c-HA-ras, insulin, neural cell adhesion molecule (NCAM), α₁-antitrypain, H2B (TH2B) histone, mouse and/or type I collagen, glucose-regulated proteins (GRP94 and GRP78), rat growth hormone, human serum amyloid A (SAA), troponin I (TN I), platelet-derived growth factor (PDGF), duchenne muscular dystrophy, SV40, polyoma, retroviruses, papilloma virus, hepatitis B virus, human immunodeficiency virus, cytomegalovirus (CMV), and gibbon ape leukemia virus.

In some embodiments, inducible elements may be used. In some embodiments, the inducible element is, for example, MTII, MMTV (mouse mammary tumor virus), β-interferon, adenovirus 5 E2, collagenase, stromelysin, SV40, murine MX gene, GRP78 gene, α-2-macroglobulin, vimentin, MHC class I gene H-2κb, HSP70, proliferin, tumor necrosis factor, and/or thyroid stimulating hormone a gene. In some embodiments, the inducer is phorbol ester (TFA), heavy metals, glucocorticoids, poly(rI)x, poly(rc), E1A, phorbol ester (TPA), interferon, Newcastle Disease Virus, A23187, IL-6, serum, interferon, SV40 large T antigen, PMA, and/or thyroid hormone. Any of the inducible elements described herein may be used with any of the inducers described herein.

Of particular interest are muscle specific promoters. These include the myosin light chain-2 promoter, the α-actin promoter, the troponin 1 promoter; the Na⁺/Ca²⁺ exchanger promoter, the dystrophin promoter, the α7 integrin promoter, the brain natriuretic peptide promoter and the αB-crystallin/small heat shock protein promoter, α-myosin heavy chain promoter and the ANF promoter. In some embodiments, the muscle specific promoter is the CK8 promoter. The CK8 promoter has the following sequence (SEQ ID NO. 77):

| | |
|---|---|
| 1 | CTAGACTAGC ATGCTGCCCA TGTAAGGAGG CAAGGCCTGG GGACACCCGA |
| | GATGCCTGGT |
| 61 | TATAATTAAC CCAGACATGT GGCTGCCCCC CCCCCCCCAA CACCTGCTGC |
| | CTCTAAAAAT |
| 121 | AACCCTGCAT GCCATGTTCC CGGCGAAGGG CCAGCTGTCC CCCGCCAGCT |
| | AGACTCAGCA |
| 181 | CTTAGTTTAG GAACCAGTGA GCAAGTCAGC CCTTGGGGCA GCCCATACAA |
| | GGCCATGGGG |
| 241 | CTGGGCAAGC TGCACGCCTG GGTCCGGGGT GGGCACGGTG CCCGGGCAAC |
| | GAGCTGAAAG |
| 301 | CTCATCTGCT CTCAGGGGCC CCTCCCTGGG GACAGCCCCT CCTGGCTAGT |
| | CACACCCTGT |
| 361 | AGGCTCCTCT ATATAACCCA GGGGCACAGG GGCTGCCCTC ATTCTACCAC |
| | CACCTCCACA |
| 421 | GCACAGACAG ACACTCAGGA GCCAGCCAGC |

In some embodiments, the muscle-cell cell specific promoter is a variant of the CK8 promoter, called CK8e. The CK8e promoter has the following sequence (SEQ ID NO. 78):

| | |
|---|---|
| 1 | TGCCCATGTA AGGAGGCAAG GCCTGGGGAC ACCCGAGATG CCTGGTTATA ATTAACCCAG |
| 61 | ACATGTGGCT GCCCCCCCCC CCCCAACACC TGCTGCCTCT AAAAATAACC CTGCATGCCA |
| 121 | TGTTCCCGGC GAAGGGCCAG CTGTCCCCCG CCAGCTAGAC TCAGCACTTA GTTTAGGAAC |
| 181 | CAGTGAGCAA GTCAGCCCTT GGGGCAGCCC ATACAAGGCC ATGGGGCTGG GCAAGCTGCA |
| 241 | CGCCTGGGTC CGGGGTGGGC ACGGTGCCCG GGCAACGAGC TGAAAGCTCA TCTGCTCTCA |
| 301 | GGGGCCCCTC CCTGGGGACA GCCCCTCCTG GCTAGTCACA CCCTGTAGGC TCCTCTATAT |
| 361 | AACCCAGGGG CACAGGGGCT GCCCTCATTC TACCACCACC TCCACAGCAC AGACAGACAC |
| 421 | TCAGGAGCCA GCCAGC |

Where a cDNA insert is employed, one will typically desire to include a polyadenylation signal to effect proper polyadenylation of the gene transcript. Any polyadenylation sequence may be employed such as human growth hormone and SV40 polyadenylation signals. Also contemplated as an element of the expression cassette is a terminator. These elements can serve to enhance message levels and to minimize read through from the cassette into other sequences.

B. 2A Protease

The inventor utilizes the 2A-like self-cleaving domain from the insect virus Thosea asigna (TaV 2A peptide; SEQ ID NO. 79; EGRGSLLTCGDVEENPGP). These 2A-like domains have been shown to function across Eukaryotes and cause cleavage of amino acids to occur co-translationally within the 2A-like peptide domain. Therefore, inclusion of TaV 2A peptide allows the expression of multiple proteins from a single mRNA transcript. Importantly, the domain of TaV when tested in eukaryotic systems has shown greater than 99% cleavage activity. Other acceptable 2A-like peptides include, but are not limited to, equine rhinitis A virus (ERAV) 2A peptide (SEQ ID NO. 80; QCTNYALLKLAGDVESNPGP), porcine teschovirus-1 (PTV1) 2A peptide (SEQ ID NO. 81; ATNFSLLKQAGDVEENPGP) and foot and mouth disease virus (FMDV) 2A peptide (SEQ ID NO. 82; PVKQLLNFDLLKLAGDVESNPGP) or modified versions thereof.

In some embodiments, the 2A peptide is used to express a reporter and a Cfp1 or a Cas9 simultaneously. The reporter may be, for example, GFP.

Other self-cleaving peptides that may be used include, but are not limited to nuclear inclusion protein a (Nia) protease, a P1 protease, a 3C protease, a L protease, a 3C-like protease, or modified versions thereof.

C. Delivery of Expression Vectors

There are a number of ways in which expression vectors may be introduced into cells. In certain embodiments, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells. These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals.

One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB. In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off. The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNAs issued from this promoter possess a 5′-tripartite leader (TPL) sequence which makes them preferred mRNAs for translation.

In one system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins. Since the E3 region is dispensable from the adenovirus genome, the current adenovirus vectors, with the help of 293 cells, carry foreign DNA in either the E1, the D3 or both regions. In nature, adenovirus can package approximately 105% of the wild-type genome, providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Methods for culturing 293 cells and propagating adenovirus are known to those of skill in the art. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

The adenoviruses of the disclosure are replication defective, or at least conditionally replication defective. The adenovirus may be of any of the 42 different known serotypes or subgroups A-F. Adenovirus type 5 of subgroup C is the preferred starting material in order to obtain the conditional replication-defective adenovirus vector for use as described herein.

As stated above, the typical vector of the disclosure is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical. The polynucleotide encoding the gene of interest may also be inserted in lieu of the deleted E3 region in E3 replacement vectors, or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹² plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus, demonstrating their safety and therapeutic potential as in vivo gene transfer vectors.

Adenovirus vectors have been used in eukaryotic gene expression and vaccine development. Animal studies suggested that recombinant adenovirus could be used for gene therapy. Studies in administering recombinant adenovirus to different tissues include trachea instillation, muscle injection, peripheral intravenous injections and stereotactic inoculation into the brain.

The retroviruses are a group of single-stranded RNA viruses characterized by an ability to convert their RNA to double-stranded DNA in infected cells by a process of reverse-transcription. The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome.

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. However, integration and stable expression require the division of host cells.

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses may be used, in which biotinylated antibodies against a retroviral envelope protein and against a specific cell receptor are used. The antibodies are coupled via the biotin components by using streptavidin. Using antibodies against major histocompatibility complex class I and class II antigens, it has been demonstrated the infection of a variety of human cells that bore those surface antigens with an ecotropic virus in vitro.

There are certain limitations to the use of retrovirus vectors. For example, retrovirus vectors usually integrate into random sites in the cell genome. This can lead to insertional mutagenesis through the interruption of host genes or through the insertion of viral regulatory sequences that can interfere with the function of flanking genes. Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination.

Other viral vectors may be employed as expression constructs. Vectors derived from viruses such as vaccinia virus, adeno-associated virus (AAV), and herpesviruses may be employed. They offer several attractive features for various mammalian cells.

In embodiments, the AAV vector is replication-defective or conditionally replication defective. In embodiments, the AAV vector is a recombinant AAV vector. In some embodiments, the AAV vector comprises a sequence isolated or derived from an AAV vector of serotype AAV1, AAV2, AAV3, AAV4, AAV5, AAV6, AAV7, AAV8, AAV9, AAV10, AAV11, or any combination thereof. In some embodiments, the AAV vector is not an AAV9 vector. In some embodiments, the AAV vector is an AAVRh.74 vector.

In some embodiments, a single viral vector is used to deliver a nucleic acid encoding Cpf1 or Cas9 and at least one gRNA to a cell. In some embodiments, Cpf1 or Cas9 is provided to a cell using a first viral vector and at least one gRNA is provided to the cell using a second viral vector. In order to effect expression of sense or antisense gene constructs, the expression construct must be delivered into a cell. The cell may be a muscle cell, a satellite cell, a mesangioblast, a bone marrow derived cell, a stromal cell or a mesenchymal stem cell. In embodiments, the cell is a cardiac muscle cell, a skeletal muscle cell, or a smooth muscle cell. In embodiments, the cell is a cell in the tibialis anterior, quadriceps, soleus, diaphragm or heart. In some embodiments, the cell is an induced pluripotent stem cell (iPSC) or inner cell mass cell (iCM). In further embodiments, the cell is a human iPSC or a human iCM. In some embodiments, human iPSCs or human iCMs of the disclosure may be derived from a cultured stem cell line, an adult stem cell, a placental stem cell, or from another source of adult or embryonic stem cells that does not require the destruction of a human embryo. Delivery to a cell may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present disclosure. These include calcium phosphate precipitation, DEAE-dextran, electroporation, direct microinjection, DNA-loaded liposomes, and lipofectamine-DNA complexes, cell sonication, gene bombardment using high velocity microprojectiles, and receptor-mediated transfection. Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. One group successfully injected polyomavirus DNA in the form of calcium phosphate precipitates into liver and spleen of adult and newborn mice demonstrating active viral replication and acute infection. Another group also demonstrated that direct intraperitoneal injection of calcium phosphate-precipitated plasmids results in expression of the transfected genes. DNA encoding a gene of interest may also be transferred in a similar manner in vivo and express the gene product.

In still another embodiment for transferring a naked DNA expression construct into cells may involve particle bombardment. This method depends on the ability to accelerate DNA-coated microprojectiles to a high velocity allowing them to pierce cell membranes and enter cells without killing them. Several devices for accelerating small particles have been developed. One such device relies on a high voltage discharge to generate an electrical current, which in turn provides the motive force. The microprojectiles used have consisted of biologically inert substances such as tungsten or gold beads.

In some embodiments, the expression construct is delivered directly to the liver, skin, and/or muscle tissue of a subject. This may require surgical exposure of the tissue or cells, to eliminate any intervening tissue between the gun and the target organ, i.e., ex vivo treatment. Again, DNA encoding a particular gene may be delivered via this method and still be incorporated by the present disclosure.

In a further embodiment, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Also contemplated are lipofectamine-DNA complexes.

Liposome-mediated nucleic acid delivery and expression of foreign DNA in vitro has been very successful. A reagent known as Lipofectamine 2000™ is widely used and commercially available.

In certain embodiments, the liposome may be complexed with a hemagglutinating virus (HVJ), to facilitate fusion with the cell membrane and promote cell entry of liposome-encapsulated DNA. In other embodiments, the liposome may be complexed or employed in conjunction with nuclear non-histone chromosomal proteins (HMG-1). In yet further embodiments, the liposome may be complexed or employed in conjunction with both HVJ and HMG-1. In that such expression constructs have been successfully employed in transfer and expression of nucleic acid in vitro and in vivo, then they are applicable for the present disclosure. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific.

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) and transferrin. A synthetic neoglycoprotein, which recognizes the same receptor as ASOR, has been used as a gene delivery vehicle and epidermal growth factor (EGF) has also been used to deliver genes to squamous carcinoma cells.

### IV. PHARMACEUTICAL COMPOSITIONS AND DELIVERY METHODS

For clinical applications, pharmaceutical compositions are prepared in a form appropriate for the intended application. Generally, this entails preparing compositions that are essentially free of pyrogens, as well as other impurities that could be harmful to humans or animals.

Appropriate salts and buffers are used to render drugs, proteins or delivery vectors stable and allow for uptake by target cells. Aqueous compositions of the present disclosure comprise an effective amount of the drug, vector or proteins, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Any conventional media or agent that is not incompatible with the active ingredients described herein, its use in therapeutic compositions may be used. Supplementary active ingredients also can be incorporated into the compositions, provided they do not inactivate the vectors or cells of the compositions.

In some embodiments, the active compositions of the present disclosure may include classic pharmaceutical preparations. Administration of these compositions according to the present disclosure may be via any common route so long as the target tissue is available via that route, but generally including systemic administration. This includes oral, nasal, or buccal.

Alternatively, administration may be by intradermal, subcutaneous, intramuscular, intraperitoneal or intravenous injection, or by direct injection into muscle tissue. Such compositions are normally administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

In some embodiments, the compositions of the present disclosure are formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like). Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like.)

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In some embodiments, the Cpf1 or Cas9 and gRNAs described herein may be delivered to the patient using adoptive cell transfer (ACT). In adoptive cell transfer, one or more expression constructs are provided ex vivo to cells which have originated from the patient (autologous) or from one or more individual(s) other than the patient (allogeneic). The cells are subsequently introduced or reintroduced into the patient. Thus, in some embodiments, one or more nucleic acids encoding Cpf1 or Cas9 and a guide RNA that targets a dystrophin splice site are provided to a cell ex vivo before the cell is introduced or reintroduced to a patient.

### V. DMD MYOEDITING

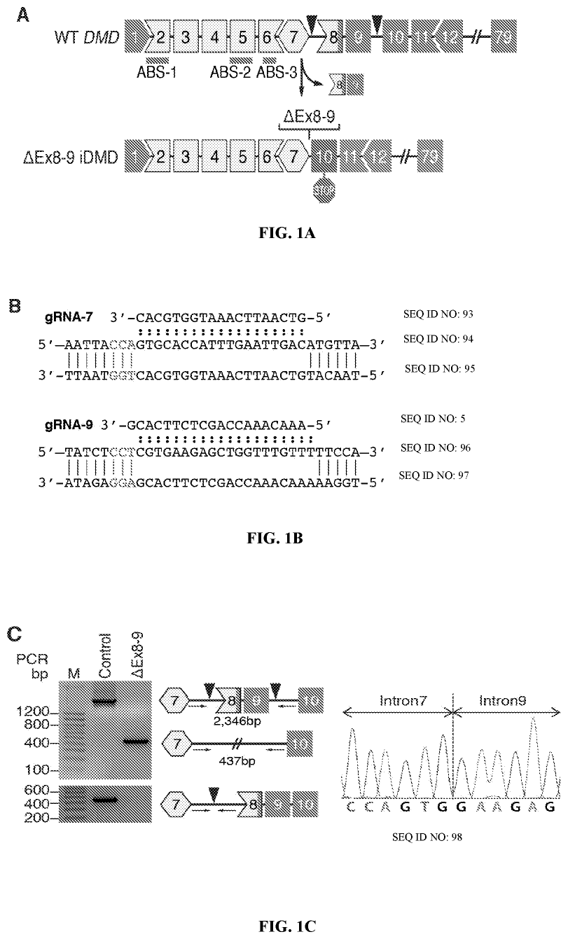

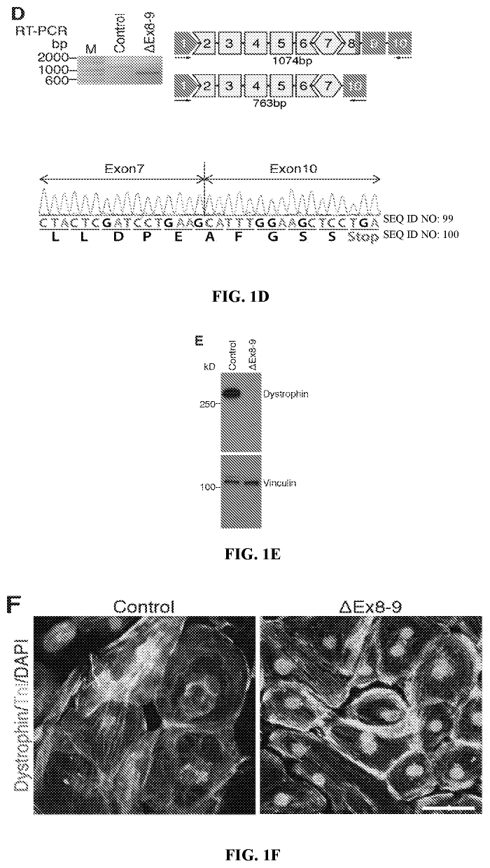

A dystrophin mutation that disrupts the actin-binding domain may be generated. The inventors generated a human iPSC line with a deletion of exons 8 and 9 in the DMD gene using CRISPR/Cas9-mediated editing to analyze the effect ABD-1 deletions on muscle function (FIG. 1A). Two single guide RNAs (gRNAs), targeting sequences in intron 7 and intron 9 of the DMD gene in the presence of Streptococcus pyogenes Cas9, were used to delete DMD exons 8-9 in healthy human iPSCs (FIG. 1B). This induced DMD mutant human cell line is referred to as ΔEx8-9 iDMD, and represents the common 5′-proximal hot spot mutations. Human iPSCs harboring DMD exons 8-9 deletions were picked by clonal selection and the mutation was confirmed by sequencing genomic DNA (FIG. 1C). The human ΔEx8-9 iDMD iPSC line has a 1,909 bp deletion, which generates a newly formed junction between intron 7 to intron 9 (FIG. 1C). Primers within intron 7 flanking the gRNA-7 targeting site were used to validate the deletion and as expected showed no PCR product with ΔEx8-9 iDMD compared to control iPSC with a 424 bp product (FIG. 1C). The ΔEx8-9 iDMD iPSCs were differentiated to cardiomyocytes and RT-PCR was performed using forward and reverse primers targeting exons 1 and 10, respectively (FIG. 1D). Sequencing of the RT-PCR products showed splicing of exon 7 to exon 10, which created a premature stop codon following the first 5 amino acids encoded by exon 10 (FIG. 1D). Loss of dystrophin protein was confirmed by Western blot analysis and immunocytochemistry in ΔEx8-9 iDMD iPSC-derived cardiomyocytes (FIGS. 1E-F).

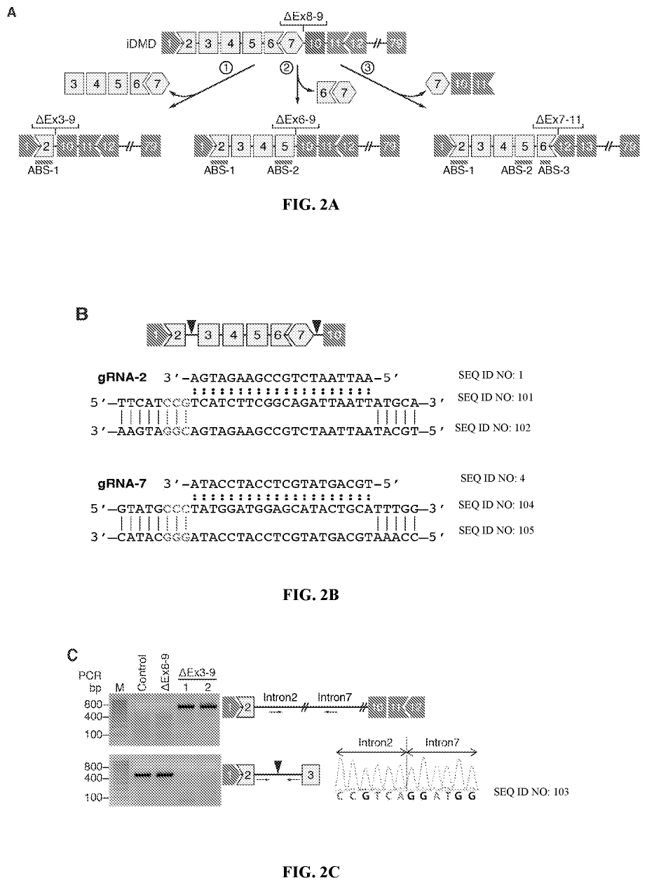

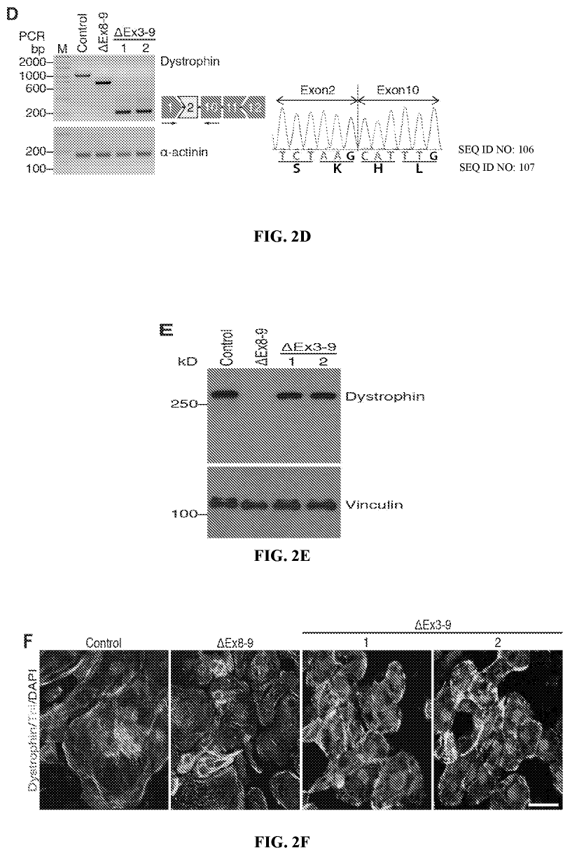

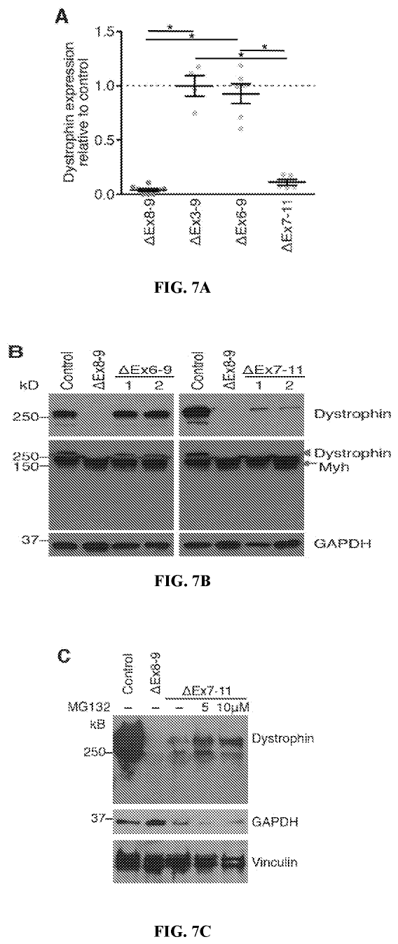

The ΔEx8-9 iDMD mutation may be corrected by exon deletion strategies. For example, three different CRISPR/Cas9-mediated strategies were used to correct the mutation in ΔEx8-9 iDMD iPSCs in order to restore the dystrophin open reading frame (FIG. 2A). These strategies generated dystrophin with different truncations in the ABD-1 domain, providing a means of analyzing the effect of ABD-1 deletions on muscle function. For each strategy, the inventors applied two gRNAs to delete multiple exons and analyzed two independent iPSC clones. Strategy #1 generated ΔEx3-9 iPSCs, by targeting intron 2 and intron 7 with gRNAs, and deleting exons 3 through 7 (FIG. 2B). Genomic sequencing of the genome edited region showed a hybrid junction of intron 2 to intron 7, confirming generation of ΔEx3-9 iPSCs (FIG. 2C). Following differentiation of ΔEx3-9 iPSCs to cardiomyocytes, RT-PCR was performed using primers within exon 1 and exon 10 (FIG. 2D). Sequencing of the RT-PCR product showed splicing of exon 2 to exon 10. Restoration of the DMD open reading frame generated a truncated dystrophin protein lacking the ABS-2 and ABS-3 regions. Dystrophin expression was confirmed by Western blot analysis and immunocytochemistry of ΔEx3-9 iPSC-derived cardiomyocytes (FIGS. 2E-F; FIG. 7A).

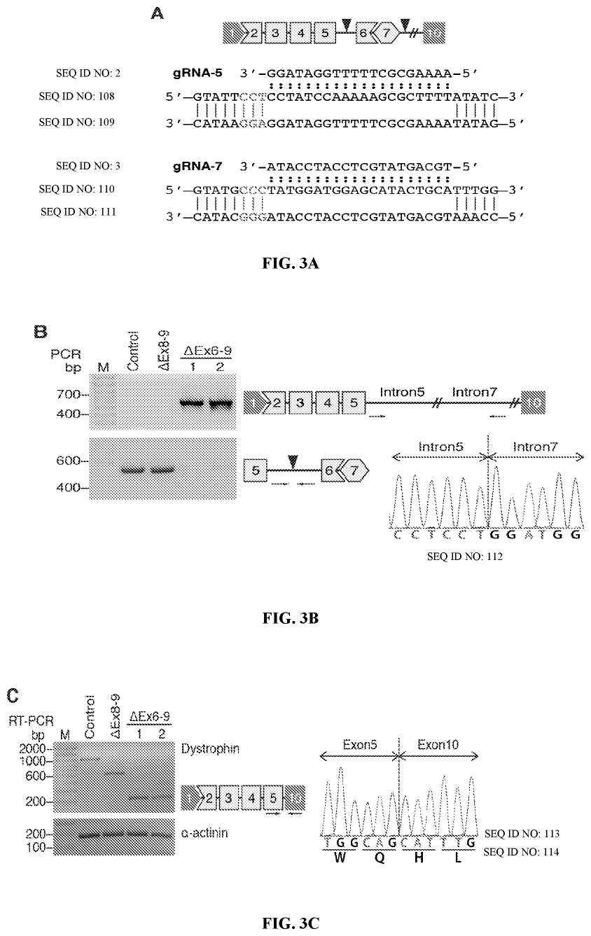

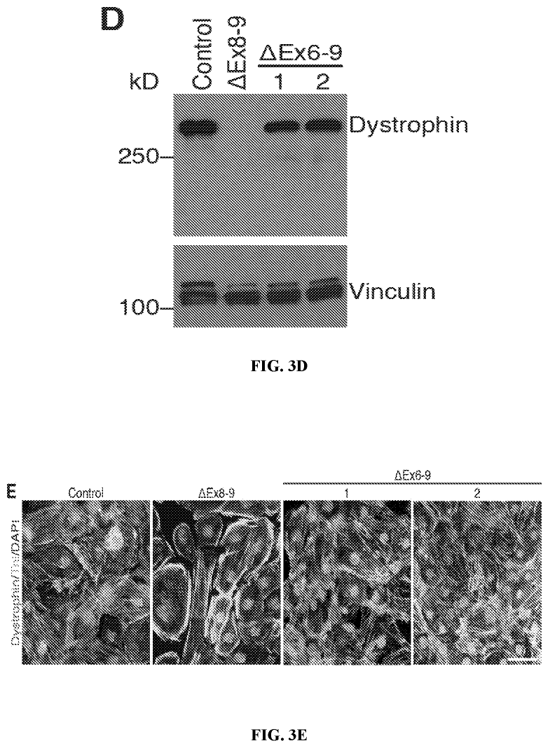

Strategy #2 generated ΔEx6-9 iPSCs, using gRNAs targeting intron 5 and intron 7 in order to delete exons 6 and 7 (FIG. 3A). This form of CRISPR/Cas9 editing resulted in deletion of exon 6 through exon 9, which was confirmed by sequencing of genomic PCR products (FIG. 3B). Following differentiation, RT-PCR using primers within exon 1 and exon 10 was performed on ΔEx6-9 iPSC-derived cardiomyocytes and sequencing of the RT-PCR product confirmed splicing of exon 5 to exon 10 (FIG. 3C). Restoration of dystrophin expression generated a truncated protein lacking the ABS-3 region. Dystrophin expression was confirmed by Western blot analysis and immunocytochemistry in ΔEx6-9 iPSC-derived cardiomyocytes (FIGS. 3D-E; FIGS. 7A-B).

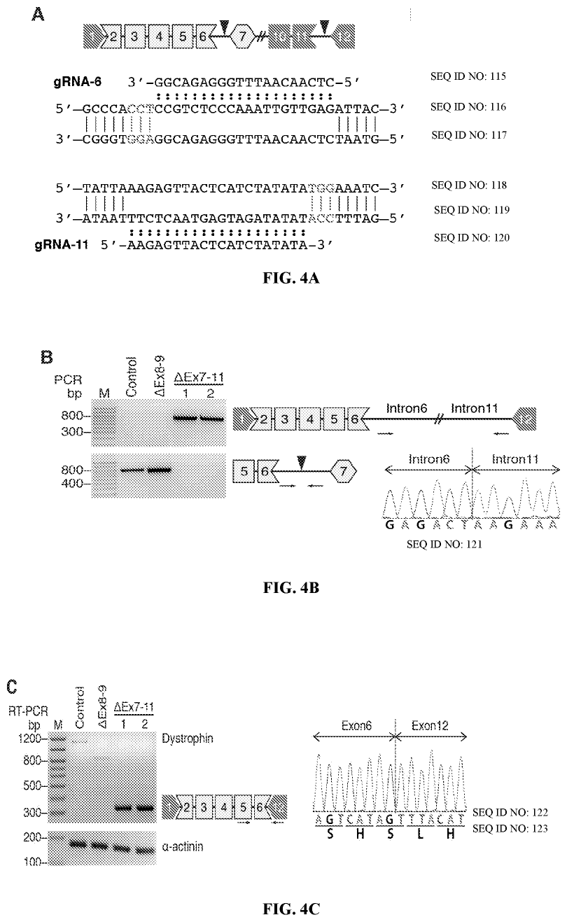

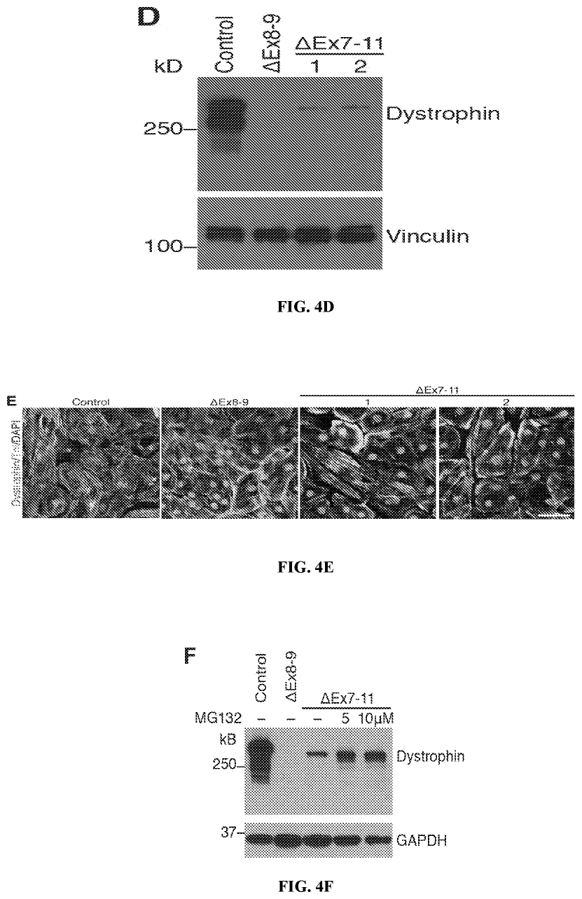

Strategy #3 generated ΔEx7-11 iPSCs, using gRNAs targeting introns 6 and 11 to delete exons 7 to 11 of ΔEx8-9 iDMD iPSCs (FIG. 4A). This genome editing strategy introduced the largest deletion of 164 kb in ΔEx8-9 iDMD iPSCs. Similar to the first two correction strategies, single cell-derived colonies were picked from the corrected iPSC pool and the genomic region was sequenced to confirm editing (FIG. 4B). The ΔEx7-11 iPSCs were differentiated to cardiomyocytes, and RT PCR was performed with primers within exon 5 and exon 12 to reveal splicing of exon 6 to exon 12, as seen by sequencing (FIG. 4C). Restoration of the DMD open reading frame of ΔEx7-11 iPSC-derived cardiomyocytes generated a truncated dystrophin protein lacking amino acids 177-444 but with all three ABS intact. Dystrophin expression was confirmed by Western blot analysis and immunocytochemistry (FIGS. 4D-E; FIGS. 7A-B). The inventors observed less dystrophin expression in ΔEx7-11 iPSC-derived cardiomyocytes compared to control iPSC-derived cardiomyocytes and the other corrected lines, ΔEx3-9 and ΔEx6-9. Previously, it was shown that missense mutations associated with the DMD phenotype caused steady-state decreases in dystrophin protein levels inversely proportional to the tertiary stability and directly caused by proteasomal degradation. Consistent with this report, the inventors observed increased dystrophin protein levels when ΔEx7-11 iPSC-derived cardiomyocytes were treated with the proteasome inhibitor MG-132, as measured by Western blot analysis (FIG. 4F; FIG. 7C). This suggests that deletion of DMD exons 7-11, which produces dystrophin lacking 177-444 amino acids, results in a truncated form of dystrophin that is degraded.

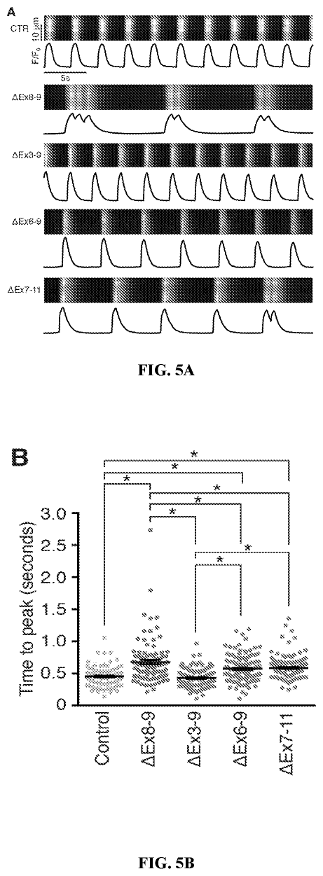

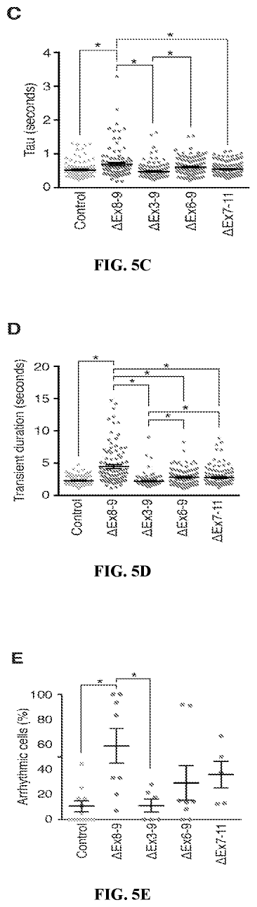

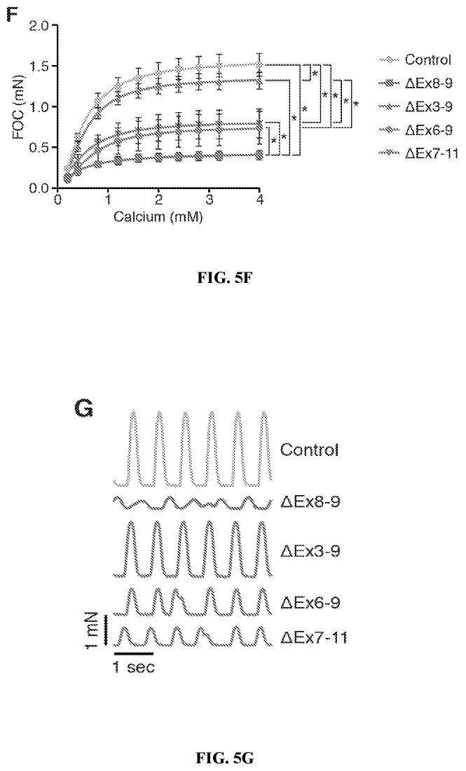

Functional restoration of iPSC-derived cardiomyocytes may be achieved by correction of ABD-1 mutations. To assess the consequences of ABD-1 mutations and the effect of correction by genome editing on dystrophin function in muscle, the inventors analyzed spontaneous Ca²⁺ activity in iPSC-derived cardiomyocytes (FIG. 5A). For each CRISPR/Cas9-edited correction, the inventors analyzed data sets from two corrected clones. As expected, the inventors observed that calcium release and reuptake parameters, including time to peak, Ca²⁺ decay rate, and transient duration were significantly higher in the ΔEx8-9 iDMD iPSC-derived cardiomyocytes compared to isogenic control cells (FIGS. 5B-D). Additionally, greater than 50% of the ΔEx8-9 iDMD iPSC-derived cardiomyocytes demonstrated abnormal Ca²⁺ activity, which represents an arrhythmogenic-like phenotype (FIG. 5E). In contrast, only 10.7% of the control iPSC-derived cardiomyocytes were desynchronized (FIG. 5E).

Corrected ΔEx3-9 iPSC-derived cardiomyocytes displayed normalized Ca²⁺ transient kinetics and decreased asynchronous activity similar to isogenic control cells, suggesting restoration of dystrophin function (FIGS. 5A-G). Cardiomyocytes derived from ΔEx6-9 iPSCs showed improved Ca²⁺ handling, although not to the level seen with ΔEx3-9 cells (FIGS. 5A-G). The Ca²⁺ decay rate and Ca²⁺ transient duration were shorter in ΔEx6-9 iPSC-derived cardiomyocytes (FIGS. 5B and 5D) and the number of arrhythmic cells was decreased to 29% compared to ΔEx8-9 iDMD cells (FIG. 5E). However, the parameters observed in the ΔEx6-9 iPSC-derived cardiomyocytes did not reach the levels of control iPSC-derived cardiomyocytes. The ΔEx7-11 iPSC-derived cardiomyocytes, similarly to ΔEx6-9 cells, showed improvement in Ca²⁺ release and uptake when compared to ΔEx8-9 iDMD cardiomyocytes (FIGS. 5B and 5C), however they did not reach control cell levels. iPSC-derived cardiomyocytes with ΔEx7-11 had the highest number of arrhythmic cells, up to 35.9%, among the corrected cell lines (FIG. 5E). Taken together, these findings indicate that deletion of exons 3-9 (ΔEx3-9) to correct DMD mutations within the 5′-proximal hot spot of the DMD gene produces a truncated dystrophin protein with restoration of function.

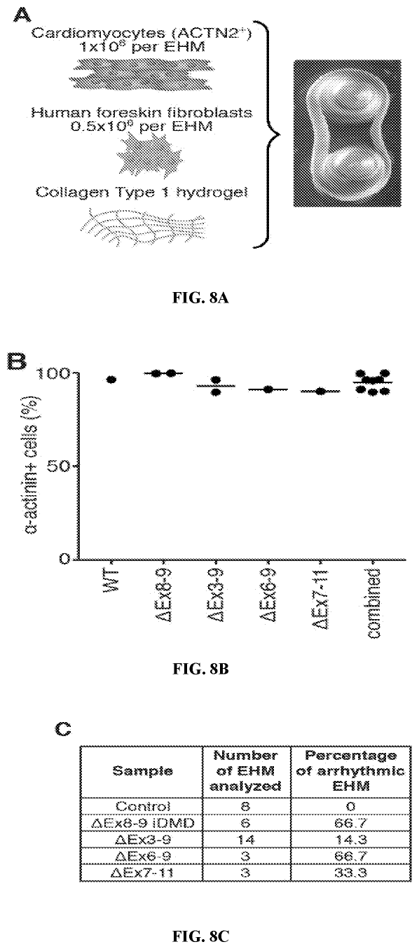

Further assessment of the functional properties of the iPSC-derived cardiomyocyte cell lines was performed by generating engineered heart muscle (EHM), which displays phenotypic properties of the postnatal myocardium. EHM was generated as shown in FIG. 8A using ˜95% pure iPSC-derived cardiomyocytes, as measured by α-actinin expression (FIG. 8B). Consistent with the calcium handling data, the inventors observed enhanced contractile performance in all corrected cell lines, with the most pronounced effects in ΔEx3-9 EHM (FIG. 5F). Moreover, the inventors observed an increased tendency for arrhythmic contractions in ΔEx8-9 iDMD-EHM and a reduction in ΔEx3-9 EHM (FIG. 8C).

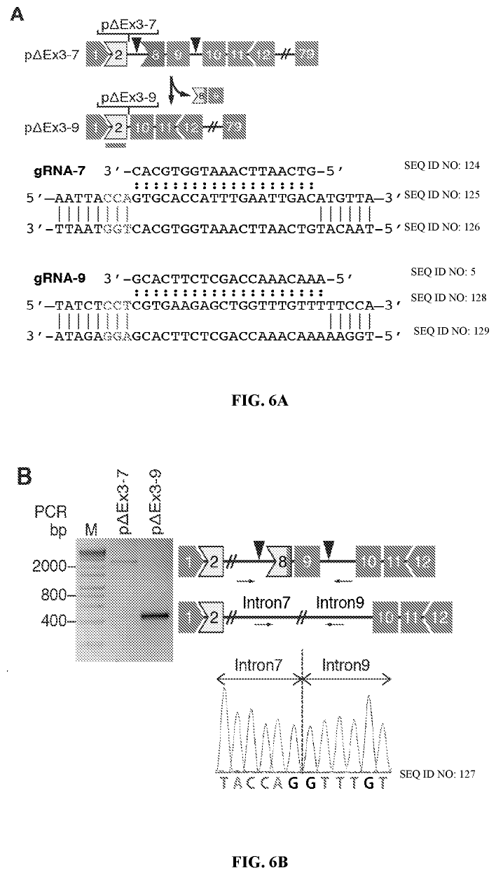

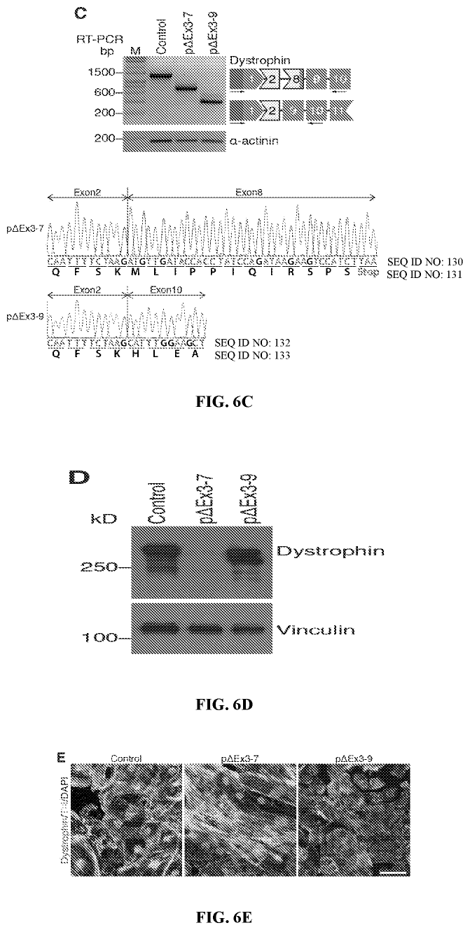

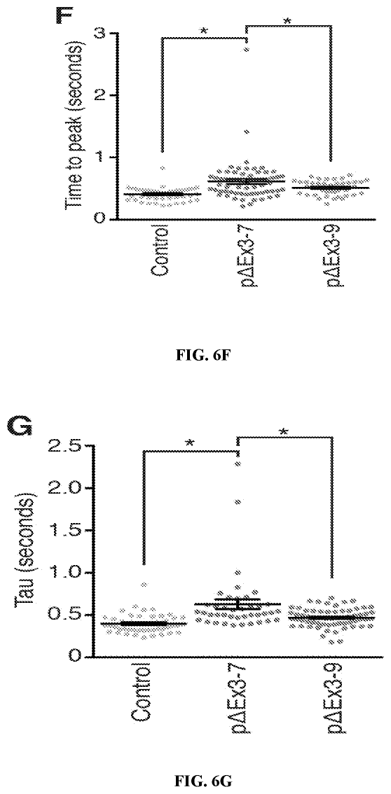

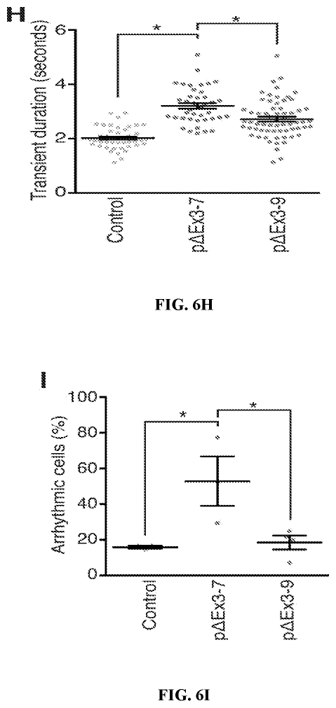

Correction of DMD patient-derived iPSCs may be achieved by deleting exons 3-9. An iPSC line, referred to as pΔEx3-7, was generated from a DMD patient (SC604A-MD) with a deletion of exons 3-7 in the DMD gene. pΔEx3-7 iPSCs are used as patient-in-a-dish model of DMD with a mutation in the ABD-1. To evaluate the effectiveness of exon 3-9 deletion in restoration of cardiomyocyte function, the inventors corrected pΔEx3-7 by deleting exons 8 and 9 to generate pΔEx3-9. Two gRNAs targeting introns 7 and 9 were used to excise exons 8-9, generating pΔEx3-9 iPSCs (FIG. 6A). Sequencing of genomic DNA PCR products confirmed the hybrid junction of intron 7 to intron 9 in a single cell-derived iPSC colony (FIG. 6B). Both pΔEx3-7 and pΔEx3-9 iPSC lines were differentiated into cardiomyocytes and RT-PCR was performed with primers targeting the 5′-UTR and exon 10. In pΔEx3-7 iPSC-derived cardiomyocytes, the junction of exon 2 to exon 8 was seen by sequencing of the RT-PCR product (FIG. 6C). In corrected pΔEx3-9 iPSC-derived cardiomyocytes, the junction of exon 2 to exon 10 was demonstrated by sequencing the RT-PCR product (FIG. 6C). Western blot analysis showed a low level of dystrophin expression in DMD pΔEx3-7 iPSC-derived cardiomyocytes (FIG. 6D). This is consistent with previous reports showing low levels of dystrophin protein expression in patients lacking exons 3-7 due to re-initiation of translation at an in-frame start codon in exon 8 (Fletcher et al., 2012; Winnard et al., 1995). Corrected pΔEx3-9 iPSC-derived cardiomyocytes showed dystrophin expression similar to normal control cardiomyocytes (FIG. 6D). These findings were confirmed by immunocytochemistry (FIG. 6E).

Spontaneous Ca²⁺ activity was assessed in these patient-derived cells as a measure of iPSC-derived cardiomyocyte functionality (FIGS. 6F-I). In pΔEx3-7 DMD iPSC-derived cardiomyocytes, Ca²⁺ time to peak and time to half decay were elevated (FIGS. 6F-G). This caused an overall slower Ca²⁺ transient (FIG. 6H) and elevated the number of arrhythmic cells, up to 53% (FIG. 6I). Corrected pΔEx3-9 iPSC-derived cardiomyocytes had significantly improved time to peak and faster Ca²⁺ decay (FIGS. 6F-G). Although these parameters of the corrected pΔEx3-9 iPSC-derived cardiomyocytes did not reach normal control levels, the number of arrhythmic cells was 18.5%, similar to control iPSC-derived cardiomyocytes (FIG. 6I). These findings show improved iPSC-derived cardiomyocyte function after genomic editing to excise exons 3-9 of the DMD gene in DMD patient-derived iPSCs.

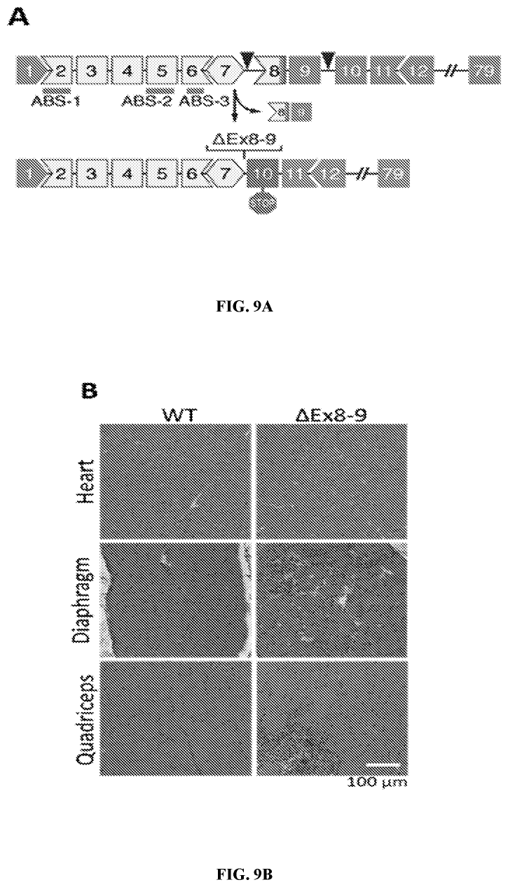

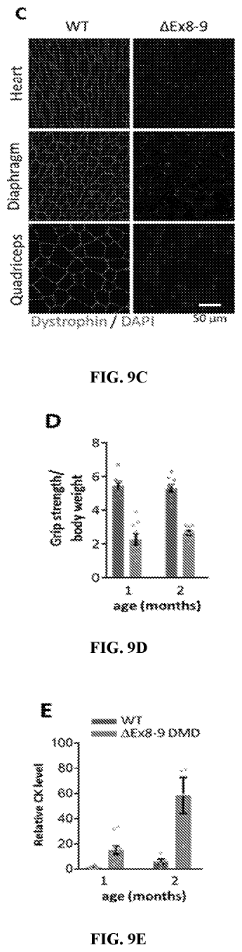

ΔEx8-9 DMD mouse models may be used to recapitulate the muscle dystrophy phenotype. To investigate CRISPR/Cas9-mediated exon skipping and reframing in vivo, a mimic of the human ABD-1 region mutations was generated in a mouse model by deleting exons 8 and 9, using CRISPR/Cas9 system directed by two single guide RNAs (sgRNA) (FIG. 9A and Table 2). The inventors designed and validated sgRNAs targeting introns, flanking 5′ end and 3′ ends of Dmd exons 8 and 9 respectively. C57BL/6 zygotes were co-injected with in vitro transcribed Cas9 mRNA and in vitro transcribed sgRNAs, and then re-implanted into pseudo-pregnant females.

The deletion of Dmd exons 8-9 was confirmed by DNA genotyping. Mice lacking exons 8-9 showed pronounced dystrophic muscle changes in 1 and 2 month-old mice (FIG. 9B). The deletion of these exons placed the dystrophin gene out of frame leading to the absence of dystrophin protein in skeletal muscle and heart (FIG. 9C). The grip strength of the ΔEx8-9 DMD was significantly weaker than that in wild-type mice (FIG. 9D). Serum analysis of the ΔEx8-9 DMD mice shows a significant increase of creatine kinase (CK) level, which is a sign of muscle damage (FIG. 9E). Taken together, dystrophin protein expression, muscle histology, muscle strength and serum creatine kinase level validated the dystrophic phenotype of the ΔEx8-9 DMD mouse model.

Dystrophin protein serves as a muscular shock absorber by providing a structural link between the cytoskeleton and the extracellular matrix to maintain muscle integrity. Essential regions of the dystrophin protein are located at both ends of the protein; the amino-terminus contains ABD-1, the actin binding domain, and the carboxy-terminus includes binding sites for components of the dystrophin associated protein complex, such as sarcoglycans and dystroglycans. Genomic mutations in the DMD gene encoding non-essential regions have been excised by exon skipping and deletion to restore functional, albeit truncated, dystrophin protein. Here, the inventors use CRISPR/Cas9-mediated editing to selectively delete exons 3-9 of the DMD gene encoding part of the essential ABD-1 region. With precision genomic editing, they can modify the essential amino-terminal region and restore dystrophin expression and cardiomyocyte function.

To provide an isogenic control iPSC line for these studies, the inventors generated an iDMD model (ΔEx8-9) by deleting DMD exons 8 and 9 in a healthy donor-derived iPSC line. They confirmed ΔEx8-9 iDMD iPSCs as a model of DMD by showing that cardiomyocytes derived from these cells do not express dystrophin protein and display arrhythmias attributable to impaired Ca²⁺ cycling. The lack of dystrophin results in the loss of linkage between the extracellular matrix and the actin cytoskeleton, destabilizing membrane proteins and resulting in generation of reactive oxygen species and excessive Ca²⁺ entry. Consistent with these findings, it was shown that cardiomyocytes differentiated from DMD iPSCs have slower Ca²⁺ cycling. Additionally, others have reported cellular abnormalities such as elevated levels of resting Ca²⁺, mitochondrial damage and apoptosis in DMD-derived iPSC cardiomyocytes. Complementary to the phenotype of DMD iPSC-derived cardiomyocytes, DMD patients develop cardiomyopathy, fibrosis and cardiac arrhythmias. Taken together, this corroborates the inventors' findings that deletion of exons 8 and 9 of the DMD gene in normal iPSCs generates an iDMD model, and that cardiomyocytes derived from ΔEx8-9 iDMD serve as a model of “DMD in a dish”.

The approach reported here is to use CRISPR/Cas9-mediated genomic editing to restore the open reading of the DMD gene and measure the functional outcome of truncated dystrophin in muscle. One of the issues of using CRISPR/Cas9 editing is the occurrence of unintended, off-target, genomic cleavage. Reassuringly, previous reports showed that CRISPR/Cas9-modified human iPSC clones do not exhibit elevated off-target mutation rates, supporting the use of edited iPSC clones for disease modeling. Nevertheless, the inventors recognize that Cas9 nuclease poses a potential safety concern for clinical applicability and acknowledge that whole-genome sequencing should be performed in future in vivo studies to identify potential off-target changes in the genome.

Mutations in the ABD-1 region are genotypically and phenotypically variable in patients. In some cases, out-of-frame ABD-1 mutations display a BMD phenotype, most likely due to the presence of an alternative translation initiation site in exons 6 or exon 8. Interestingly, it was shown that deletion of exon 2 activates an alternative translation initiation site in exon 6 to generate truncated dystrophin. However, duplication of exon 2, which results in a stop codon within the duplicated exon 2, does not activate an alternative translation initiation site. To correct the duplication of exon 2, a U7 small nuclear RNA was used against exon 2 to skip one or both copies of exon 2, generating either full length or truncated dystrophin. Another approach used to correct exon 2 duplication was to use CRISPR/Cas9-mediated editing with one gRNA directed against a duplicated intronic region, resulting in precise deletion of one of the duplicated exons and restoration the DMD gene. Patients with deletion of exons 3-7 display variable phenotypes, depending on whether the alternative translation initiation site in exon 8 is used. However, in general, patients with ABD-1 mutations display a severe BMD phenotype or are diagnosed as DMD. Studies using micro- and mini-dystrophins as therapeutic approaches to treat DMD showed that the truncated dystrophins remain functional if they lack portions of the central rod domain. However, restoration of muscle function by expression of micro-dystrophin in the mdx mouse model of DMD must include an intact ABD-1 region.

Correcting the ABD-1 mutation in DMD is not exclusively based on restoring the open reading frame. Functional assessment of the truncated dystrophin isoforms that are modified in the ABD-1 region is essential. There are three actin-binding sites (ABSs) localized within the ABD-1 region: ABS1 (amino acids 18-27 encoded by exon 2); ABS2 (amino acids 88-116 encoded by exon 5); and ABS3 (amino acids 131-147 encoded by exon 6). The three approaches to correct ΔEx8-9 iDMD iPSCs by CRISPR/Cas9-mediated editing produced different modifications to the dystrophin ABD-1 region, such that corrected ΔEx3-9 retained the ABS1; corrected ΔEx6-9 retained both ABS1 and ABS2; and corrected ΔEx7-11 retained all three actin binding sites. Unexpectedly, the ΔEx7-11 correction, which retained most of the ABD-1 region, created the least stable protein, showing minimal restoration of function. The ΔEx7-11 iPSC-derived cardiomyocytes expressed low levels of dystrophin protein compared to isogenic control cells and the corrected ΔEx3-9 and ΔEx6-9 lines. The decrease in dystrophin expression in the ΔEx7-11 iPSC-derived cardiomyocytes resulted in slower Ca²⁺ cycling and higher propensity for arrhythmias. In eDystrophin (edystrophin.genouest.org), a database dedicated to human dystrophin variants produced by in-frame DMD gene mutations, a patient with the ΔEx7-11 DMD deletion was annotated as DMD. It has also been reported that a single missense point mutation in exon 11 causes DMD protein mis-folding and dysfunction, resulting in a BMD phenotype. Therefore, the inventors conclude that although the deletion of exons 7-11 maintains the open reading frame of the DMD gene, the absence of amino acids 178-444 causes protein mis-folding and subsequent degradation. Indeed, by inhibiting proteasome activity, truncated dystrophin protein increased in ΔEx7-11 iPSC-derived cardiomyocytes. Furthermore, it was reported that when recreated in vitro, point mutations in the ABD-1 region that cause a human DMD phenotype are associated with decreased dystrophin protein levels due to proteasomal degradation.

Correction of DMD ΔEx8-9 mutation by deleting exons 6-9 retained ABS1 and ABS2 and improved Ca²⁺ activity in cardiomyocytes, but did not fully restore function to control levels. Shorter time to peak in these cells indicates faster Ca²⁺ release from the intracellular stores. However, slower Ca²⁺ decay in these cells compared to control cells suggests that their Ca²⁺ re-uptake system was not completely restored, resulting in arrhythmias. A similar strategy was applied to correct the ABD-1 mutation in a golden retriever dog model of DMD, which harbors a mutation in the exon 7 splice site. Some functional improvement was seen when exons 6-8 were deleted, resulting in splicing of exon 5 to 9 or to exon 10 due to occurrence of a cryptic splice site in exon 9.

Among the three genome-editing correction strategies the inventors tested, ΔEx3-9 which generated the truncated dystrophin lacking amino acid residues 32-320, was the most effective in restoring functionality of iPSC-derived cardiomyocytes. The inventors used CRISPR/Cas9 to create ΔEx3-9 using ΔEx8-9 iDMD or DMD patient-derived pΔEx3-7 iPSCs, and they restored iPSC-derived cardiomyocyte function in both ΔEx3-9 corrected cell lines. ΔEx3-9 produces truncated dystrophin that retains ABS1. Consistent with the inventors' findings, it was shown that overexpressing the dystrophin isoform lacking ABS2 and ABS3, prevents severe dystrophy in mdx mice. In addition, by surveying different databases, one group identified 15 patients with an exon 3-9 deletion, 11 of whom were asymptomatic or diagnosed as BMD. In fact, one of these patients is a competitive badminton player and was asymptomatic until he was diagnosed with BMD at age 67. His dystrophin level was recorded using two different dystrophin antibodies, as 47% of the control level using a C-terminus antibody and 62% of the control level using a rod domain antibody. Another of these patients expressed only 15% of normal dystrophin levels and showed a slight decrease in cardiac function at age 21 with no obvious skeletal muscle involvement. Similarly, in mouse models, at least 20% of full-length or central rod domain-deleted dystrophin expression is required to rescue the mdx phenotype. In human mutations involving the 5′-proximal region of the DMD gene, 30% or more of dystrophin expression is required to prevent diagnosis of muscular dystrophy in X-linked dilated cardiomyopathy patients. Taken together, these reports show in both mice and humans that deletion of exons 3-9 of the DMD gene produces a stable, albeit truncated dystrophin protein capable of maintaining function even when expressed at low levels.

The functional relevance of the ΔEx3-9 mutation and its potential therapeutic superiority compared to ΔEx6-9 and ΔEx7-11 was further underscored by phenotypic screening in EHM. All three models resulted in improved contractile performance compared to the iDMD model, however, ΔEx3-9-EHM showed the most enhanced contractile force and lowest propensity for arrhythmic contractions. This suggests deletion of exons 3-9 of the DMD gene as a practical candidate for exon-deletion which could be applicable to ˜7% of DMD patients' mutations. The other two correction strategies that deleted exons 6-9 or 7-11, each could potentially correct ˜2% of DMD patients' mutations based on the UMD-DMD database. Although shown as a possible gene therapy approach for correcting DMD, the inventors are aware that deletion of exons 3-9 of the DMD gene generates a large genome deletion up to ˜150 kb. To this point, large genome deletions by CRISPR/Cas9-mediated editing of up to 725 kb of the DMD gene have been previously reported to delete exons 45-55 in human cells in vitro, and in a humanized DMD mouse model in vivo.

To test efficiency of CRISPR/Cas9-mediated deletion of exons 3-9 of the DMD gene by two gRNAs and the function of the truncated dystrophin in vivo, the inventors generated the ΔEx8-9 DMD mouse model, which presents dystrophic phenotype. This model could be used to test multiple exon skipping as well as exons reframing approaches to correct dystrophin ABD-1 mutations.

In summary, the inventors' human iPSC results provide evidence that deletion of exons 3-9 in the DMD gene restores muscle function and is applicable for using CRIPSR/Cas9 genomic editing to correct ABD-1 mutations that were previously not addressed. The functional properties of these dystrophin ABD-1 mutations will dictate the most efficacious CRISPR/Cas9 strategy for possible genomic editing to correct DMD mutations within the proximal hot spot of the DMD gene allowing effective restoration of dystrophin function.

### VI. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Study Approval.

All experimental procedures involving animals in this study were reviewed and approved by the University of Texas Southwestern Medical Center's Institutional Animal Care and Use Committee.

Human iPSC Maintenance, Nucleofection and Differentiation.

Human healthy donor induced pluripotent stem cells were reprogrammed from peripheral blood mononuclear cells with CytoTune-iPS Sendai Reprogramming Kit (catalog # A16518; Thermo Fisher Scientific). Human Muscular Dystrophy iPS Cell Line (catalog #SC604A-MD; Systems Biosciences Inc.) were referred to as pΔEx3-7. Human iPSCs were cultured in mTeSR™1 media (catalog #05850; STEMCELL Technologies) and passaged approximately every 3-4 days (1:12-1:18 split ratio). One hour before nucleofection, iPSCs were treated with 10 μM ROCK inhibitor, Y-27632 (catalog #S1049, Selleckchem) and dissociated into single cells using Accutase (catalog #A6964 Innovative Cell Technologies, Inc.). 1×10⁶ iPS cells were mixed with 6 μg total of pSpCas9(BB)-2A-GFP (PX458) from Feng Zhang (MIT, Cambridge, Mass.) Addgene plasmid #48138 (Ran et al., 2013) plasmid and nucleofected using the P3 Primary Cell 4D-Nucleofector X kit (catalog #V4XP-3024; Lonza) according to manufacturer's protocol. After nucleofection, iPSCs were cultured in mTeSR™1 media supplemented with 10 μM ROCK inhibitor and 100 μg/ml Primocin (InvivoGen), and the next day the media was switched to fresh mTeSR™1. Three days post-nucleofection, GFP(+) and GFP(−) cells were sorted by FACS and subjected to genotyping by PCR. Single clones derived from GFP(+) iPSCs were picked, genotyped and sequenced. iPSCs were induced to differentiate into cardiomyocytes, using previously described protocol with modifications. When cells reached ˜80% confluency the medium was changed to CDM3 every other day until day 10. On day 0-2, medium was supplemented with 4 μM of CHIR99021 (catalog #S2924; Selleckchem). On day 2-4 medium was supplemented with 2 μM WNT-C59 (catalog #S7037; Selleckchem). On day 10 after differentiation initiation, media was changed to RPMI 1640 medium, no glucose (catalog #11879020; Thermo Fisher Scientific) supplemented with B27-supplement (catalog #17504044; Thermo Fisher Scientific) for 10 days. On day 20 media was switched to cells maintaining media, RPMI 1640 medium, (catalog #11875093; Thermo Fisher Scientific) supplemented with B27-supplement. Cardiomyocytes were used for experiments on day 30-40 after initiation of differentiation.

Genomic DNA Isolation.

Genomic DNA of human iPSCs was isolated using DirectPCR (Cell) (catalog #302-C; Viagen) according to manufacturer's recommendations.

RT-PCR.

RNA from iPSC-derived cardiomyocytes was isolated using TRIzol (catalog #15596026; ThermoFisher Scientific), and extracted with Direct-zol™ RNA MiniPrep kit according to manufacturer's protocol. cDNA was synthesized using iScript™ gDNA Clear cDNA Synthesis Kit (catalog #1725034; Bio-Rad Laboratories) according to manufacturer's instructions. 2 g of cDNA per reaction was used for RT-PCR reaction using Taq polymerase for 40 cycles. Primer pairs used for human DMD RT-PCR were as follows:

(SEQ ID NO: 83)
Exon 1 forward: 5′-CTTTGGTGGGAAGAAGTAGAGGACTG-3′
(SEQ ID NO: 84)
5′-UTR forward: 5′-CTTTCCCCCTACAGGACTCAG-3′
(SEQ ID NO: 85)
Exon 5 forward: 5′-TTGGAAGTACTGACATCGTAGATGGA-3′
(SEQ ID NO: 86)
Exon 10 reverse: 5′-CTCAGCAGAAAGAAGCCACGATAATA-3′
(SEQ ID NO: 87)
Exon 12 reverse: 5′-TGTTAGCCAGTCATTCAACTCTTTCA-3′

PCR products for exon 1 forward exon 10 reverse were 1073 bps in ctrl iPSC-CM 762 bps in ΔEx8-9 iDMD iPSC-CM and 206 bps in ΔEx3-9 iPSC-CM. PCR products for exon 5 forward exon 10 reverse were 797 bps in ctrl iPSC-CM 486 bps in ΔEx8-9 iDMD iPSC-CM and 194 bps in ΔEx6-9 iPSC-CM. PCR products for exon 5 forward exon 12 reverse were 1115 bps in ctrl iPSC-CM 804 bps in ΔEx8-9 iDMD iPSC-CM and 314 bps in ΔEx7-11 iPSC-CM. PCR products for 5′-UTR forward exon 10 reverse were 1345 bps in ctrl iPSC-CM 748 bps in pΔEx3-7 iPSC-CM and 544 bps in pΔEx3-9 iPSC-CM. PCR products were run on 1.5% agarose gel, excised and sequenced. Primer pair used for human α-actinin control is:

(SEQ ID NO: 88)
ACTN2 forward: 5′-CAACTTCAACACGCTGCAGACCAA-3′
(SEQ ID NO: 89)
ACTN2 reverse: 5′-AAGCGCTCCAGTCTTCGAATCTCA-3′

Dystrophin Western Blot Analysis.

iPSC-CMs were collected in cold PBS on ice, centrifuged and lysed with RIPA lysis buffer: 150 mM NaCl, 0.5% sodium deoxycholate, 0.1% SDS, 5% glycerol, 50 mM Tris-HCl pH 7.4, 1% Nonidet P40. Samples were needle-homogenized, big clumps were removed by centrifugation and supernatant was incubated at 95° C. 10 min in the presence of Laemmli sample buffer. 10 μg of protein per sample was separated on Mini-PROTEAN TGX 4-20% pre-cast gel (BioRad) for 3 hr at 100V. Proteins were transferred to PVDF membrane at 100V for 1.5 hr. Membranes were probed with mouse anti-dystrophin antibody MANDYS8 (1:1000) (catalog #D8168; Sigma) and with mouse anti-vinculin antibody, hVIN-1 (1:1000) (catalog #V9131; Sigma). For analysis of DEx7-11 iPSC-derived cardiomyocytes treated with proteasome inhibitor MG132, membranes were cut and the upper portion was probed with mouse MANDYS8 anti-dystrophin antibody (1:1000) (catalog # D8168; Sigma) and the lower portion with mouse anti-GAPDH antibody, (1:1000) (catalog # MAB374; 6C5, EMD Milipore). Goat anti-mouse HRP-conjugated secondary antibodies were used (1:10,000) (catalog #172-1011; Bio-Rad).

Dystrophin and Troponin-I Immunocytochemistry.

iPSC-derived cardiomyocytes (1×10⁵) seeded on 12 mm coverslips coated with poly-D-lysine and Matrigel (catalog #354248; Corning,) were removed from culture media and fixed in cold acetone (10 minutes, −20° C.). Following fixation, coverslips were equilibrated in phosphate-buffered saline, pH 7.3 (PBS) and then blocked for one-hour with serum cocktail (2% normal horse serum/2% normal donkey serum/0.2% bovine serum albumin (BSA/PBS)). Excess blocking cocktail was removed and dystrophin/troponin primary antibody cocktail, mouse anti-dystrophin (1:800) (catalog #D8168; MANDYS8, Sigma-Aldrich) and rabbit anti-troponin-I (1:200) (catalog #sc-15368; H170, Santa Cruz Biotechnology) in 0.2% BSA/PBS was applied without an intervening wash. Following overnight incubation at 4° C., unbound primary antibodies were removed with PBS washes, and coverslips were probed for one-hour with secondary antibodies, biotinylated horse anti-Mouse IgG (1:200) (Vector Laboratories) and fluorescein-conjugated donkey anti-rabbit IgG (1:50) (Jackson Immunoresearch) diluted in 0.2% BSA/PBS. Unbound secondary antibodies were removed with PBS washes and final dystrophin labeling done with 10-minute incubation of rhodamine-avidin-DCS (1:60) (Vector Laboratories) diluted in PBS. Unbound rhodamine was removed with PBS washes, nuclei were counterstained with 2 μg/ml Hoechst 33342 (Molecular Probes, Eugene, Oreg.), and coverslips were inverted onto slides overlaying Vectashield fluorescent mounting media (Vector Laboratories).

Calcium Imaging.

iPSC-derived cardiomyocytes were dissociated with TryplE (catalog #12605036; Thermo Fisher Scientific) and single cells were plated at low density on Matrigel coated 35 mm glass-bottom dishes and Ca²⁺ handling was measured 3-4 days after plating. To monitor changes in cytoplasmic Ca²⁺, iPSC-derived cardiomyocytes were loaded with 5 μM fluorescent Ca²⁺ indicator Fluo-4 AM (catalog # F14201; Thermo Fisher Scientific) in the presence of 2 mmol/L of Pluronic F-127 for 20 min in Tyrode's solution (140 mM NaCl, 5.4 mM KCl, 1 mM MgCl₂, 10 mM glucose, 1.8 mM CaCl₂, 10 mM HEPES, pH 7.4). The Ca²⁺ transients of spontaneously beating iPSC-derived cardiomyocytes were imaged at 37° C. using a Zeiss LSM880 confocal system using 40× oil immersion objective. Ca²⁺ transients were processed and analyzed using ImageJ (NIH, Bethesda) and pClamp 10.2 software (Axon Instrument). For the calcium release phase, the inventors calculated time to peak (time from baseline to maximal point of the transient). For evaluation of calcium re-uptake, the total transient time and time of Ca²⁺ decay were evaluated. The time of Ca²⁺ decay was calculated by fitting a first-order exponential decay to the calcium reuptake phase of the calcium transient profile. Cells were considered arrhythmic if they had one or more asynchronized Ca²⁺ transient. For each experimental group, the inventors analyzed cells from at least three independent sets of iPSC-derived cardiomyocytes.

Engineered Heart Muscle.

Engineered heart muscle (EHM) was constructed as previously reported from iPSC-derived cardiomyocytes of iDMD (ΔEx8-9; n=6) and the corrected lines (ΔEx3-9, n=14; ΔEx6-9, n=3; ΔEx7-11, n=3). All lines were adapted to a published cardiomyocyte differentiation protocol, which resulted in high and homogeneous cardiomyocyte purity in the different iPSC cultures, showing 95±1% α-actinin positive (ACTN2⁺) cardiomyocytes by flow cytometry; n=8. For EHM construction, 1×10⁶ iPSC-derived cardiomyocytes were mixed with 0.5×10⁶ human foreskin fibroblasts (catalog #SCRC-1041; ATCC). EHM was cultured for 4 weeks followed by isometric force measurements under electrical field stimulation (1.5 Hz) at 37° C. in Tyrode's solution. Two healthy donor-derived iPSC lines were included as a reference: control (WT) shiPS9-1 (Daniel Garry, University of Minnesota, Minneapolis, Minn.) (n=4) and hiPSC-G1 line (n=4).

CRISPR/Cas9-Mediated Exon Deletion in Mice.

Two single-guide RNA (sgRNA) specific regions of introns 7 and 9, surrounding exons 8 and 9 in the sequence of the mouse Dmd locus, were cloned into vector pX458. For the in vitro transcription of sgRNA, T7 promoter sequence was added to the sgRNA template by PCR. The gel purified PCR products were used as template for in vitro transcription using the MEGAshortscript T7 Kit (Life Technologies). sgRNA were purified by MEGAclear kit (Life Technologies) and eluted with nuclease-free water (Ambion). The concentration of guide RNA was measured by a NanoDrop instrument (Thermo Scientific).

Genotyping of ΔEx8-9 DMD Mice.

ΔEx8-9 DMD mice were genotyped using primers encompassing the targeted region as follows:

(SEQ ID NO: 90)
mDMD_i7_forward: 5′-GAGGTTTAAAACATTAAGCCTTTCC-3′
(SEQ ID NO: 91)
mDMD_i7_reverse: 5′-ACATTAAGATGGACTTCTTGTCTGG-3′
(SEQ ID NO: 92)
mDMD_i9_reverse: 5′-TACTCACATATGGGTCGTTTTCCTT-3′.

Tail biopsies were digested in 100 μL of 25 mM NaOH, 0.2 mM EDTA (pH 12) for 20 min at 95° C. Tails were briefly centrifuged followed by addition of 100 μL of 40 mM Tris.HCl (pH 5) and mixed to homogenize. Two microliters of this reaction was used for subsequent PCR reactions with the primers below, followed by gel electrophoresis.

Histological Analysis of Muscles.

Skeletal muscles from WT and ΔEx8-9 DMD mice were individually dissected and cryoembedded in a 1:2 volume mixture of Gum Tragacanth powder (Sigma-Aldrich) to Tissue Freezing Medium (TFM) (Triangle Bioscience). All embeds were snap frozen in isopentane heat extractant supercooled to −155° C. Resulting blocks were stored overnight at −80° C. prior to sectioning. Eight-micron transverse sections of skeletal muscle, and frontal sections of heart were prepared on a Leica CM3050 cryostat and air-dried prior to same day staining. H&E staining was performed according to established staining protocols and dystrophin immunohistochemistry was performed using MANDYS8 monoclonal antibody (Sigma-Aldrich) with modifications to manufacturer's instructions. In brief, cryostat sections were thawed and rehydrated/delipidated in 1% triton/phosphate-buffered-saline, pH 7.4 (PBS). Following delipidation, sections were washed free of Triton, incubated with mouse IgG blocking reagent (M.O.M. Kit, Vector Laboratories), washed, and sequentially equilibrated with MOM protein concentrate/PBS, and MANDYS8 diluted 1:1800 in MOM protein concentrate/PBS. Following overnight primary antibody incubation at 4° C., sections were washed, incubated with MOM biotinylated anti-mouse IgG, washed, and detection completed with incubation of Vector fluorescein-avidin DCS. Nuclei were counterstained with propidium iodide (Molecular Probes) prior to cover slipping with Vectashield.

Statistical Analysis.

Statistical analysis was assessed by one-way ANOVA or two-way ANOVA and Tukey's post-hoc test (F) were indicated. Data are shown as mean±SEM. A P<0.05 value was considered statistically significant.

### Example 2—Results

Generating a Dystrophin Mutation that Disrupts the Actin-Binding Domain.

The inventors generated a human iPSC line with a deletion of exons 8 and 9 in the DMD gene using CRISPR/Cas9-mediated editing to analyze the effect ABD-1 deletions on muscle function (FIG. 1A). Two single guide RNAs (gRNAs), targeting sequences in intron 7 and intron 9 of the DMD gene in the presence of Streptococcus pyogenes Cas9, were used to delete DMD exons 8-9 in healthy human iPSCs (FIG. 1B). This induced DMD mutant human cell line is referred to as ΔEx8-9 iDMD, and represents the common 5′-proximal hot spot mutations. Human iPSCs harboring DMD exons 8-9 deletions were picked by clonal selection and the mutation was confirmed by sequencing genomic DNA (FIG. 1C). The human ΔEx8-9 iDMD iPSC line has a 1,909 bp deletion, which generates a newly formed junction between intron 7 to intron 9 (FIG. 1C). Primers within intron 7 flanking the gRNA-7 targeting site were used to validate the deletion and as expected showed no PCR product with ΔEx8-9 iDMD compared to control iPSC with a 424 bp product (FIG. 1C). The ΔEx8-9 iDMD iPSCs were differentiated to cardiomyocytes and RT-PCR was performed using forward and reverse primers targeting exons 1 and 10, respectively (FIG. 1D). Sequencing of the RT-PCR products showed splicing of exon 7 to exon 10, which created a premature stop codon following the first 5 amino acids encoded by exon 10 (FIG. 1D). Loss of dystrophin protein was confirmed by Western blot analysis and immunocytochemistry in ΔEx8-9 iDMD iPSC-derived cardiomyocytes (FIGS. 1E-F).

Correction of the ΔEx8-9 iDMD Mutation by Exon Deletion Strategies.

Three different CRISPR/Cas9-mediated strategies were used to correct the mutation in ΔEx8-9 iDMD iPSCs in order to restore the dystrophin open reading frame (FIG. 2A). These strategies generated dystrophin with different truncations in the ABD-1 domain, providing a means of analyzing the effect of ABD-1 deletions on muscle function. For each strategy, the inventors applied two gRNAs to delete multiple exons and analyzed two independent iPSC clones. Strategy #1 generated ΔEx3-9 iPSCs, by targeting intron 2 and intron 7 with gRNAs, and deleting exons 3 through 7 (FIG. 2B). Genomic sequencing of the genome edited region showed a hybrid junction of intron 2 to intron 7, confirming generation of ΔEx3-9 iPSCs (FIG. 2C). Following differentiation of ΔEx3-9 iPSCs to cardiomyocytes, RT-PCR was performed using primers within exon 1 and exon 10 (FIG. 2D). Sequencing of the RT-PCR product showed splicing of exon 2 to exon 10. Restoration of the DMD open reading frame generated a truncated dystrophin protein lacking the ABS-2 and ABS-3 regions. Dystrophin expression was confirmed by Western blot analysis and immunocytochemistry of ΔEx3-9 iPSC-derived cardiomyocytes (FIGS. 2E-F; FIG. 7A).

Strategy #2 generated ΔEx6-9 iPSCs, using gRNAs targeting intron 5 and intron 7 in order to delete exons 6 and 7 (FIG. 3A). This form of CRISPR/Cas9 editing resulted in deletion of exon 6 through exon 9, which was confirmed by sequencing of genomic PCR products (FIG. 3B). Following differentiation, RT-PCR using primers within exon 1 and exon 10 was performed on ΔEx6-9 iPSC-derived cardiomyocytes and sequencing of the RT-PCR product confirmed splicing of exon 5 to exon 10 (FIG. 3C). Restoration of dystrophin expression generated a truncated protein lacking the ABS-3 region. Dystrophin expression was confirmed by Western blot analysis and immunocytochemistry in ΔEx6-9 iPSC-derived cardiomyocytes (FIGS. 3D-E; FIGS. 7A-B).

Strategy #3 generated ΔEx7-11 iPSCs, using gRNAs targeting introns 6 and 11 to delete exons 7 to 11 of ΔEx8-9 iDMD iPSCs (FIG. 4A). This genome editing strategy introduced the largest deletion of 164 kb in ΔEx8-9 iDMD iPSCs. Similar to the first two correction strategies, single cell-derived colonies were picked from the corrected iPSC pool and the genomic region was sequenced to confirm editing (FIG. 4B). The ΔEx7-11 iPSCs were differentiated to cardiomyocytes, and RT PCR was performed with primers within exon 5 and exon 12 to reveal splicing of exon 6 to exon 12, as seen by sequencing (FIG. 4C). Restoration of the DMD open reading frame of ΔEx7-11 iPSC-derived cardiomyocytes generated a truncated dystrophin protein lacking amino acids 177-444 but with all three ABS intact. Dystrophin expression was confirmed by Western blot analysis and immunocytochemistry (FIGS. 4D-E; FIGS. 7A-B). The inventors observed less dystrophin expression in ΔEx7-11 iPSC-derived cardiomyocytes compared to control iPSC-derived cardiomyocytes and the other corrected lines, ΔEx3-9 and ΔEx6-9. Previously, it was shown that missense mutations associated with the DMD phenotype caused steady-state decreases in dystrophin protein levels inversely proportional to the tertiary stability and directly caused by proteasomal degradation. Consistent with this report, the inventors observed increased dystrophin protein levels when ΔEx7-11 iPSC-derived cardiomyocytes were treated with the proteasome inhibitor MG-132, as measured by Western blot analysis (FIG. 4F; FIG. 7C). This suggests that deletion of DMD exons 7-11, which produces dystrophin lacking 177-444 amino acids, results in a truncated form of dystrophin that is degraded.

Functional Restoration of iPSC-Derived Cardiomyocytes by Correction of ABD-1 Mutations.

To assess the consequences of ABD-1 mutations and the effect of correction by genome editing on dystrophin function in muscle, the inventors analyzed spontaneous Ca²⁺ activity in iPSC-derived cardiomyocytes (FIG. 5A). For each CRISPR/Cas9-edited correction, the inventors analyzed data sets from two corrected clones. As expected, the inventors observed that calcium release and reuptake parameters, including time to peak, Ca²⁺ decay rate, and transient duration were significantly higher in the ΔEx8-9 iDMD iPSC-derived cardiomyocytes compared to isogenic control cells (FIGS. 5B-D). Additionally, greater than 50% of the ΔEx8-9 iDMD iPSC-derived cardiomyocytes demonstrated abnormal Ca²⁺ activity, which represents an arrhythmogenic-like phenotype (FIG. 5E). In contrast, only 10.7% of the control iPSC-derived cardiomyocytes were desynchronized (FIG. 5E).

Corrected ΔEx3-9 iPSC-derived cardiomyocytes displayed normalized Ca²⁺ transient kinetics and decreased asynchronous activity similar to isogenic control cells, suggesting restoration of dystrophin function (FIGS. 5A-G). Cardiomyocytes derived from ΔEx6-9 iPSCs showed improved Ca²⁺ handling, although not to the level seen with ΔEx3-9 cells (FIGS. 5A-G). The Ca²⁺ decay rate and Ca²⁺ transient duration were shorter in ΔEx6-9 iPSC-derived cardiomyocytes (FIGS. 5B and 5D) and the number of arrhythmic cells was decreased to 29% compared to ΔEx8-9 iDMD cells (FIG. 5E). However, the parameters observed in the ΔEx6-9 iPSC-derived cardiomyocytes did not reach the levels of control iPSC-derived cardiomyocytes. The ΔEx7-11 iPSC-derived cardiomyocytes, similarly to ΔEx6-9 cells, showed improvement in Ca²⁺ release and uptake when compared to ΔEx8-9 iDMD cardiomyocytes (FIGS. 5B and 5C), however they did not reach control cell levels. iPSC-derived cardiomyocytes with ΔEx7-11 had the highest number of arrhythmic cells, up to 35.9%, among the corrected cell lines (FIG. 5E). Taken together, these findings indicate that deletion of exons 3-9 (ΔEx3-9) to correct DMD mutations within the 5′-proximal hot spot of the DMD gene produces a truncated dystrophin protein with restoration of function.

Further assessment of the functional properties of the iPSC-derived cardiomyocyte cell lines was performed by generating engineered heart muscle (EHM), which displays phenotypic properties of the postnatal myocardium. EHM was generated as shown in FIG. 8A using ˜95% pure iPSC-derived cardiomyocytes, as measured by α-actinin expression (FIG. 8B). Consistent with the calcium handling data, the inventors observed enhanced contractile performance in all corrected cell lines, with the most pronounced effects in ΔEx3-9 EHM (FIG. 5F). Moreover, the inventors observed an increased tendency for arrhythmic contractions in ΔEx8-9 iDMD-EHM and a reduction in ΔEx3-9 EHM (FIG. 8C).

Correction of DMD Patient-Derived iPSCs by Deleting Exons 3-9.

An iPSC line, referred to as pΔEx3-7, was generated from a DMD patient (SC604A-MD) with a deletion of exons 3-7 in the DMD gene. pΔEx3-7 iPSCs are used as patient-in-a-dish model of DMD with a mutation in the ABD-1. To evaluate the effectiveness of exon 3-9 deletion in restoration of cardiomyocyte function, the inventors corrected pΔEx3-7 by deleting exons 8 and 9 to generate pΔEx3-9. Two gRNAs targeting introns 7 and 9 were used to excise exons 8-9, generating pΔEx3-9 iPSCs (FIG. 6A). Sequencing of genomic DNA PCR products confirmed the hybrid junction of intron 7 to intron 9 in a single cell-derived iPSC colony (FIG. 6B). Both pΔEx3-7 and pΔEx3-9 iPSC lines were differentiated into cardiomyocytes and RT-PCR was performed with primers targeting the 5′-UTR and exon 10. In pΔEx3-7 iPSC-derived cardiomyocytes, the junction of exon 2 to exon 8 was seen by sequencing of the RT-PCR product (FIG. 6C). In corrected pΔEx3-9 iPSC-derived cardiomyocytes, the junction of exon 2 to exon 10 was demonstrated by sequencing the RT-PCR product (FIG. 6C). Western blot analysis showed a low level of dystrophin expression in DMD pΔEx3-7 iPSC-derived cardiomyocytes (FIG. 6D). This is consistent with previous reports showing low levels of dystrophin protein expression in patients lacking exons 3-7 due to re-initiation of translation at an in-frame start codon in exon 8 (Fletcher et al., 2012; Winnard et al., 1995). Corrected pΔEx3-9 iPSC-derived cardiomyocytes showed dystrophin expression similar to normal control cardiomyocytes (FIG. 6D). These findings were confirmed by immunocytochemistry (FIG. 6E).

Spontaneous Ca²⁺ activity was assessed in these patient-derived cells as a measure of iPSC-derived cardiomyocyte functionality (FIGS. 6F-I). In pΔEx3-7 DMD iPSC-derived cardiomyocytes, Ca²⁺ time to peak and time to half decay were elevated (FIGS. 6F-G). This caused an overall slower Ca²⁺ transient (FIG. 6H) and elevated the number of arrhythmic cells, up to 53% (FIG. 6I). Corrected pΔEx3-9 iPSC-derived cardiomyocytes had significantly improved time to peak and faster Ca²⁺ decay (FIGS. 6F-G). Although these parameters of the corrected pΔEx3-9 iPSC-derived cardiomyocytes did not reach normal control levels, the number of arrhythmic cells was 18.5%, similar to control iPSC-derived cardiomyocytes (FIG. 6I). These findings show improved iPSC-derived cardiomyocyte function after genomic editing to excise exons 3-9 of the DMD gene in DMD patient-derived iPSCs.

ΔEx8-9 DMD Mouse Models Recapitulate Muscle Dystrophy Phenotype.

To investigate CRISPR/Cas9-mediated exon skipping and reframing in vivo, a mimic of the human ABD-1 region mutations was generated in a mouse model by deleting exons 8 and 9, using CRISPR/Cas9 system directed by two single guide RNAs (sgRNA) (FIG. 9A and Table 2). The inventors designed and validated sgRNAs targeting introns, flanking 5′ end and 3′ ends of Dmd exons 8 and 9 respectively. C57BL/6 zygotes were co-injected with in vitro transcribed Cas9 mRNA and in vitro transcribed sgRNAs, and then re-implanted into pseudo-pregnant females.

The deletion of Dmd exons 8-9 was confirmed by DNA genotyping. Mice lacking exons 8-9 showed pronounced dystrophic muscle changes in 1 and 2 month-old mice (FIG. 9B). The deletion of these exons placed the dystrophin gene out of frame leading to the absence of dystrophin protein in skeletal muscle and heart (FIG. 9C). The grip strength of the ΔEx8-9 DMD was significantly weaker than that in wild-type mice (FIG. 9D). Serum analysis of the ΔEx8-9 DMD mice shows a significant increase of creatine kinase (CK) level, which is a sign of muscle damage (FIG. 9E). Taken together, dystrophin protein expression, muscle histology, muscle strength and serum creatine kinase level validated the dystrophic phenotype of the ΔEx8-9 DMD mouse model.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference.
Angel et al., Mol. Cell. Biol., 7:2256, 1987a.
Angel et al., Cell, 49:729, 1987b.
Aartsma-Rus et al., Hum. Mutat. 30, 293-299, 2009.
Baichwal and Sugden, In: Gene Transfer, Kucherlapati (Ed), NY, Plenum Press, 117-148, 1986.
Banerji et al., Cell, 27(2 Pt 1):299-308, 1981.
Banerji et al., Cell, 33(3):729-740, 1983.
Barnes et al., J Biol. Chem., 272(17):11510-7, 1997.
Baskin et al., EMBO Mol Med 6, 1610-1621, 2014.
Benvenisty and Neshif, Proc. Natl. Acad. Sci. USA, 83:9551-9555, 1986.
Berkhout et al., Cell, 59:273-282, 1989.
Bhavsar et al., Genomics, 35(1): 11-23, 1996.
Bikard et al., Nucleic Acids Res. 41(15): 7429-7437, 2013.
Blanar et al., EMBO J., 8:1139, 1989.
Bodine and Ley, EMBO J., 6:2997, 1987.
Boshart et al., Cell, 41:521, 1985.
Bostick et al., Mol Ther 19, 1826-1832, 2011.
Bosze et al., EMBO J., 5(7):1615-1623, 1986.
Braddock et al., Cell, 58:269, 1989.
Brinster et al., Proc. Natl. Acad. Sci. USA, 82(13):4438-4442, 1985.
Bulla and Siddiqui, J. Virol., 62:1437, 1986.
Burridge et al., Nat. Methods 11, 855-860, 2014.
Bushby et al., Lancet Neurol., 9(1): 77-93 (2010).
Bushby et al., Lancet Neurol., 9(2): 177-198 (2010).
Campbell & Kahl, Nature 338, 259-262, 1989.
Campbell and Villarreal, Mol. Cell. Biol., 8:1993, 1988.
Campere and Tilghman, Genes and Dev., 3:537, 1989.
Campo et al., Nature, 303:77, 1983.
Celander and Haseltine, J. Virology, 61:269, 1987.
Celander et al., J. Virology, 62:1314, 1988.
Chandler et al., Cell, 33:489, 1983.
Chang et al., Mol. Cell. Biol., 9:2153, 1989.
Chang et al., Stem Cells., 27:1042-1049, 2009.
Chatterjee et al., Proc. Natl. Acad. Sci. USA, 86:9114, 1989.
Chen and Okayama, Mol. Cell Biol., 7:2745-2752, 1987.
Cho et al., Nat. Biotechnol. 31(3): 230-232, 2013.
Choi et al., Cell, 53:519, 1988.
Cirak et al., The Lancet 378, 595-605, 2011.
Coffin, In: Virology, Fields et al. (Eds.), Raven Press, NY, 1437-1500, 1990.
Cohen et al., J Cell. Physiol., 5:75, 1987.
Costa et al., Mol. Cell. Biol., 8:81, 1988.
Couch et al., Am. Rev. Resp. Dis., 88:394-403, 1963.
Coupar et al., Gene, 68:1-10, 1988.
Cripe et al., EMBO J., 6:3745, 1987.
Culotta and Hamer, Mol. Cell. Biol., 9:1376, 1989.
Dandolo et al., J. Virology, 47:55-64, 1983.
De Villiers et al., Nature, 312(5991):242-246, 1984.
Deschamps et al., Science, 230:1174-1177, 1985.
DeWitt et al., Sci Transl Med 8, 360ra134-360ra134, 2016.
Donnelly et al., J. Gen. Virol. 82, 1027-1041, 2001.
Dubensky et al., Proc. Natl. Acad. Sci. USA, 81:7529-7533, 1984.
Edbrooke et al., Mol. Cell. Biol., 9:1908, 1989.
Edlund et al., Science, 230:912-916, 1985.
EP 0273085
Fechheimer et al., Proc Natl. Acad. Sci. USA, 84:8463-8467, 1987.
Feng and Holland, Nature, 334:6178, 1988.
Ferkol et al., FASEB J., 7:1081-1091, 1993.
Firak et al., Mol. Cell. Biol., 6:3667, 1986.
Foecking et al., Gene, 45(1):101-105, 1986.
Fonfara et al., Nature 532, 517-521, 2016.
Fraley et al., Proc Natl. Acad. Sci. USA, 76:3348-3352, 1979
Franz et al., Cardoscience, 5(4):235-43, 1994.
Friedmann, Science, 244:1275-1281, 1989.
Fujita et al., Cell, 49:357, 1987.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Ghosh-Choudhury et al., EMBO J., 6:1733-1739, 1987.
Gilles et al., Cell, 33:717, 1983.
Gloss et al., EMBO J., 6:3735, 1987.
Godbout et al., Mol. Cell. Biol., 8:1169, 1988.
Gomez-Foix et al., J. Biol. Chem., 267:25129-25134, 1992.
Goncalves et al., Mol Ther, 19(7): 1331-1341 (2011).
Goodbourn and Maniatis, Proc. Natl. Acad. Sci. USA, 85:1447, 1988.
Goodboum et al., Cell, 45:601, 1986.
Gopal, Mol. Cell Biol., 5:1188-1190, 1985.
Gopal-Srivastava et al., J Mol. Cell. Biol., 15(12):7081-90, 1995.
Graham and Prevec, In: Methods in Molecular Biology: Gene Transfer and Expression Protocol, Murray (Ed.), Humana Press, Clifton, N.J., 7:109-128, 1991.
Graham and van der Eb, Virology, 52:456-467, 1973.
Graham et al., J Gen. Virol., 36:59-72, 1977.
Greene et al., Immunology Today, 10:272, 1989
Grosschedl and Baltimore, Cell, 41:885, 1985.
Grunhaus and Horwitz, Seminar in Virology, 3:237-252, 1992.
Harland and Weintraub, J. Cell Biol., 101:1094-1099, 1985.
Haslinger and Karin, Proc. Natl. Acad. Sci. USA, 82:8572, 1985.
Hauber and Cullen, J. Virology, 62:673, 1988.
Hen et al., Nature, 321:249, 1986.
Hensel et al., Lymphokine Res., 8:347, 1989.
Hermonat and Muzycska, Proc. Nat'l Acad. Sci. USA, 81:6466-6470, 1984.
Herr and Clarke, Cell, 45:461, 1986.
Hersdorffer et al., DNA Cell Biol., 9:713-723, 1990.
Herz and Gerard, Proc. Nat'l. Acad. Sci. USA 90:2812-2816, 1993.
Hirochika et al., J. Virol., 61:2599, 1987.
Holbrook et al., Virology, 157:211, 1987.
Hollinger & Chamberlain, Current Opinion in Neurology 28, 522-527, 2015.
Horlick and Benfield, Mol. Cell. Biol., 9:2396, 1989.
Horwich et al., J. Virol., 64:642-650, 1990.
Hsu et al., Natl Biotechnol. 31:827-832, 2013
Huang et al., Cell, 27:245, 1981.
Hug et al., Mol. Cell. Biol., 8:3065, 1988.
Hwang et al., Mol. Cell. Biol., 10:585, 1990.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Jinek et al., Science 337, 816-821, 2012.
Johnson et al., Mol. Cell. Biol., 9:3393, 1989.
Jones and Shenk, Cell, 13:181-188, 1978.
Kadesch and Berg, Mol. Cell. Biol., 6:2593, 1986.
Kaneda et al., Science, 243:375-378, 1989.
Karin et al., Mol. Cell. Biol., 7:606, 1987.
Karlsson et al., EMBO J., 5:2377-2385, 1986.
Katinka et al., Cell, 20:393, 1980.
Kato et al., J Biol Chem., 266(6):3361-3364, 1991.
Kawamoto et al., Mol. Cell. Biol., 8:267, 1988.
Kelly et al., J. Cell Biol., 129(2):383-96, 1995.
Kiledjian et al., Mol. Cell. Biol., 8:145, 1988.
Kim et al., Nature Biotechnology, 1-2, 2016.
Kim et al., Nature Biotechnology 34, 876-881, 2016.
Kimura et al., Dev. Growth Differ., 39(3):257-65, 1997.
Klamut et al., Mol. Cell. Biol., 10:193, 1990.
Klein et al., Nature, 327:70-73, 1987.
Koch et al., Mol. Cell. Biol., 9:303, 1989.
Kriegler and Botchan, In: Eukaryotic Viral Vectors, Gluzman (Ed.), Cold Spring Harbor: Cold Spring Harbor Laboratory, N Y, 1982.
Kriegler and Botchan, Mol. Cell. Biol., 3:325, 1983.
Kriegler et al., Cell, 38:483, 1984.
Kriegler et al., Cell, 53:45, 1988.
Kuhl et al., Cell, 50:1057, 1987.
Kunz et al., Nucl. Acids Res., 17:1121, 1989.
LaPointe et al., Hypertension, 27(3):715-22, 1996
LaPointe et al., J. Biol. Chem., 263(19):9075-8, 1988.
Larsen et al., Proc. Natl. Acad. Sci. USA., 83:8283, 1986.
Laspia et al., Cell, 59:283, 1989.
Latimer et al., Mol. Cell. Biol., 10:760, 1990.
Le Gal La Salle et al., Science, 259:988-990, 1993.
Lee et al., Nature, 294:228, 1981.
Levinson et al., Nature, 295:79, 1982.
Levrero et al., Gene, 101:195-202, 1991.
Lin et al., Mol. Cell. Biol., 10:850, 1990.
Long et al., Science 345: 1184-1188, 2014.
Long et al., Science 351, 400-403, 2016.
Long et al., JAMA Neurol., 3388, 2016.
Long et al., Science 351, 400-403, 2016.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Majors and Varmus, Proc. Natl. Acad. Sci. USA, 80:5866, 1983.
Mali et al., Science 339, 823-826, 2013a.
Mali et al., Nat Methods 10, 957-963, 2013b.
Mali et al., Nat. Biotechnol. 31:833-838, 2013c.
Mann et al., Cell, 33:153-159, 1983.
Maresca et al., Genome Research 23, 539-546, 2013.
Markowitz et al., J. Virol., 62:1120-1124, 1988.
McNeall et al., Gene, 76:81, 1989.
Miksicek et al., Cell, 46:203, 1986.
Millay et al., Nat. Med. 14, 442-447, 2008.
Mojica et al., J. Mol. Evol. 60, 174-182, 2005.
Mordacq and Linzer, Genes and Dev., 3:760, 1989.
Moreau et al., Nucl. Acids Res., 9:6047, 1981.
Moss et al., J. Gen. Physiol., 108(6):473-84, 1996.
Muesing et al., Cell, 48:691, 1987.
Nelson et al., Science 351, 403-407, 2016.
Nicolas and Rubinstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Ondek et al., EMBO J., 6:1017, 1987.
Omitz et al., Mol. Cell. Biol., 7:3466, 1987.
Padgett, R. A., Trends Genet. 28, 147-154, 2012.
Palmiter et al., Cell, 29:701, 1982a.
Palmiter et al., Nature, 300:611, 1982b.
Paskind et al., Virology, 67:242-248, 1975.
Pech et al., Mol. Cell. Biol., 9:396, 1989.
Perales et al., Proc. Natl. Acad. Sci. USA, 91(9):4086-4090, 1994.
Perez-Stable and Constantini, Mol. Cell. Biol., 10:1116, 1990.
Picard et al., Nature, 307:83, 1984.
Pinkert et al., Genes and Dev., 1:268, 1987.
Ponta et al., Proc. Natl. Acad. Sci. USA, 82:1020, 1985.
Porton et al., Mol. Cell. Biol., 10:1076, 1990.
Potter et al., Proc. Natl. Acad. Sci. USA, 81:7161-7165, 1984.
Queen and Baltimore, Cell, 35:741, 1983.
Quinn et al., Mol. Cell. Biol., 9:4713, 1989.
Racher et al., Biotech. Techniques, 9:169-174, 1995.
Ragot et al., Nature, 361:647-650, 1993.
Ran et al., Nature 520, 186-191, 2015.
Redondo et al., Science, 247:1225, 1990.
Reisman and Rotter, Mol. Cell. Biol., 9:3571, 1989.
Renan, Radiother. Oncol., 19:197-218, 1990.
Resendez Jr. et al., Mol. Cell. Biol., 8:4579, 1988.
Rich et al., Hum. Gene Ther., 4:461-476, 1993.
Ridgeway, In: Vectors: A survey of molecular cloning vectors and their uses, Stoneham: Butterworth, 467-492, 1988.
Ripe et al., Mol. Cell. Biol., 9:2224, 1989.
Rippe et al., Mol. Cell Biol., 10:689-695, 1990.
Rittling et al., Nuc. Acids Res., 17:1619, 1989.
Rosen et al., Cell, 41:813, 1988.
Rosenfeld et al., Cell, 68:143-155,1992.
Rosenfeld et al., Science, 252:431-434,1991.
Roux et al., Proc. Natl. Acad. Sci. USA, 86:9079-9083, 1989.
Sambrook and Russell, Molecular Cloning: A Laboratory Manual 3^{rd} Ed., Cold Spring Harbor Laboratory Press, 2001.
Sakai et al., Genes and Dev., 2:1144, 1988.
Satake et al., J. Virology, 62:970, 1988.
Schaffner et al., J. Mol. Biol., 201:81, 1988.
Searle et al., Mol. Cell. Biol., 5:1480, 1985.
Sharp et al., Cell, 59:229, 1989.
Shaul and Ben-Levy, EMBO J., 6:1913, 1987.
Sherman et al., Mol. Cell. Biol., 9:50, 1989.
Sleigh et al., J. EMBO, 4:3831, 1985.
Shimizu-Motohashi et al., Am J Transl Res 8, 2471-89, 2016.
Spalholz et al., Cell, 42:183, 1985.
Spandau and Lee, J. Virology, 62:427, 1988.
Spandidos and Wilkie, EMBO J., 2:1193, 1983.
Stephens and Hentschel, Biochem. J., 248:1, 1987.
Stratford-Perricaudet and Perricaudet, In: Human Gene Transfer, Cohen-Haguenauer and Boiron (Eds.), John Libbey Eurotext, France, 51-61, 1991.
Stratford-Perricaudet et al., Hum. Gene. Ther., 1:241-256, 1990.
Stuart et al., Nature, 317:828, 1985.
Sullivan and Peterlin, Mol. Cell. Biol., 7:3315, 1987.
Swartzendruber and Lehman, J. Cell. Physiology, 85:179, 1975.
Tabebordbar et al., Science 351, 407-411, 2016.
Takebe et al., Mol. Cell. Biol., 8:466, 1988.
Tavemier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Temin, In: Gene Transfer, Kucherlapati (Ed.), NY, Plenum Press, 149-188, 1986.
Thiesen et al., J. Virology, 62:614, 1988.
Top et al., J. Infect. Dis., 124:155-160, 1971.
Tóth et al., Biology Direct, 1-14, 2016.
Tronche et al., Mol. Biol. Med., 7:173, 1990.
Trudel and Constantini, Genes and Dev. 6:954, 1987.
Tsai et al., Nature Biotechnology 34, 882-887, 2016.
Tur-Kaspa et al., Mol. Cell Biol., 6:716-718, 1986.
Tyndell et al., Nuc. Acids. Res., 9:6231, 1981.
Vannice and Levinson, J. Virology, 62:1305, 1988.
Varmus et al., Cell, 25:23-36, 1981.
Vasseur et al., Proc Natl. Acad. Sci. U.S.A., 77:1068, 1980.
Wagner et al., Proc. Natl. Acad. Sci. USA, 87(9):3410-3414, 1990.
Walmsley et al., PLoS ONE 5, e8647, 2010.
Wang and Calame, Cell, 47:241, 1986.
Wang et al., Cell, 153:910-910, 2013.
Weber et al., Cell, 36:983, 1984.
Weinberger et al. Mol. Cell. Biol., 8:988, 1984.
Winoto et al., Cell, 59:649, 1989.
Wong et al., Gene, 10:87-94, 1980.
Wu and Wu, Adv. Drug Delivery Rev., 12:159-167, 1993.
Wu and Wu, Biochemistry, 27:887-892, 1988.
Wu and Wu, J. Biol. Chem., 262:4429-4432, 1987.
Wu et al., Cell Stem Cell 13, 659-662, 2013.
Wu et al., Nat Biotechnol 32, 670-676, 2014.
Xu et al., Mol Ther 24, 564-569, 2016.
Yamauchi-Takihara et al., Proc. Natl. Acad. Sci. USA, 86(10):3504-3508, 1989.
Yang et al., Proc. Natl. Acad. Sci. USA, 87:9568-9572, 1990.
Yin et al., Nat Biotechnol 32, 551-553, 2014.
Yin et al., Physiol Rev 93, 23-67, 2013.
Young et al., Cell Stem Cell 18, 533-540, 2016.
Yutzey et al. Mol. Cell. Biol., 9:1397, 1989.
Zechner et al., Cell Metabolism 12, 633-642, 2010.
Zelenin et al., FEBS Lett., 280:94-96, 1991.
Zetsche et al., Cell 163, 759-771, 2015.
Ziober and Kramer, J. Bio. Chem., 271(37):22915-22922, 1996.
Campbell and Kahl, 338(6212):259-62, 1989.
Le Rumeur E. Bosn J Basic Med Sci. 15(3): 14-20, 2015.
Monaco et al., xdgGenomics 2(1):90-5, 1988.
Allen et al., Physiol Rev. 96(1):253-305, 2016.
Kyrychenko et al., Cardiovasc Res. 97(4):666-75, 2013.
Beggs et al., Am J Hum Genet. 49(1):54-67, 1991.
Aartsma-Rus et al., Muscle Nerve. 34(2): 135-44, 2006.
Tuffery-Giraud et al., Hum Mutat. 30(6):934-45, 2009.
Bladen et al., Hum Mutat. 36(4):395-402, 2015.
Yang et al., BMC Med Genet. 14:29, 2013.
Nakamura et al., J Clin Neurosci. 15(7):757-63, 2008.
Bengtsson et al., Nat Commun. 8:14454, 2017.
Li et al., Stem Cell Reports 4(1):143-54, 2015.
Long et al., Science 351(6271):400-3, 2016.
Long et al., Science 345(6201):1184-8, 2014.
Ousterout et al., Nat Commun. 6(6244), 2015.
Yu et al., Science Advances 3(4):e1602814, 2017.
Gregorevic et al., Nat Med. 12(7):787-9, 2006.
Sakamoto et al., Biochem Biophys Res Commun. 293(4): 1265-72, 2002.
Yue et al., Hum Mol Genet. 24(20):5880-90, 2015.
Toh et al., PLoS One 11(1):e0145620, 2016.
Young et al., Cell Stem Cell. 18(4):533-40, 2016.
Tabebordbar et al. Science 351(6271):407-11, 2016.
Nelson et al., Science 351(6271):403-7, 2016.
Xu et al., Mol Ther. 24(3):564-9, 2016.
Corrado et al., FEBS Lett. 344(2-3):255-60, 1994.
Henderson et al., Proc Natl Acad Sci USA 107(21):9632-7, 2010.
Levine et al., FEBS Lett. 298(1):44-8, 1992.
Sutherland-Smith et al., J Mol Biol. 329(1): 15-33, 2003.
Wein et al., Nat Med. 20(9):992-1000, 2014.
Lattanzi et al., Mol Ther Nucleic Acids 7:11-9, 2017.
Bish et al., Mol Ther. 20(3):580-9, 2012.
Echigoya et al., Proc Natl Acad Sci USA 114(16):4213-8, 2017.
Kaspar et al., Circ Cardiovasc Genet. 2(6):544-51, 2009.
Novakovic et al., Ann NY Acad Sci. 1048:406-10, 2005.
Singh et al., Proc Natl Acad Sci USA 107(34):15069-74, 2010.
Heald et al., Neurology 44(12):2388-90, 1994.
Nakamura et al., J Hum Genet. 61(7):663-7, 2016.
Matsa et al., Physiol Rev. 96(3): 1093-126, 2016.
Tiburcy et al., Circulation. 135(19): 1832-47, 2017.
Talsness et al., Proc Natl Acad Sci USA 112(40): 12414-9, 2015.
Fletcher et al., Mol Ther Nucleic Acids 1: e48, 2012.
Winnard et al., Am J Hum Genet. 56(1): 158-66, 1995.
Guan et al., Stem Cell Res. 12(2):467-80, 2014.
Lin et al., Dis Model Mech. 8(5):457-66, 2015.
Rajdev and Groh, Card Electrophysiol Clin. 7(2):303-8, 2015.
Yumlu et al., Methods. 121-122:29-44, 2017.
Veres et al., Cell Stem Cell 15(1):27-30, 2014.
Yang et al., Nat Commun. 5:5507, 2014.
Aartsma-Rus et al., J Med Genet. 53(3): 145-51, 2016.
Odom et al., Mol Ther. 19(1):36-45, 2011.
Banks et al., Hum Mol Genet. 16(17):2105-13, 2007.
Acsadi et al., J Biol Chem. 287(22):18153-62, 2012.
Corrado et al., J Cell Biol. 134(4):873-84, 1996.
van Putten et al., PLoS One 7(2):e31937, 2012.
Wells et al., Hum Mol Genet. 4(8):1245-50, 1995.
Neri et al., Neuromuscul Disord. 17(11-12):913-8, 2007.
Young et al., J Neuromuscul Dis. 4(2): 139-45, 2017.
Ran et al., Nat Protoc. 8(11):2281-308, 2013.
Burridge et al., Nat Methods. 11(8):855-60, 2014.

1. A composition comprising:
a sequence encoding a first DMD guide RNA (gRNA) targeting a first genomic target sequence;
a sequence encoding a second DMD gRNA targeting a second genomic target sequence;
a sequence encoding a first promoter, wherein the first promoter drives expression of the sequence encoding the first DMD gRNA; and
a sequence encoding a second promoter, wherein the second promoter drives expression of the sequence encoding the second DMD gRNA;
wherein the first genomic target sequence is in any one of introns 2-9 of the dystrophin gene;
wherein the second genomic target sequence is in any one of introns 2-9 of the dystrophin gene.

2. The composition of claim 1, wherein the first genomic target sequence is in intron 2 and the second genomic target sequence is in intron 7 of the dystrophin gene.

3. The composition of claim 1, wherein the first genomic target sequence is in intron 5 and the second genomic target sequence is in intron 7 of the dystrophin gene.

4. The composition of claim 1, wherein the first genomic target sequence is in intron 2 and the second genomic target sequence is in intron 9 of the dystrophin gene.

5. The composition of claim 1, wherein the first genomic target sequence is in intron 7 and the second genomic target sequence is in intron 9 of the dystrophin gene.

6. The composition of claim 1, wherein the first genomic target sequence is located 5′ from a wildtype exon, and the second genomic target sequence is located 3′ from the wildtype exon.

7. The composition of claim 6, wherein the wildtype exon is exon 2, 3, 4, 5, 6, 7, 8 or 9 of dystrophin.

8. The composition of claim 1, wherein the first genomic target sequence is located 5′ from an exon comprising a mutation, and the second genomic target sequence is located 3′ from the exon comprising a mutation.

9. The composition of claim 8, wherein the exon comprising a mutation is exon 2, 3, 4, 5, 6, 7, 8 or 9 of dystrophin.

10. The composition of claim 1, wherein the sequence encoding the first DMD gRNA and the sequence encoding the second DMD gRNA are identical.

11. The composition of claim 1, wherein the sequence encoding the first DMD gRNA and the sequence encoding the second DMD gRNA are not identical.

12. The composition of claim 1, wherein the sequence encoding the first DMD guide RNA is any one of SEQ ID NO: 1 to 5.

13. The composition of claim 1, wherein the sequence encoding the second DMD guide RNA is any one of SEQ ID NO: 1 to 5.

14. The composition of claim 1, wherein the sequence of the first DMD guide RNA is any one of SEQ ID NO: 6 to 10.

15. The composition of claim 1, wherein the sequence of the second DMD guide RNA is any one of SEQ ID NO: 6 to 10.

16. The composition of claim 1, wherein the sequence encoding the first promoter and the sequence encoding the second promoter are identical.

17. The composition of claim 1, wherein the sequence encoding the first promoter and the sequence encoding the second promoter are not identical.

18. The composition of claim 1, wherein at least one of the first promoter and the second promoter is a constitutive promoter.

19. The composition of claim 1, wherein at least one of the first promoter and the second promoter is an inducible promoter.

20. The composition of claim 1, wherein at least one of the first promoter and the second promoter is a muscle-specific promoter.

21. The composition of claim 20, wherein at least one of the first promoter and the second promoter is CK8.

22. The composition of claim 20, wherein at least one of the first promoter and the second promoter is CK8e.

23. The composition of claim 1, wherein at least one of the first promoter and the second promoter is selected from the group consisting of the U6 promoter, the H1 promoter, and the 7SK promoter.

24. The composition of claim 1, wherein a first vector comprises the sequence encoding the first DMD gRNA and the first promoter, and a second vector comprises the sequence encoding the second DMD gRNA and the second promoter.

25. The composition of claim 24, wherein the at least one of the first vector and the second vector is a non-viral vector.

26. The composition of claim 25, wherein the non-viral vector is a plasmid.

27. The composition of claim 25, wherein a liposome or nanoparticle comprises the non-viral vector.

28. The composition of claim 24, wherein at least one of the first vector and the second vector is a viral vector.

29. The composition of claim 28, wherein the viral vector is an adeno-associated viral (AAV) vector.

30. The composition of claim 29, wherein the AAV vector is replication-defective or conditionally replication defective.

31. The composition of claim 29, wherein the AAV vector is a recombinant AAV vector.

32. The composition of claim 29, wherein the AAV vector comprises a sequence isolated or derived from an AAV vector of serotype AAV1, AAV2, AAV3, AAV4, AAV5, AAV6, AAV7, AAV8, AAV9, AAV10, AAV11, AAVRh.74 or any combination thereof.

33. The composition of claim 1, wherein a first vector comprises the sequence encoding the first DMD gRNA, the sequence encoding the second DMD gRNA, the sequence encoding the first promoter, and the sequence encoding the second promoter.

34. The composition of claim 33, wherein the vector is a non-viral vector.

35. The composition of claim 34, wherein the non-viral vector is a plasmid.

36. The composition of claim 33, wherein the vector is a viral vector.

37. The composition of claim 36, wherein the viral vector is an adeno-associated viral (AAV) vector.

38. The composition of claim 37, wherein the AAV vector is replication-defective or conditionally replication defective.

39. The composition of claim 37, wherein the AAV vector is a recombinant AAV vector.

40. The composition of claim 37, wherein the AAV vector comprises a sequence isolated or derived from an AAV vector of serotype AAV1, AAV2, AAV3, AAV4, AAV5, AAV6, AAV7, AAV8, AAV9, AAV10, AAV11, AAVRh.74 or any combination thereof.

41. The composition of claim 1, wherein the composition further comprises a sequence encoding a nuclease.

42. The composition of claim 41, wherein the nuclease is Cas9.

43. The composition of claim 42, wherein the sequence encoding the Cas9 is isolated or derived from S. aureus.

44. The composition of claim 1, further comprising a pharmaceutically-acceptable carrier.

45. A cell comprising the composition of claim 1.

46. The cell of claim 45, wherein the cell is a mammalian cell.

47. The cell of claim 46, wherein the cell is a murine cell.

48. The cell of claim 46, wherein the cell is a human cell.

49. The cell of claim 45, wherein the cell is an oocyte.

50. A composition comprising the cell of claim 45.

51. A genetically engineered mouse comprising the cell of claim 45.

52. A method of treating a subject in need thereof, the method comprising administering to the subject a therapeutically effective amount of the composition of claim 1.

53. The method of claim 52, wherein the composition is administered locally.

54. The method of claim 52, wherein the composition is administered directly to a muscle tissue.

55. The method of claim 54, wherein the composition is administered by an intramuscular infusion or injection.

56. The method of claim 54, wherein the muscle tissue comprises a tibialis anterior tissue, a quadriceps tissue, a soleus tissue, a diaphragm tissue, or a heart tissue.

57. The method of claim 54, wherein the composition is administered by intra-cardiac injection.

58. The method of claim 52, wherein the composition is administered systemically.

59. The method of claim 58, wherein the composition is administered by an intravenous infusion or injection.

60. The method of claim 52, wherein the subject is a neonate, an infant, a child, a young adult, or an adult.

61. The method of claim 52, wherein the subject has muscular dystrophy.

62. The method of claim 52, wherein the subject is a genetic carrier for muscular dystrophy.

63. The method of claim 52, wherein the subject is male.

64. The method of claim 52, wherein the subject is female.

65. The method of claim 52, wherein the subject is less than 10 years old.

66. The method of claim 65, wherein the subject is less than 5 years old.

67. The method of claim 66, wherein the subject is less than 2 years old.

68. The use of the composition of claim 1 in the manufacture of a medicament for the treatment of muscular dystrophy.

69. The composition of claim 1 for use as a medicament in a method of treating muscular dystrophy.

70. A genetically engineered mouse whose genome comprises a deletion of exon 8 and 9 of the dystrophin gene resulting in an out of frame shift and a premature stop codon in exon 10.

71. A genetically engineered mouse produced by a method comprising the steps of:
(a) contacting a fertilized oocyte with (i) a Cas9, a first gRNA, and a second gRNA, or (ii) one or more sequences encoding the same, thereby creating a modified oocyte,
wherein the first gRNA targets an intron located 5′ to exon 8 of the dystrophin gene, wherein the second gRNA targets an intron located 3′ to exon 9 of the dystrophin gene,
wherein the contacting causes exons 8 and 9 to be deleted in the modified oocyte, wherein deletion of exons 8 and 9 results in an out of frame shift and a premature stop codon in exon 10;
(b) transferring the modified oocyte into a recipient female.

72. A mouse produced by the method of claim 71.

73. A method of editing an Actin Binding Domain 1 (ABD-1) dystrophin gene defect in a subject comprising contacting a cell with one or more expression constructs expressing Cas9, a first guide RNA and a second guide RNA, wherein the first guide RNA targets a dystrophin intron 5′ to the gene defect, and the second guide RNA targets a dystrophin intron 3′ to the gene defect, thereby resulting in an edited dystrophin gene lacking dystrophin exons 3-9.

74. The method of claim 73, wherein the cell is a muscle cell, a satellite cell, or an iPSC/iPSC-derived CM.

75. The method of claim 73, wherein the Cas9 expression construct is distinct from the expression construct that expresses the first and/or second guide RNAs.

76. The method of claim 73, wherein the Cas9 expression construct is the same expression construct as that expressing the first and/or second guide RNAs.

77. The method of claim 73, wherein the expression construct(s) is/are a viral vector.

78. The method of claim 73, wherein the expression construct(s) is/are a non-viral vector.

79. The method of claim 73, wherein the expression construct(s) is/are naked plasmid DNA or chemically-modified mRNA.

80. The method of claim 73, wherein (a) the first guide RNA targets dystrophin intron 2 and the second guide RNA targets dystrophin intron 9; (b) the first guide RNA targets dystrophin intron 2, and the second guide RNA targets dystrophin intron 7; (c) the first guide RNA targets dystrophin intron 7, and the second guide RNA targets dystrophin intron 9.

81. The method of claim 73, wherein the expression construct(s) is/are provided to the cell in one or more nanoparticles.

82. The method of claim 77, wherein the viral vector is an AAV vector, such as AAV-9.

83. The method of claim 79, wherein contacting comprises administration of AAV vector to the subject, such as by intra-muscular, intra-peritoneal (IP), retro-orbital (RO), or intra-cardiac injection.

84. The method of claim 73, wherein the expression construct(s) is/are delivered to and iPSC/iPSC-derived CM or directly to a muscle tissue.

85. The method of claim 84, wherein the muscle tissue is tibialis anterior, quadriceps, soleus, diaphragm or heart.

86. The method of claim 73, wherein the expression construct(s) is/are delivered systemically.

87. The method of claim 73, wherein the subject exhibits normal dystrophin-positive myofibers and/or mosaic dystrophin-positive myofibers containing centralized nuclei.

88. The method of claim 73, wherein the subject exhibits a decreased serum CK level as compared to a serum CK level prior to contacting.

89. The method of claim 73, wherein the subject exhibits improved grip strength as compared to a serum CK level prior to contacting.

90. The method of claim 73, wherein the first guide RNA is encoded by the DNA sequence 5′-AATTAATCTGCCGAAGATGA-3′ (SEQ ID NO: 1).

91. The method of claim 90, wherein the second guide RNA is encoded by the DNA sequence 5′-AAACAAACCAGCTCTTCACG-3′ (SEQ ID NO: 5).

92. The method of claim 90, wherein the expression construct(s) is/are delivered to a human iPS cell by nucleofection.

93. The method claim 73, further comprising identifying an ABD-1 target based on reference to a Duchenne mutation database.

94. The method of claim 73, wherein the first and second guide RNAs are encoded by and expressed from the same expression construct.

95. The method of claim 73, wherein the first and second guide RNAs are encoded by and expressed from distinct expression constructs.

96. The method of claim 73, wherein one or more promoters in the expression construct(s) is/are RNA polymerase III promoters.

97. The method of claim 73, wherein the mutant dystrophin exon is exon 3, 4, 5, 6, 7, 8 or 9.
